SEQUENCE LISTING (6 sequences)

SEQ ID NO: 1 - protein, 169 aa, Homo sapiens
MAPPWVPAMGFTLAPSLGCFVGSRFVHGEGLRWYAGLQKPSWHPPHWVLGPVWGTLYSAMGYGSYLVWKELGGFTEKAVVPLGLYTGQLALNWAWPPIFFGARQMGWALVDLLLVSGAAAATTVAWYQVSPLAARLLYPYLAWLAFTTTLNYCVWRDNHGWRGGRRLPE

SEQ ID NO: 2 - DNA, 921 bp, Homo sapiens
  source 1..921: mol_type = genomic DNA
ggcggctgggaggggcggggcggatgcggggacagcggcctggctaactcctgccaggcagtgcccttcccggagcgtgccctcgccgctgagctcccctgaacagcagctgcagcagccatggccccgccctgggtgcccgccatgggcttcacgctggcgcccagcctggggtgcttcgtgggctcccgctttgtccacggcgagggtctccgctggtacgccggcctgcagaagccctcgtggcacccgccccactgggtgctgggccctgtctggggcacgctctactcagccatggggtacggctcctacctggtctggaaagagctgggaggcttcacagagaaggctgtggttcccctgggcctctacactgggcagctggccctgaactgggcatggccccccatcttctttggtgcccgacaaatgggctgggccttggtggatctcctgctggtcagtggggcggcggcagccactaccgtggcctggtaccaggtgagcccgctggccgcccgcctgctctacccctacctggcctggctggccttcacgaccacactcaactactgcgtatggcgggacaaccatggctggcgtgggggacggcggctgccagagtgagtgcccggcccaccagggactgcagctgcaccagcaggtgccatcacgcttgtgatgtggtggccgtcacgctttcatgaccactgggcctgctagtctgtcagggccttggcccaggggtcagcagagcttcagaggtggccccacctgagcccccacccgggagcagtgtcctgtgctttctgcatgcttagagcatgttcttggaacatggaattttataagctgaataaagtttttgacttcctttaaaaaaaaaaaaaaaaaaaaaaaaaaaaaaaaaaaaaaaaa

SEQ ID NO: 3 - protein, 595 aa, Homo sapiens
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVYEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 4 - DNA, 3680 bp, Homo sapiens
  source 1..3680: mol_type = genomic DNA
gtcattggaggagcttgaagttaaagactcctgctaaaaaccagtacgtttcattttgcagttactgggagggggcttgctgtggccctgtcaggaagagtagagctctggtccagctccgcgcagggagggaggctgtcaccatgccggcctgctgcagctgcagtgatgttttccagtatgagacgaacaaagtcactcggatccagagcatgaattatggcaccattaagtggttcttccacgtgatcatcttttcctacgtttgctttgctctggtgagtgacaagctgtaccagcggaaagagcctgtcatcagttctgtgcacaccaaggtgaaggggatagcagaggtgaaagaggagatcgtggagaatggagtgaagaagttggtgcacagtgtctttgacaccgcagactacaccttccctttgcaggggaactctttcttcgtgatgacaaactttctcaaaacagaaggccaagagcagcggttgtgtcccgagtatcccacccgcaggacgctctgttcctctgaccgaggttgtaaaaagggatggatggacccgcagagcaaaggaattcagaccggaaggtgtgtagtgtatgaagggaaccagaagacctgtgaagtctctgcctggtgccccatcgaggcagtggaagaggccccccggcctgctctcttgaacagtgccgaaaacttcactgtgctcatcaagaacaatatcgacttccccggccacaactacaccacgagaaacatcctgccaggtttaaacatcacttgtaccttccacaagactcagaatccacagtgtcccattttccgactaggagacatcttccgagaaacaggcgataatttttcagatgtggcaattcagggcggaataatgggcattgagatctactgggactgcaacctagaccgttggttccatcactgccgtcccaaatacagtttccgtcgccttgacgacaagaccaccaacgtgtccttgtaccctggctacaacttcagatacgccaagtactacaaggaaaacaatgttgagaaacggactctgataaaagtcttcgggatccgttttgacatcctggtttttggcaccggaggaaaatttgacattatccagctggttgtgtacatcggctcaaccctctcctacttcggtctggccgctgtgttcatcgacttcctcatcgacacttactccagtaactgctgtcgctcccatatttatccctggtgcaagtgctgtcagccctgtgtggtcaacgaatactactacaggaagaagtgcgagtccattgtggagccaaagccgacattaaagtatgtgtcctttgtggatgaatcccacattaggatggtgaaccagcagctactagggagaagtctgcaagatgtcaagggccaagaagtcccaagacctgcgatggacttcacagatttgtccaggctgcccctggccctccatgacacacccccgattcctggacaaccagaggagatacagctgcttagaaaggaggcgactcctagatccagggatagccccgtctggtgccagtgtggaagctgcctcccatctcaactccctgagagccacaggtgcctggaggagctgtgctgccggaaaaagccgggggcctgcatcaccacctcagagctgttcaggaagctggtcctgtccagacacgtcctgcagttcctcctgctctaccaggagcccttgctggcgctggatgtggattccaccaacagccggctgcggcactgtgcctacaggtgctacgccacctggcgcttcggctcccaggacatggctgactttgccatcctgcccagctgctgccgctggaggatccggaaagagtttccgaagagtgaagggcagtacagtggcttcaagagtccttactgaagccaggcaccgtggctcacgtctgtaatcccagcgctttgggaggccgaggcaggcagatcacctgaggtcgggagttggagacccgcctggctaacaaggcgaaatcctgtctgtactaaaaatacaaaaatcagccagacatggtggcatgcacctgcaatcccagctactcgggaggctgaggcacaagaatcacttgaacccgggaggcagaggttgtagtgagcccagattgtgccactgctctccagcctgggaggcacagcaaactgtcccccaaaaaaaaaaaagagtccttaccaatagcaggggctgcagtagccatgttaacatgacatttaccagcaacttgaacttcacctgcaaagctctgtggccacattttcagccaaagggaaatatgctttcatcttctgttgctctctgtgtctgagagcaaagtgacctggttaaacaaaccagaatccctctacatggactcagagaaaagagattgagatgtaagtctcaactctgtccccaggaagttgtgtgaccctaggcctctcacctctgtgcctctgtctccttgttgcccaactactatctcagagatattgtgaggacaaattgagacagtgcacatgaactgtcttttaatgtgtaaagatctacatgaatgcaaaacatttcattatgaggtcagactaggataatgtccaactaaaaacaaacccttttcatcctggctggagaatgtggagaactaaaggtggccacaaattctttgacactcaagtcccccaagacctaagggttttatctcctccccttgaatatgggtggctctgattgctttatccaaaagtggaagtgacattgtgtcagtttcagatcctgatcttaagaggctgacagcttctacttgctgtcccttggaactcttgctatcggggaagccagacgccatttaaaagtctgcctatcctggccaggtgtggtggctcacacctgtaatcccagcactttgggagaccaaggcgggcggatcacttaaagtcaggagtccaagaccagactcgccaacatggtgaaaccgtatctctaataaaaatacaaaaattagctgggcatggtgcgggcacctgtagtcctagctatcaagaggctgagacaggagaaacacttgaacctgggaggtggaggttgcattgagctgagatcgtgccactgcactccaggctgggtgacagagcgagactccatctcaaaaaaaaaaaaaagaaaaaaaaaatgtctgcctatcctgagactgccctgctgtgaggaagcccaagcagtcacgtggacagtgcctgaccagccccagctttcaagccatccaagcccagtcaccaaacatgagagagaagaagccttcaggtgattctggactccactaacatatgactgataccgcatgatacatcccaagtgagaactgccccataaatccagaaaaccacattgctatcttaagtccctaagtttggggcttatttgttccacagcaacaggtaactggaacagagggcaagcctgatgaatgggcacacagactcagcccataccttccctggttctaatgttctcagggagcccggaccaaccctgggagcctcaggaacttaggtttccactggacagttctagaagggctatagaccaaatcaggtaactcaccagaccagccttggaatctatcaaatctaactgctgagctaccca

SEQ ID NO: 5 - protein, 360 aa, Homo sapiens
MEECWVTEIANGSKDGLDSNPMKDYMILSGPQKTAVAVLCTLLGLLSALENVAVLYLILSSHQLRRKPSYLFIGSLAGADFLASVVFACSFVNFHVFHGVDSKAVFLLKIGSVTMTFTASVGSLLLTAIDRYLCLRYPPSYKALLTRGRALVTLGIMWVLSALVSYLPLMGWTCCPRPCSELFPLIPNDYLLSWLLFIAFLFSGIIYTYGHVLWKAHQHVASLSGHQDRQVPGMARMRLDVRLAKTLGLVLAVLLICWFPVLALMAHSLATTLSDQVKKAFAFCSMLCLINSMVNPVIYALRSGEIRSSAHHCLAHWKKCVRGLGSEAKEEAPRSSVTETEADGKITPWPDSRDLDLSDC

SEQ ID NO: 6 - DNA, 1789 bp, Homo sapiens
  source 1..1789: mol_type = genomic DNA
caggtcctgggagaggacagaaaacaactgggactcctcagcccccggcagctcccagtgcccagccacccacaacacaacccaaagccttctagacaagctcagtggaatctgaagggcccaccccatggaggaatgctgggtgacagagatagccaatggctccaaggatggcttggattccaaccctatgaaggattacatgatcctgagtggtccccagaagacagctgttgctgtgttgtgcactcttctgggcctgctaagtgccctggagaacgtggctgtgctctatctgatcctgtcctcccaccaactccgccggaagccctcatacctgttcattggcagcttggctggggctgacttcctggccagtgtggtctttgcatgcagctttgtgaatttccatgttttccatggtgtggattccaaggctgtcttcctgctgaagattggcagcgtgactatgaccttcacagcctctgtgggtagcctcctgctgaccgccattgaccgatacctctgcctgcgctatccaccttcctacaaagctctgctcacccgtggaagggcactggtgaccctgggcatcatgtgggtcctctcagcactagtctcctacctgcccctcatgggatggacttgctgtcccaggccctgctctgagcttttcccactgatccccaatgactacctgctgagctggctcctgttcatcgccttcctcttttccggaatcatctacacctatgggcatgttctctggaaggcccatcagcatgtggccagcttgtctggccaccaggacaggcaggtgccaggaatggcccgaatgaggctggatgtgaggttggccaagaccctagggctagtgttggctgtgctcctcatctgttggttcccagtgctggccctcatggcccacagcctggccactacgctcagtgaccaggtcaagaaggcctttgctttctgctccatgctgtgcctcatcaactccatggtcaaccctgtcatctatgctctacggagtggagagatccgctcctctgcccatcactgcctggctcactggaagaagtgtgtgaggggccttgggtcagaggcaaaagaagaagccccgagatcctcagtcaccgagacagaggctgatgggaaaatcactccgtggccagattccagagatctagacctctctgattgctgatgaggcctcttcccaatttaaacaactcaagtcagaaatcagttcactccctggaagagagagaggggtcttggcactctcttcttacttaaaccagtcccagacacctagacacggacccctttttgctgatgagtgttgggactgactcctggaagacagcctggccttgcccacctgcacacagtctgttggataggtagggccacgaggagtagccaggtaggcgagacacaaaaggcctgggacagggtcagtacaagtcaggtcaggcttcatgcctgcatcctccagagaccacaggagccaaagcgagcctccaggcccagcaatgagggacttgggagaaatctgagaagaatgggttgttctcttgggaagtcagggtatcagatgggatggacatccaggtcttctctctgcctaattgtcaaggcctccttggctctggagctatgaaaggccccactttcaagtcacccttgccactgaggaccgaggactatgctatgatgaggattaaggtgttgacttgcctctttcagagataaatgacaagccttcaaaaaaaaaaaaaaa

(19) United States
(12) Patent Application Publication
(10) Pub. No.: US 2025/0049839 A1
(43) Pub. Date: Feb. 13, 2025

(54) COMPOSITIONS AND METHODS FOR TARGETING CELLS

(71) Applicants: Children's Medical Center Corporation, Boston, MA (US); Politecnico di Milano, Milano (IT); Dana-Farber Cancer Institute, Inc., Boston, MA (US)

(72) Inventors: Marco Peviani, Boston, MA (US); Alessandra Biffi, Boston, MA (US); Davide Moscatelli, Milano (IT); Umberto Capasso Palmiero, Milano (IT); Renato Auriemma, Milano (IT); Mattia Sponchioni, Milano (IT); Letterio Salvatore Politi, Milano (IT)

(73) Assignees: Children's Medical Center Corporation, Boston, MA (US); Politecnico di Milano, Milano (IT); Dana-Farber Cancer Institute, Inc., Boston, MA (US)

(21) Appl. No.: 18/933,959

(22) Filed: Oct. 31, 2024

Related U.S. Application Data
(62) Division of application No. 17/035,339, filed on Sep. 28, 2020, now Pat. No. 12,161,663.
Application No. 17/035,339 is a continuation of application No. PCT/US2019/024944, filed on Mar. 29, 2019.
(60) Provisional application No. 62/650,207, filed on Mar. 29, 2018.

(51) Int. Cl. A61K 31/785; A61K 9/14; A61K 31/655; A61K 31/662; A61K 47/54; A61K 47/58; A61K 47/69; A61K 49/08; A61K 49/12; A61K 49/18; A61K 51/06; A61K 51/12; C08F 290/06

(52) U.S. Cl. CPC A61K 31/785; A61K 9/145; A61K 31/655; A61K 31/662; A61K 47/545; A61K 47/58; A61K 47/6933; A61K 47/6935; A61K 49/085; A61K 49/128; A61K 49/1824; A61K 49/1854; A61K 51/065; A61K 51/1244; C08F 290/061

(57) ABSTRACT

The present invention provides compositions and methods for targeting cells for therapeutic and/or diagnostic purposes, e.g., delivery of therapeutic and/or diagnostic agents to a cell. Nanoparticles and polymers functionalized with capture molecules, reporter molecules, and/or therapeutic agents are provided for the treatment or prevention of disease, including neurological diseases associated with neuroinflammation, and cancer.

### CROSS-REFERENCE TO RELATED APPLICATION

This application is a U.S. National Stage filed under 35 U.S.C. § 111(a), which is a continuation of and claims priority to PCT/US2019/024944, filed Mar. 29, 2019, which claims the benefit of and priority to U.S. Provisional Application No. 62/650,207, filed Mar. 29, 2018, the entire contents of each of which are incorporated herein by reference.

### STATEMENT OF RIGHTS TO INVENTIONS MADE UNDER FEDERALLY SPONSORED RESEARCH

This invention was made with government support under Grant No. W81XWH-17-1-0036 awarded by the Department of Defense. The government has certain rights in the invention.

### BACKGROUND OF THE INVENTION

Accumulating lines of evidence suggest that neuroinflammation, i.e., the activation of glial cells in the central nervous system (CNS), is not just the result of reaction to neuronal death or damage, but is a key pathological process that actively contributes to the worsening and progression of symptoms in several neurodegenerative diseases, such as acute brain or spinal cord injury, and Amyotrophic Lateral Sclerosis (ALS).

The current therapeutic approaches for ALS are based on chronic administration of neuroprotective factors (e.g., trophic or anti-apoptotic factors, treatment with anti-glutamatergic drugs, or compounds enhancing proteasome or mitochondrial metabolic activity) or anti-inflammatory molecules (e.g., cyclooxygenase inhibitors or minocycline) among others. These strategies suffer from limited efficacy, possibly due to induction of pharmacological tolerance or to the lack of specificity for particular cell types (e.g., glia versus neurons or neurotoxic versus neuro-supportive microglia). Moreover, these strategies do not take into account the complexity of ALS pathology, such as compensatory neuro-supportive responses that co-exist with, or eventually evolve to, pro-degenerative cellular and molecular responses in different CNS districts. In fact, the most common initial presentation of ALS is focal asymmetric distal weakness accompanied by muscular atrophy, which reflects the presence of specific foci of neuronal dysfunction in restricted CNS areas. The organism senses this damage and initially tries to compensate for the neuronal loss, for example, by compensatory reinnervation from nearby motor neurons, which permits maintenance of motor function until more than 50% of motor units have been lost. However, over time this scenario eventually worsens. Damage spreading to other neuronal districts causes progressive deterioration of motor function, which eventually leads to a fatal outcome.

Microglia cells, the innate immune cells in the CNS, have recently gained great interest as a potential target for several therapeutic approaches to neurodegenerative disease. For example, fine tuning microglia/macrophage reactivity may play a key role in modulating neuroinflammatory processes, thereby producing potential significant therapeutic benefits. The major challenge for developing such therapeutic approaches is to achieve selective targeting of reactive microglia/macrophages in the CNS. Accordingly, new compositions and methods of treatment for neurodegenerative disease are urgently required.

### SUMMARY OF THE INVENTION

As described below, the present invention features compositions and methods for targeting and delivery of therapeutic and/or diagnostic agents to a cell (e.g., microglia, tumor cell). The invention generally provides the use of nanoparticles and polymers functionalized with capture molecules, reporter molecules, and/or therapeutic agents for the treatment or prevention of disease (e.g., cancer, neurological diseases associated with neuroinflammation).

In one aspect, the invention provides a polymer or functionalized polymer having a first monomer: 2-Methacryloyloxyethyl phosphorylcholine (MPC), and a second monomer: methacrylate/amide PEG including a functionalized amine or azide, where one or more of a capture reagent and detectable is covalently linked via the functionalized amine or azide.

In another aspect the invention provides a method of making a polymer or functionalized polymer involving contacting a first monomer: 2-Methacryloyloxyethyl phosphorylcholine (MPC), and a second monomer: methacrylate/amide PEG having a functionalized amine or azide, in the presence of a radical and a thiocarbonylthio compound.

In another aspect, the invention provides a polymer or functionalized polymer made according to the method of any aspect of the invention.

In various embodiments of any aspect delineated herein, the polymer comprises Formula I

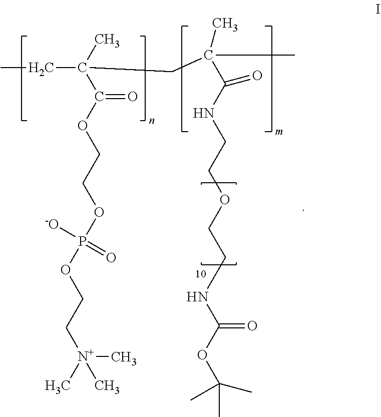

In various embodiments of any aspect delineated herein, the ratio of the first monomer to the second monomer is about 25:3 to about 25:6. In various embodiments of any aspect delineated herein, the functionalized amine is protected by a tert-Butyloxycarbonyl group.

In various embodiments of any aspect delineated herein, the polymer includes a third monomer: hydroxyethyl methacrylate polycaprolactone (HEMA-PCL) having a functionalized carboxyl group. In various embodiments, the polymer comprises Formula II:

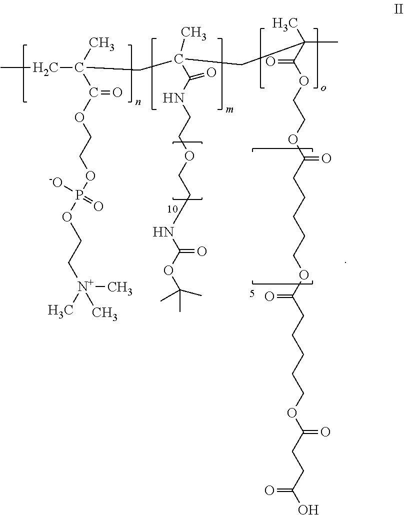

In various embodiments, the polymer contains a hydroxyethyl methacrylate-rhodamine (HEMA-Rhodamine) monomer. In various embodiments, the polymer contains a hydroxyethyl methacrylate-succinate (HEMA-succinate) monomer.

In another aspect, the invention provides a nanoparticle containing a polymer according to any aspect of the invention.

In another aspect, the invention provides a method of targeting a cell involving contacting the cell with a nanoparticle according to any aspect of the invention.

In another aspect, the invention provides a polymer or functionalized polymer containing a TSPO ligand or precursor or analog thereof covalently linked to a branched PEG polymer. In various embodiments, the TSPO ligand is one or more of PK11195, PBR28, Ro5-4864, GE180, FGIN-1-27, Alpidem, DPA-714, or precursor or analog thereof. In various embodiments, polymer or functionalized polymer includes a detectable moiety covalently linked to the branched PEG polymer. In various embodiments, the detectable moiety is a fluorescent dye, rhodamine, Fluorescein isothiocyanate (FITC), Cy5, or aminomethylcoumarin acetate (AMCA).

In another aspect, the invention provides a nanoparticle containing a polymer and iron, where the polymer contains a first monomer: 2-Methacryloyloxyethyl phosphorylcholine (MPC), and a second monomer: methacrylate PEG having a covalently linked diapocynin.

In another aspect, the invention provides a nanoparticle containing a polymer, where the polymer contains a first monomer: 2-Methacryloyloxyethyl phosphorylcholine (MPC), a second monomer: methacrylate PEG having a covalently linked diapocynin, and a third monomer: hydroxyethyl methacrylate-deferoxamine (HEMA-deferoxamine), where the deferoxamine is bound to Zirconium⁸⁹.

In another aspect, the invention provides a nanoparticle containing a biodegradable polycation or ionizable polymer containing a poly(O-amino ester) (PBAE), and a nucleic acid molecule, wherein the polymer comprises a first monomer: 2-Methacryloyloxyethyl phosphorylcholine (MPC), and a second monomer: methacrylate PEG.

In another aspect, the invention provides a method of detecting a cell involving contacting the cell with a nanoparticle containing a polymer, where the polymer contains a first monomer: 2-Methacryloyloxyethyl phosphorylcholine (MPC); a second monomer: methacrylate PEG comprising a covalently linked TSPO ligand, PK11195, PBR28, or a precursor or analog thereof, and a third monomer: hydroxyethyl methacrylate-deferoxamine (HEMA-deferoxamine), wherein the deferoxamine is bound to Zirconium⁸⁹.

In another aspect, the invention provides a method of detecting a cell involving contacting the cell with a nanoparticle containing a polymer and iron, where the polymer contains a first monomer: 2-Methacryloyloxyethyl phosphorylcholine (MPC), and a second monomer: methacrylate PEG comprising a covalently linked TSPO ligand, PK11195, PBR28, or a precursor or analog thereof.

In another aspect, the invention provides a method of detecting a cell involving contacting the cell with a nanoparticle containing a polymer, Poly(O-amino ester), and a nucleic acid molecule, where the polymer contains a first monomer: 2-Methacryloyloxyethyl phosphorylcholine (MPC), and a second monomer: methacrylate PEG; where the nucleic acid molecule expresses a fluorescent protein or green fluorescent protein (GFP).

In another aspect, the invention provides a method of delivering an agent to a cell involving contacting the cell with nanoparticle containing a polymer and the agent, where the polymer contains a first monomer: 2-Methacryloyloxyethyl phosphorylcholine (MPC), and a second monomer: methacrylate PEG covalently linked to one or more of a capture reagent, detectable moiety, or combination thereof.

In another aspect, the invention provides a method of delivering a nucleic acid molecule to a cell involving contacting the cell with a nanoparticle containing a polymer, Poly(O-amino ester), and a nucleic acid molecule, where the polymer contains a first monomer: 2-Methacryloyloxyethyl phosphorylcholine (MPC), and a second monomer: methacrylate PEG covalently linked to one or more of a capture reagent, detectable moiety, or combination thereof.

In another aspect, the invention provides a method of treating neuroinflammation in a subject involving administering to the subject a nanoparticle containing a polymer, where the polymer contains a first monomer: 2-Methacryloyloxyethyl phosphorylcholine (MPC), and a second monomer: methacrylate PEG having a covalently linked TSPO ligand, PK11195, PBR28, or a precursor or analog thereof, and a third monomer: hydroxyethyl methacrylate-deferoxamine (HEMA-deferoxamine), where the deferoxamine is bound to Zirconium⁸9.

In another aspect, the invention provides a method of treating neuroinflammation in a subject involving administering to the subject a nanoparticle containing a polymer and iron, where the polymer contains a first monomer: 2-Methacryloyloxyethyl phosphorylcholine (MPC), and a second monomer: methacrylate PEG having a covalently linked TSPO ligand, PK11195, PBR28, or a precursor or analog thereof.

In another aspect, the invention provides a method of treating cancer in a subject involving administering to the subject a nanoparticle containing a polymer and a chemotherapeutic agent, where the polymer contains a first monomer: 2-Methacryloyloxyethyl phosphorylcholine (MPC), and a second monomer: methacrylate PEG having a covalently linked TSPO ligand, PK11195, PBR28, or a precursor or analog thereof, and wherein the chemotherapeutic agent is selected from etoposide, busulfan, and lomustine.

In various embodiments of any aspect delineated herein, the nanoparticle contains iron (e.g., an Fe ion). In various embodiments of any aspect delineated herein, the nanoparticle contains hydroxyethyl methacrylate covalently linked to a chelator; deferoxamine (DFO); 1,4,7,10-Tetraazacyclododecane-1,4,7,10-tetraacetic acid; Tetraxetan; or EDTA. In various embodiments, the deferoxamine (DFO) is bound to a radioisotope, including e.g., Zirconium⁸⁹, Fluoride¹⁸, or Carbonium¹¹. In various embodiments of any aspect delineated herein, the nanoparticle contains biodegradable polycation or ionizable polymer, comprising a poly(β-amino ester) (PBAE) polymer, wherein the PBAE polymer comprises polycaprolactone (PCL).

In various embodiments of any aspect delineated herein, the nanoparticle contains a nucleic acid molecule, polypeptide, or small molecule. In various embodiments, the nucleic acid molecule is a plasmid, vector, inhibitory nucleic acid, antisense oligonucleotide, or small interfering RNA (siRNA). In various embodiments, the nucleic acid molecule expresses a polypeptide or polynucleotide, including a therapeutic or reporter polypeptide, e.g., a fluorescent protein, green fluorescent protein (GFP), metallothionein, or Insulin growth factor (IGF1). In various embodiments of any aspect delineated herein, the nucleic acid molecule, polypeptide, or small molecule is directly conjugated to the nanoparticle. In various embodiments of any aspect delineated herein, the nucleic acid molecule, polypeptide, or small molecule is loaded in the nanoparticle.

In various embodiments of any aspect delineated herein, the nanoparticle contains a capture molecule or binding agent. In various embodiments, a cell (e.g., microglia, cancer cell, etc.) is targeted. In various embodiments, the targeting occurs in vitro or in vivo. In particular embodiments, the nanoparticle is administered to a subject (e.g., by Intra-cerebral Ventricular Injection (ICV) or intrathecal administration (ITL). In certain embodiments, the nanoparticle contains a Translocator protein (TSPO) ligand or capture molecule, including but not limited to, PK11195, PBR28, Ro5-4864, GE180, FGIN-1-27, Alpidem, DPA-714, or a precursor or analog thereof. In various embodiments of any aspect delineated herein, the nanoparticle targets a cell. In certain embodiments, a cell surface protein or moiety is contacted with a capture molecule or ligand on the nanoparticle. In various embodiments, the cell surface protein or moiety is Translocator protein (TSPO), P2X purinoceptor 7 (P2X7r), Cannabinoid receptor type 2 (CB2r), CD68, fractalkine receptor CX3CR1, Glutamate aspartate transporter, proteoglycan NG2, oligodendrocyte antigen 04, CD31, CD90, or Acetylcholine receptor, or fragment thereof.

In various embodiments of any aspect delineated herein, a therapeutic agent or reporter is delivered (e.g., to a cell) using the nanoparticle. In various embodiments, the delivering occurs in vitro or in vivo. In certain embodiments, cell uptake of a therapeutic agent or reporter is provided using the nanoparticle. In certain embodiments, the therapeutic agent is an anti-inflammatory agent, e.g., diapocynin, or chemotherapeutic agent, e.g., etoposide, busulfan, or lomustine. In particular embodiments, the nanoparticle or component thereof is detectable by Positron Emission Tomography (PET) or Magnetic Resonance Imaging (MRI) (FIG. 1). In various embodiments, the detecting occurs in vitro or in vivo.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Translocator protein (TSPO) polypeptide” is meant a polypeptide or fragment thereof having at least about 85%, or greater, amino acid identity to NCBI Accession No. NP_000705 (below) and having binding activity to benzodiazepine, and analogs thereof.

| | |
|---|---|
| 1 | mappwvpamg ftlapslgcf vgsrfvhgeg lrwyaglqkp swhpphwvlg pvwgtlysam |
| 61 | gygsylvwke lggftekavv plglytggla lnwawppiff garqmgwalv dlllvsgaaa |
| 121 | attvawyqvs plaarllypy lawlaftttl nycvwrdnhg wrggrrlpe |

By “TSPO nucleic acid molecule” is meant a polynucleotide encoding a TSPO polypeptide. An exemplary TSPO nucleic acid molecule sequence is provided at NCBI Accession No. NM_000714 (below):

| | |
|---|---|
| 1 | ggcggctggg aggggcgggg cggatgcggg gacagcggcc tggctaactc ctgccaggca |
| 61 | gtgcccttcc cggagcgtgc cctcgccgct gagctcccct gaacagcagc tgcagcagcc |
| 121 | atggccccgc cctgggtgcc cgccatgggc ttcacgctgg cgcccagcct ggggtgcttc |
| 181 | gtgggctccc gctttgtcca cggcgagggt ctccgctggt acgccggcct gcagaagccc |
| 241 | tcgtggcacc cgccccactg ggtgctgggc cctgtctggg gcacgctcta ctcagccatg |
| 301 | gggtacggct cctacctggt ctggaaagag ctgggaggct tcacagagaa ggctgtggtt |
| 361 | cccctgggcc tctacactgg gcagctggcc ctgaactggg catggccccc catcttcttt |
| 421 | ggtgcccgac aaatgggctg ggccttggtg gatctcctgc tggtcagtgg ggcggcggca |
| 481 | gccactaccg tggcctggta ccaggtgagc ccgctggccg cccgcctgct ctacccctac |
| 541 | ctggcctggc tggccttcac gaccacactc aactactgcg tatggcggga caaccatggc |
| 601 | tggcgtgggg gacggcggct gccagagtga gtgcccggcc caccagggac tgcagctgca |
| 661 | ccagcaggtg ccatcacgct tgtgatgtgg tggccgtcac gctttcatga ccactgggcc |
| 721 | tgctagtctg tcagggcctt ggcccagggg tcagcagagc ttcagaggtg gccccacctg |
| 781 | agcccccacc cgggagcagt gtcctgtgct ttctgcatgc ttagagcatg ttcttggaac |
| 841 | atggaatttt ataagctgaa taaagttttt gacttccttt aaaaaaaaaa aaaaaaaaaa |
| 901 | aaaaaaaaaa aaaaaaaaaa a |

By “TSPO ligand” is meant an agent that specifically binds TSPO. Exemplary TSPO ligands are known in the art and include, but are not limited to, PK11195, PBR28, Ro5-4864, 15 GE180, FGIN-1-27, Alpidem, DPA-714, and analogs thereof.

By “P2X purinoceptor 7 (P2X7r) polypeptide” is meant a polypeptide or fragment thereof having at least about 85%, or greater, amino acid identity to NCBI Accession No. NP_002553 (below) and having binding activity to ATP, and analogs thereof.

| | |
|---|---|
| 1 | mpaccscsdv fqyetnkvtr iqsmnygtik wffhviifsy vcfalvsdkl yqrkepviss |
| 61 | vhtkvkgiae vkeeivengv kklvhsvfdt adytfplqgn sffvmtnflk tegqeqrlcp |
| 121 | eyptrrtlcs sdrgckkgwm dpqskgiqtg rcvvyegnqk tcevsawcpi eaveeaprpa |
| 181 | llnsaenftv liknnidfpg hnyttrnilp glnitctfhk tqnpqcpifr lgdifretgd |
| 241 | nfsdvaiqgg imgieiywdc nldrwfhhcr pkysfrrldd kttnvslypg ynfryakyyk |
| 301 | ennvekrtli kvfgirfdil vfgtggkfdi iqlvvyigst lsyfglaavf idflidtyss |
| 361 | nccrshiypw ckccqpcvvn eyyyrkkces ivepkptlky vsfvdeshir mvnqqllgrs |
| 421 | lqdvkgqevp rpamdftdls rlplalhdtp pipgqpeeiq llrkeatprs rdspvwcqcg |
| 481 | sclpsqlpes hrcleelccr kkpgacitts elfrklvlsr hvlqflllyq epllaldvds |
| 541 | tnsrlrhcay rcyatwrfgs qdmadfailp sccrwrirke fpksegqysg fkspy |

By “P2X7r nucleic acid molecule” is meant a polynucleotide encoding a P2X7r polypeptide. An exemplary P2X7r nucleic acid molecule sequence is provided at NCBI Accession No. NM_002562 (below):

| | |
|---|---|
| 1 | gtcattggag gagcttgaag ttaaagactc ctgctaaaaa ccagtacgtt tcattttgca |
| 61 | gttactggga gggggcttgc tgtggccctg tcaggaagag tagagctctg gtccagctcc |
| 121 | gcgcagggag ggaggctgtc accatgccgg cctgctgcag ctgcagtgat gttttccagt |
| 181 | atgagacgaa caaagtcact cggatccaga gcatgaatta tggcaccatt aagtggttct |
| 241 | tccacgtgat catcttttcc tacgtttgct ttgctctggt gagtgacaag ctgtaccagc |
| 301 | ggaaagagcc tgtcatcagt tctgtgcaca ccaaggtgaa ggggatagca gaggtgaaag |
| 361 | aggagatcgt ggagaatgga gtgaagaagt tggtgcacag tgtctttgac accgcagact |
| 421 | acaccttccc tttgcagggg aactctttct tcgtgatgac aaactttctc aaaacagaag |
| 481 | gccaagagca gcggttgtgt cccgagtatc ccacccgcag gacgctctgt tcctctgacc |
| 541 | gaggttgtaa aaagggatgg atggacccgc agagcaaagg aattcagacc ggaaggtgtg |
| 601 | tagtgtatga agggaaccag aagacctgtg aagtctctgc ctggtgcccc atcgaggcag |
| 661 | tggaagaggc cccccggcct gctctcttga acagtgccga aaacttcact gtgctcatca |
| 721 | agaacaatat cgacttcccc ggccacaact acaccacgag aaacatcctg ccaggtttaa |
| 781 | acatcacttg taccttccac aagactcaga atccacagtg tcccattttc cgactaggag |
| 841 | acatcttccg agaaacaggc gataattttt cagatgtggc aattcagggc ggaataatgg |
| 901 | gcattgagat ctactgggac tgcaacctag accgttggtt ccatcactgc cgtcccaaat |
| 961 | acagtttccg tcgccttgac gacaagacca ccaacgtgtc cttgtaccct ggctacaact |
| 1021 | tcagatacgc caagtactac aaggaaaaca atgttgagaa acggactctg ataaaagtct |
| 1081 | tcgggatccg ttttgacatc ctggtttttg gcaccggagg aaaatttgac attatccagc |
| 1141 | tggttgtgta catcggctca accctctcct acttcggtct ggccgctgtg ttcatcgact |
| 1201 | tcctcatcga cacttactcc agtaactgct gtcgctccca tatttatccc tggtgcaagt |
| 1261 | gctgtcagcc ctgtgtggtc aacgaatact actacaggaa gaagtgcgag tccattgtgg |
| 1321 | agccaaagcc gacattaaag tatgtgtcct ttgtggatga atcccacatt aggatggtga |
| 1381 | accagcagct actagggaga agtctgcaag atgtcaaggg ccaagaagtc ccaagacctg |
| 1441 | cgatggactt cacagatttg tccaggctgc ccctggccct ccatgacaca cccccgattc |
| 1501 | ctggacaacc agaggagata cagctgctta gaaaggaggc gactcctaga tccagggata |
| 1561 | gccccgtctg gtgccagtgt ggaagctgcc tcccatctca actccctgag agccacaggt |
| 1621 | gcctggagga gctgtgctgc cggaaaaagc cgggggcctg catcaccacc tcagagctgt |
| 1681 | tcaggaagct ggtcctgtcc agacacgtcc tgcagttcct cctgctctac caggagccct |
| 1741 | tgctggcgct ggatgtggat tccaccaaca gccggctgcg gcactgtgcc tacaggtgct |
| 1801 | acgccacctg gcgcttcggc tcccaggaca tggctgactt tgccatcctg cccagctgct |
| 1861 | gccgctggag gatccggaaa gagtttccga agagtgaagg gcagtacagt ggcttcaaga |
| 1921 | gtccttactg aagccaggca ccgtggctca cgtctgtaat cccagcgctt tgggaggccg |
| 1981 | aggcaggcag atcacctgag gtcgggagtt ggagacccgc ctggctaaca aggcgaaatc |
| 2041 | ctgtctgtac taaaaataca aaaatcagcc agacatggtg gcatgcacct gcaatcccag |
| 2101 | ctactcggga ggctgaggca caagaatcac ttgaacccgg gaggcagagg ttgtagtgag |
| 2161 | cccagattgt gccactgctc tccagcctgg gaggcacagc aaactgtccc ccaaaaaaaa |
| 2221 | aaaagagtcc ttaccaatag caggggctgc agtagccatg ttaacatgac atttaccagc |
| 2281 | aacttgaact tcacctgcaa agctctgtgg ccacattttc agccaaaggg aaatatgctt |
| 2341 | tcatcttctg ttgctctctg tgtctgagag caaagtgacc tggttaaaca aaccagaatc |
| 2401 | cctctacatg gactcagaga aaagagattg agatgtaagt ctcaactctg tccccaggaa |
| 2461 | gttgtgtgac cctaggcctc tcacctctgt gcctctgtct ccttgttgcc caactactat |
| 2521 | ctcagagata ttgtgaggac aaattgagac agtgcacatg aactgtcttt taatgtgtaa |
| 2581 | agatctacat gaatgcaaaa catttcatta tgaggtcaga ctaggataat gtccaactaa |
| 2641 | aaacaaaccc ttttcatcct ggctggagaa tgtggagaac taaaggtggc cacaaattct |
| 2701 | ttgacactca agtcccccaa gacctaaggg ttttatctcc tccccttgaa tatgggtggc |
| 2761 | tctgattgct ttatccaaaa gtggaagtga cattgtgtca gtttcagatc ctgatcttaa |
| 2821 | gaggctgaca gcttctactt gctgtccctt ggaactcttg ctatcgggga agccagacgc |
| 2881 | catttaaaag tctgcctatc ctggccaggt gtggtggctc acacctgtaa tcccagcact |
| 2941 | ttgggagacc aaggcgggcg gatcacttaa agtcaggagt ccaagaccag actcgccaac |
| 3001 | atggtgaaac cgtatctcta ataaaaatac aaaaattagc tgggcatggt gcgggcacct |
| 3061 | gtagtcctag ctatcaagag gctgagacag gagaaacact tgaacctggg aggtggaggt |
| 3121 | tgcattgagc tgagatcgtg ccactgcact ccaggctggg tgacagagcg agactccatc |
| 3181 | tcaaaaaaaa aaaaaagaaa aaaaaaatgt ctgcctatcc tgagactgcc ctgctgtgag |
| 3241 | gaagcccaag cagtcacgtg gacagtgcct gaccagcccc agctttcaag ccatccaagc |
| 3301 | ccagtcacca aacatgagag agaagaagcc ttcaggtgat tctggactcc actaacatat |
| 3361 | gactgatacc gcatgataca tcccaagtga gaactgcccc ataaatccag aaaaccacat |
| 3421 | tgctatctta agtccctaag tttggggctt atttgttcca cagcaacagg taactggaac |
| 3481 | agagggcaag cctgatgaat gggcacacag actcagccca taccttccct ggttctaatg |
| 3541 | ttctcaggga gcccggacca accctgggag cctcaggaac ttaggtttcc actggacagt |
| 3601 | tctagaaggg ctatagacca aatcaggtaa ctcaccagac cagccttgga atctatcaaa |
| 3661 | tctaactgct gagctaccca |

By “Cannabinoid receptor type 2 (CB2r) polypeptide” is meant a polypeptide or fragment thereof having at least about 85%, or greater, amino acid identity to NCBI Accession No.

NP_001832 (below) and having binding activity to tetrahydrocannabinol (THC) 2-Arachidonoylglycerol (2-AG), N-arachidonylethanolamide, anandamide, SR145528, AM320, and analogs thereof.

| | |
|---|---|
| 1 | meecwvteia ngskdgldsn pmkdymilsg pqktavavlc tllgllsale nvavlylils |
| 61 | shqlrrkpsy lfigslagad flasvvfacs fvnfhvfhgv dskavfllki gsvtmtftas |
| 121 | vgsllltaid rylclrypps ykalltrgra lvtlgimwvl salvsylplm gwtccprpcs |
| 181 | elfplipndy llswllfiaf lfsgiiytyg hvlwkahqhv aslsghqdrq vpgmarmrld |
| 241 | vrlaktlglv lavllicwfp vlalmahsla ttlsdqvkka fafcsmlcli nsmvnpviya |
| 301 | lrsgeirssa hhclahwkkc vrglgseake eaprssvtet eadgkitpwp dsrdldlsdc |

By “CNR2 nucleic acid molecule” is meant a polynucleotide encoding a CB2r polypeptide. An exemplary CB2r nucleic acid molecule sequence is provided at NCBI Accession No. NM_001841 (below):

| | |
|---|---|
| 1 | caggtcctgg gagaggacag aaaacaactg ggactcctca gcccccggca gctcccagtg |
| 61 | cccagccacc cacaacacaa cccaaagcct tctagacaag ctcagtggaa tctgaagggc |
| 121 | ccaccccatg gaggaatgct gggtgacaga gatagccaat ggctccaagg atggcttgga |
| 181 | ttccaaccct atgaaggatt acatgatcct gagtggtccc cagaagacag ctgttgctgt |
| 241 | gttgtgcact cttctgggcc tgctaagtgc cctggagaac gtggctgtgc tctatctgat |
| 301 | cctgtcctcc caccaactcc gccggaagcc ctcatacctg ttcattggca gcttggctgg |
| 361 | ggctgacttc ctggccagtg tggtctttgc atgcagcttt gtgaatttcc atgttttcca |
| 421 | tggtgtggat tccaaggctg tcttcctgct gaagattggc agcgtgacta tgaccttcac |
| 481 | agcctctgtg ggtagcctcc tgctgaccgc cattgaccga tacctctgcc tgcgctatcc |
| 541 | accttcctac aaagctctgc tcacccgtgg aagggcactg gtgaccctgg gcatcatgtg |
| 601 | ggtcctctca gcactagtct cctacctgcc cctcatggga tggacttgct gtcccaggcc |
| 661 | ctgctctgag cttttcccac tgatccccaa tgactacctg ctgagctggc tcctgttcat |
| 721 | cgccttcctc ttttccggaa tcatctacac ctatgggcat gttctctgga aggcccatca |
| 781 | gcatgtggcc agcttgtctg gccaccagga caggcaggtg ccaggaatgg cccgaatgag |
| 841 | gctggatgtg aggttggcca agaccctagg gctagtgttg gctgtgctcc tcatctgttg |
| 901 | gttcccagtg ctggccctca tggcccacag cctggccact acgctcagtg accaggtcaa |
| 961 | gaaggccttt gctttctgct ccatgctgtg cctcatcaac tccatggtca accctgtcat |
| 1021 | ctatgctcta cggagtggag agatccgctc ctctgcccat cactgcctgg ctcactggaa |
| 1081 | gaagtgtgtg aggggccttg ggtcagaggc aaaagaagaa gccccgagat cctcagtcac |
| 1141 | cgagacagag gctgatggga aaatcactcc gtggccagat tccagagatc tagacctctc |
| 1201 | tgattgctga tgaggcctct tcccaattta aacaactcaa gtcagaaatc agttcactcc |
| 1261 | ctggaagaga gagaggggtc ttggcactct cttcttactt aaaccagtcc cagacaccta |
| 1321 | gacacggacc cctttttgct gatgagtgtt gggactgact cctggaagac agcctggcct |
| 1381 | tgcccacctg cacacagtct gttggatagg tagggccacg aggagtagcc aggtaggcga |
| 1441 | gacacaaaag gcctgggaca gggtcagtac aagtcaggtc aggcttcatg cctgcatcct |
| 1501 | ccagagacca caggagccaa agcgagcctc caggcccagc aatgagggac ttgggagaaa |
| 1561 | tctgagaaga atgggttgtt ctcttgggaa gtcagggtat cagatgggat ggacatccag |
| 1621 | gtcttctctc tgcctaattg tcaaggcctc cttggctctg gagctatgaa aggccccact |
| 1681 | ttcaagtcac ccttgccact gaggaccgag gactatgcta tgatgaggat taaggtgttg |
| 1741 | acttgcctct ttcagagata aatgacaagc cttcaaaaaa aaaaaaaaa |

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

The term “antibody,” as used herein, refers to an immunoglobulin molecule which specifically binds with an antigen. The term “antibody fragment” refers to a portion of an intact antibody and refers to the antigenic determining variable regions of an intact antibody.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

By “capture reagent” or “binding agent” is meant a reagent that specifically binds a nucleic acid molecule or polypeptide to select or isolate the nucleic acid molecule or polypeptide. In various embodiments, the capture reagent is one or more of a small molecule, peptide, scFv, or aptamer that specifically binds a polypeptide marker of interest.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a protein or nucleic acid that is substantially identical to a reference protein or nucleic acid. In some embodiments the portion retains at least 50%, 75%, or 80%, or more preferably 90%, 95%, or even 99% of the biological activity of the reference protein or nucleic acid described herein.

By “inhibitory nucleic acid” or “inhibitory polynucleotide” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “detectable moiety” or “label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes, biotin, digoxigenin, or haptens.

By “marker” is meant any clinical indicator, protein, metabolite, or polynucleotide having an alteration associated with a disease, disorder, or condition. In various embodiments, the marker is a protein present on the surface of a cell, e.g., a receptor.

The term “monomer” or “building block” refers to any discreet chemical compound of any molecular weight. A monomer or building block may comprise two or more smaller monomers connected through chemical bonds, for example.

By “microglia” is meant an immune cell of the central nervous system.

By “nanoparticle” is meant a composite structure of nanoscale dimensions. In particular, nanoparticles are typically particles of a size in the range of from about 1 to about 500 nm, and are usually spherical although different morphologies are possible depending on the nanoparticle composition. The portion of the nanoparticle contacting an environment external to the nanoparticle is generally identified as the surface of the nanoparticle. In nanoparticles herein described, the size limitation can be restricted to two dimensions and so that nanoparticles herein described include composite structure having a diameter from about 1 to about 500 nm, where the specific diameter depends on the nanoparticle composition and on the intended use of the nanoparticle according to the experimental design. For example, nanoparticles to be used in several therapeutic and/or diagnostic applications typically have a size of about 200 nm or below, and the ones used, in particular, for delivery associated with therapeutic agents typically have a diameter from about 1 to about 100 nm.

As used herein “neurodegenerative disease” refers to any of a group of diseases characterized by the progressive loss of structure and/or function of neurons, including death of neurons. Exemplary neurodegenerative diseases include, without limitation, amyotrophic lateral sclerosis.

As used herein, the term “polymer” refers to a molecule composed of repeating structural units (or monomers) typically connected by covalent chemical bonds. The term “polymer” is also meant to include the terms copolymer and oligomers. In one embodiment, a polymer comprises a backbone (i.e., the chemical connectivity that defines the central chain of the polymer, including chemical linkages among the various polymerized monomeric units) and a side chain (i.e., the chemical connectivity that extends away from the backbone).

As used herein, the term “polymerization” or “crosslinking” refers to at least one reaction that consumes at least one functional group in a monomeric molecule (or monomer), oligomeric molecule (or oligomer) or polymeric molecule (or polymer), to create at least one chemical linkage between at least two distinct molecules (e.g., intermolecular bond), at least one chemical linkage within the same molecule (e.g., intramolecular bond), or any combinations thereof. A polymerization or crosslinking reaction may consume between about 0% and about 100% of the at least one functional group available in the system. In one embodiment, polymerization or crosslinking of at least one functional group results in about 100% consumption of the at least one functional group. In another embodiment, polymerization or crosslinking of at least one functional group results in less than about 100% consumption of the at least one functional group. Polymerization reactions comprise, for example, ether formation, thioether formation, thioester formation, ester formation and amide formation.

A “copolymer” is a polymer derived from two or more monomeric species (or monomers or building blocks). Copolymerization refers to methods used to chemically synthesize a copolymer. Copolymers vary depending on the different types and arrangement of monomers. For example, in a copolymer consisting of two different types of monomers, the copolymers may be alternating (wherein the two different types of monomers alternate on the copolymer), block (wherein the copolymer comprises two or more homopolymers linked by covalent units), periodic (wherein a specific sequence of the two types of monomers repeats itself throughout the copolymer), or statistical (wherein the sequence of monomers follows a statistical rule).

By “increasing proliferation” is meant increasing cell division of a cell in vivo or in vitro.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

By “reduces” is meant a negative alteration of at least 10%, 25%, 50%, 75%, or 100%.

By “reference” is meant a standard of comparison or control condition.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95%, 96%, 97%, 98%, or even 99% or more identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰° indicating a closely related sequence.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “specifically binds” is meant a compound (e.g., peptide) that recognizes and binds a molecule (e.g., polypeptide), but which does not substantially recognize and bind other molecules in a sample, for example, a biological sample.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### BRIEF DESCRIPTION OF THE DRAWINGS

FIG. 1 depicts cell targeted functionalized polymers and cell targeted functionalized drug-loaded nanoparticles and their uses in therapeutic drug delivery and/or diagnostics.

FIGS. 2A-2F depict monomers and block-copolymers generated in different steps to obtain MPC-based nanoparticles (NPs) with optimal dimensions, polyspersion, and suitable for functionalization for targeted applications. FIG. 2A depicts the structure of methacryloyloxyethyl phosphorylcholine (MPC). FIG. 2B depicts the structures of NH₂-Peg-Boc and MePeg-BOC. FIG. 2C depicts the structure of nMPC-mMePegBoc. FIG. 2D depicts the structure of block copolymer of nMPC mMePegBoc qPCL5Q. FIG. 2E depicts Dynamic Light Scattering (DLS) analysis of methacryloyloxyethyl phosphorylcholine nanoparticles (NP-MPC). FIG. 2F depicts functional group (Peg-N3) can be used for ligand functionalization through click-chemistry in place of Peg-Boc in the structures depicted in FIGS. 2B-2D. In FIGS. 2B-2D and 2F, the box highlights the functional group utilized for ligand functionalization (Peg-Boc)

FIGS. 3A-3C depicts uptake of NP-MPC assessed in vitro and in vivo. FIG. 3A depicts uptake of NP50 or NP-MPC in BV2 microglia cell lines, assessed by flow cytometry. FIG. 3B depicts flow cytometric evaluation of NP50 or NP-MPC biodistribution in the brain of mice, upon injection in the cerebral lateral ventricles (ICV) or intrathecally at lumbar level (ITL). FIG. 3C depicts flow cytometric evaluation of NP50 or NP-MPC biodistribution in the spinal cord of mice upon injection in the cerebral lateral ventricles (ICV) or intrathecally at lumbar level (ITL). FIG. 3D depicts cytofluorimetric quantification of the biodistribution of NP-MPC within different CNS cell types: CD45⁺ versus CD45⁻. FIG. 3E depicts cytofluorimetric quantification of the biodistribution of NP-MPC within different CNS cell types: CD11b⁺ microglia versus CD11b⁻ leukocytes within CD45⁺ fraction. FIG. 3F depicts cytofluorimetric quantification of the biodistribution of NP-MPC within different CNS cell types: O4⁺ oligodendrocytes versus ACSA2⁺ astrocytes, CD31⁺ vascular endothelial cells or Thy1.2 positive neurons, within CD45⁻ fraction. FIG. 3G depicts the brain biodistribution of NP-MPC upon intrathecal injection at lumbar level (ITL).

FIG. 4A depicts a schematic representation of the steps for production of a block copolymer capable of loading iron in NPs (top panel). FIG. 4B is a series of graphs illustrating the dynamic light scattering analysis of MPC-NPs before and after loading of iron. FIG. 4C outlines the main features of the polymers used to prepare SPIONs 1-3, SPIONs 4-7 and SPIONs 8-9. FIG. 4D depicts representative brightfield microscopy microphotographs and corresponding quantification of iron content BV2 cells exposed to different SPION batches (tested at 0.2 mg polymer/ml). FIG. 4E is a graph quantifying the results observed in FIG. 4D. FIG. 4F is a graph depicting rhodamine signal detected by flow cytometry in BV2 cells exposed to different concentrations of fluorescent labelled SPION batches numbered SPION 4 through 9.

FIGS. 5A-5B depict NP biodistribution in vivo by MRI. FIG. 5A depicts T2 (spin-spin) and SWI (susceptibility weighted imaging) MRI images of the brain of a wild type or a SOD1.G93A mouse. The latter was injected bilaterally ICV (SOD1.G93A, arrows in upper left panel) with NP-SPION. Hyper-intense signal was detected in the facial nucleus of SOD1.G93A mouse (SOD1.G93A, arrows in upper right panel), consistent with the neurodegeneration occurring at this disease stage. Prominent NPs+ signal (arrow) was detected by SWI at the site of injection and throughout the brain parenchyma (SOD1.G93A, arrows in bottom panels). FIG. 5B depicts cytofluorimetric analysis of rhodaminated NP-SPION biodistribution, confirming the uptake by CNS CD45⁺/CD11b⁺ microglia cells.

FIG. 6A depicts functionalization of Hema-succinate with deferoxamine (DFO), to form Hema-DFO, a building block used to produce NP-MPC functionalized on the surface with DFO. FIG. 6B depicts dynamic light scattering (DLS) analysis of DFO-NPs. FIG. 6C depicts the binding profile of NP-MPC functionalized with DFO for the radioisotope ⁸⁹Zr. FIG. 6D depicts the internalization of rhodaminated NP-MPC functionalized with DFO (assessed by flow cytometry) in microglia cells from mouse brain and spinal cord.

FIG. 7A depicts synthesis of the poly-caprolactone-based diacrylate. FIG. 7B depicts synthesis of Poly (β-amino ester) (PBAE) terpolymers. FIG. 7C depicts synthesis of hyperbranched PBAEs.

FIG. 8A is a graph depicting the DNA transfection efficiency of different PBAEs (1 to 16) loaded on the zwitterionic block copolymer MPC (expressed as a percentage of cells positive for the fluorescent reporter, in comparison with a reference commercial liposomic mixture “Ref”), assessed in BV2 microglia cell line by flow cytometry 48 hours after treatment. FIG. 8B is a graph depicting cellular DNA transfection efficiency of different PBAEs (1 to 16) loaded on the zwitterionic block copolymer MPC (expressed as intensity of the fluorescent reporter, corresponding to the extent of gene expression, in comparison with a reference commercial liposomic mixture “Ref”), assessed in BV2 microglia cell line by flow cytometry 48 hr after treatment. FIG. 8C depicts the distribution of GFP reporter gene in BV2 microglia cell line, assessed 16 hours and 48 hours post-transfection with PBAE polymer 12 complexed with a GFP-expressing plasmidic DNA and encapsulated in the zwitterionic block copolymer MPC. Brightfield shows the morphology of BV2 cells in the plate; Rh shows the rhodamine-positive signal, corresponding to the internalization of rhodaminated MPC NPs; GFP shows the signal of the reporter gene expressed by the plasmid upon internalization in the cells; Merge pictures show the merging of brightfield, Rh and GFP channels.

FIG. 9A depicts the molecular formula of the TSPO ligand PK11195 and its precursor 1-(2-chlorophenyl)isoquinoline-3-carboxylic acid (ClPhIQ). FIG. 9B depicts the molecular formula of the TSPO ligand PBR28 and the precursor (PBR28-click reactant).

FIG. 10A depicts 1-(2-chlorophenyl)isoquinoline-3-carboxylic acid bound with primary amine. The circle highlights the bond with one of the primary amines present on the terminal branches of the nanoparticles. The box highlights the carbonyl group, present also in the formula of PK11195, which is involved in important interactions with the TSPO receptor binding pocket. FIG. 10B is a schematic structure of the final polymer, carrying both TSPO-selective ligands and fluorescent dyes. The molar ratios of ligand/dye and the length of the side chains (arrow) can be modified, allowing the ability to explore different nanomaterial configurations. FIG. 10C depicts H-NMR analysis of a ClPhIQ-polymer conjugate used to confirm the loading of ClPhIQ on the comb-like nanoparticles.

FIG. 11A depicts assays to test the uptake of the PK11195 functionalized polymers P1F and the corresponding non-functionalized counterpart NF-P1 in BV2 microglia cell line. FIG. 11B depicts assays to test the uptake of the PK11195 functionalized polymers P3F and the corresponding non-functionalized counterpart NF-P3 in BV2 microglia cell line. FIG. 11C depicts assays to test the uptake of the PK11195 functionalized polymers P5F and the corresponding non-functionalized counterpart NF-P5 in BV2 microglia cell line. FIG. 11D depicts assays to test the uptake of the PK11195 functionalized polymers P7F and the corresponding non-functionalized counterpart NF-P7 in BV2 microglia cell line. For FIGS. 11A to 11D, the top graphs illustrate the presence of functionalized and nonfunctionalized labeled nanoparticles (P1, P3, P5, and P7) with cells to which the NPs were administered as measured by fluorescence. The bottom left graph shows flow cytometry results for cells treated with different concentrations of functionalized nanoparticles, non-functionalized nanoparticles, and untreated cells. The bottom right panel is a graph depicting the results from competition assays between the functionalized polymer and unconjugated ligand.

FIG. 12A is a graph showing uptake of the functionalized polymers P1F, P3F, P5F, and P7F in BV2 cell line exposed to LPS stimulation (1 μg/ml). FIG. 12B depicts representative laser scanning confocal microphotographs of FITC⁺ functionalized (P3F, P5F) and the corresponding non-functionalized (3NF, 5NF) polymers (gray), TSPO (light gray) and DAPI nuclear staining (dark gray) in BV2 cells. No FITC⁺ signal was detected in the cells exposed to the non-functionalized polymers, whereas BV2 cells treated with P3F or P5F display prominent FITC⁺ punctate signal in the cytoplasm, which was partially co-localized with TSPO staining. FIG. 12C are graphs illustrating the different fractions of Cy5^{high} cells and the percentage of Cy5^{high} cells in a population of cells after treatment with PK11195.

FIGS. 13A-13B depict flow cytometric evaluation of the uptake of polymers P3 and P5 (functionalized and non-functionalized) on BV2 cells lines expressing physiological levels (#scramble cell line) or about 20% of normal levels (#106 cell line) of TSPO. FIG. 13A depicts assessment performed after 4 hours of incubation of the polymers on cells. ****=p<0.001; 2-way ANOVA followed by Tukey's post-hoc test (n≥3). FIG. 13B depicts assessment performed after 24 hours of incubation of the polymers on cells. ****=p<0.001; 2-way ANOVA followed by Tukey's post-hoc test (n≥3).

FIGS. 14A-14E depict binding as assessed by microscale thermophoresis (MST). FIG. 14A depicts normalized fluorescence intensity (Fnorm) evaluated for red fluorescent tagged hTSPO alone or in the presence of PK11195. FIG. 14B depicts normalized fluorescence intensity (Fnorm) evaluated for red fluorescent tagged hTSPO alone or in the presence of Ro5-4864. FIG. 14C depicts MST traces evaluated for non-functionalized polymers. FIG. 14D depicts MST traces evaluated for PK11195-functionalized polymer. FIG. 14E depicts quantification of baseline normalized fluorescence intensity for the traces shown in FIGS. 14C and 14D and highlights binding of P3F and P5F to hTSPO target. ****=p<0.001; 1-way ANOVA followed by Dunnet's post-hoc test vs No ligand (n≥3).

FIG. 15A is a graph depicting the relative release profile of 25MPC5CL3-BCL3 and 25MPC80CL5 NPs loaded with Diapocynin. FIG. 15B depicts the inhibitory effect of dyapocinin on reactive oxygen species (ROS) production induced in BV2 cells by exposure to phorbol-ester myristate acetate (PMA). The effect of the exposure on diphenylene iodonium (DPI) is shown as reference compound. FIG. 15C depicts the inhibitory effects of dyapocinin encapsulated in MPC-NPs on PMA-induced ROS production in BV2 cells. *=p<0.05; ***=p<0.001; ****=p<0.00001; ANOVA followed by Tukey's post-hoc test.

FIGS. 16A-16D depict flow cytometric evaluation of the uptake of NP-MPC functionalized with PBR-28 precursor (MPC-PBR) and non-functionalized (MPC-NF) after 4 hours or 24 hours of incubation in BV2 cells. FIG. 16A is a graph illustrating the uptake of batch 2_1 NP-MPC functionalized with PBR-28 precursor (MPC-PBR) and non-functionalized (MPC-NF) after 4 hours or 24 hours of incubation in BV2 cells. FIG. 16B is a graph illustrating the uptake of batch 2_2 NP-MPC functionalized with PBR-28 precursor (MPC-PBR) and non-functionalized (MPC-NF) after 4 hours or 24 hours of incubation in BV2 cells. FIG. 16C is a graph illustrating the uptake of batch 1_1 NP-MPC functionalized with PBR-28 precursor (MPC-PBR) and non-functionalized (MPC-NF) after 4 hours or 24 hours of incubation in BV2 cells. 10 FIG. 16D is a graph illustrating the uptake of batch 1_2 NP-MPC functionalized with PBR-28 precursor (MPC-PBR) and non-functionalized (MPC-NF) after 4 hours or 24 hours of incubation in BV2 cells. Table 4 provides a summary of MPC-PBR and MPC-NF batches. FIGS. 16E-16H depict a competition assay: flow cytometric evaluation of the uptake of MPC-PBR or MPC-NF in BV2 cells after incubation for 4 hours in the presence of increasing concentrations of the free ligand PBR-28. FIG. 16E is a graph illustrating the uptake of batch 2_1 MPC-PBR or MPC-NF in BV2 cells after incubation for 4 hours in the presence of increasing concentrations of the free ligand PBR-28. FIG. 16F is a graph illustrating the uptake of batch 2_2 MPC-PBR or MPC-NF in BV2 cells after incubation for 4 hours in the presence of increasing concentrations of the free ligand PBR-28. FIG. 16G is a graph illustrating the uptake of batch 1_1 MPC-PBR or MPC-NF in BV2 cells after incubation for 4 hours in the presence of increasing concentrations of the free ligand PBR-28. FIG. 16H is a graph illustrating the uptake of batch 1_2 MPC-PBR or MPC-NF in BV2 cells after incubation for 4 hours in the presence of increasing concentrations of the free ligand PBR-28. Values are shown as percentage to the uptake reported in the untreated controls (UT, i.e. no PBR-28 in the culture medium). ***=p<0.001; **=p<0.01; *=p<0.05; 2-way ANOVA followed by Dunnet's post-hoc test versus respective UT (n>3).

FIGS. 17A-17B depict flow cytometric evaluation of the uptake of NP-MPC, either functionalized with PBR-28 precursor (MPC-PBR) or not (MPC-NF) on BV2 cells lines expressing physiological levels (#scramble cell line) or about 20% of normal levels (#106 cell line) of TSPO. FIG. 17A depicts assessment performed after 4 hours of incubation of the NPs on cells. 2-way ANOVA (n>3). FIG. 17B depicts assessment performed after 24 hours of incubation of the NPs on cells. 2-way ANOVA (n>3).

FIG. 18 depicts ten different PK11195 precursors, carrying an alkine group at variable distance from the site of interaction with the receptor, highlighted by the dashed box.

FIG. 19 depicts seven different PBR28 precursors, carrying an alkine group at variable distance from the site of interaction with the receptor, highlighted by the dashed box.

FIG. 20A depicts the progressive increase of the percentage of microglia cells stained positive for TSPO detected by flow cytometry in the brain and spinal cord of SOD1.G93A mouse model of ALS as the disease worsens. FIG. 20B depicts representative laser scanning confocal microscope images of reactive CD11⁺ microglia cells showing increased staining for TSPO in the lumbar spinal cord of symptomatic SOD1.G93A mouse model of ALS (arrows in FIG. 20B). FIG. 20C depicts the extent of internalization of MPC-PBR 2_2 (MPC-NPs functionalized with a PBR28 precursor) or the corresponding not functionalized NPs, namely MPC-NF 2_2, in microglia cells retrieved from the brain of SOD1.G93A mouse model of ALS 3 days after NPs administration at lumbar level. The percentage of cells positive for TSPO (receptor for PBR28) within the rhodamine positive fraction is also depicted. *=p<0.05; Mann-Whitney test. FIG. 20D depicts the extent of internalization of MPC-PBR 2_2 or MPC-NF 2_2 in microglia cells retrieved from the spinal cord of SOD1.G93A mouse model of ALS 3 days after NPs administration at lumbar level. The percentage of cells positive for TSPO within the rhodamine positive fraction is also depicted.

### DETAILED DESCRIPTION OF THE INVENTION

As described herein, the present invention features compositions and methods for targeting and delivery of therapeutic and/or diagnostic agents to a cell. This invention is directed to an innovative pharmacological platform that can provide cell-target specificity and the possibility to monitor non-invasively the drug biodistribution and the therapeutic efficacy in vivo. The invention includes the use of nanoparticles and polymers functionalized with capture molecules, reporter molecules, and/or therapeutic agents for the treatment or prevention of disease, including neurological diseases associated with neuroinflammation and cancer.

The present invention is based at least in part on several discoveries described herein. Briefly, the innovation comprises the use of polymeric nanoparticles functionalized with cell-target selective ligands, combining, at the same time, the capability of. i) cell-specific delivery of therapeutic molecules, with potential improvement in drug efficacy and prevention of possible unwanted side effects; ii) controlled release of multiple drugs, thus widening the spectrum of key molecular pathways that can be targeted at the same time (an aspect of great value for multi-factorial diseases such as Amyotrophic Lateral Sclerosis (ALS) and other neurodegenerative diseases where neuroinflammation is involved); and/or iii) providing prognostic/diagnostic information (by identification of the CNS areas affected by neuroinflammation) and monitoring the sites of drug delivery.

This approach reflects principles of precision medicine, an advancing field of science that holds great promise for ALS and neurodegenerative diseases. In contrast to the therapeutic approaches tested up to now, that were mainly based on chronic administration of neuroprotective factors (e.g. trophic or anti-apopotic factors, treatment with anti-glutamatergic drugs or compounds enhancing proteasome or mitochondrial metabolic activity) or anti-inflammatory molecules (such as cyclooxygenase inhibitors or minocycline) among others. These strategies suffered of limited efficacy, due to possible induction of pharmacological tolerance or to the lack of specificity for selective cell types (glia versus neurons or neurotoxic versus neuro-supportive microglia). Moreover, they do not take into account the complexity of the CNS pathology, where in different CNS districts we can observe compensatory neuro-supportive attempts that co-exist with, or eventually evolve to, pro-degenerative cellular and molecular responses. In fact, as an example, the most common initial presentation of ALS is focal asymmetric distal weakness accompanied by muscular atrophy, which reflects the presence of specific foci of neuronal dysfunction in restricted CNS areas. Like in any biological system, the organism senses these damages and initially tries to compensate the neuronal loss (e.g., compensatory reinnervation from nearby motor neurons permits a good maintenance of the motor function until more than 50% of motor units have been lost). However, over-time this scenario eventually worsens through spreading of the damage to the other neuronal districts, finally causing the progressive deterioration of motor function which eventually leads to a fatal outcome. The response of microglia cells in ALS fits perfectly in this picture: microglia activation initially exerts a neuro-supportive function but eventually this response results insufficient and is overwhelmed by a shift towards a more neuro-toxic phenotype.

### Neurodegenerative Diseases

Neurodegenerative diseases are a class of neurological diseases that are characterized by the progressive loss of the structure and/or function of neurons and/or neuronal cell death. Inflammation has been implicated for a role in several neurodegenerative diseases. Progressive loss of motor and sensory neurons and the ability of the mind to refer sensory information to an external object is affected in different kinds of neurodegenerative diseases. Non-limiting examples of neurodegenerative diseases include ALS, e.g., familial ALS and sporadic ALS.

Relationships between microglia and neurodegeneration have been observed. Activation of glial cells in ALS plays an important role in disease progression and spreading of the pathology to other CNS districts. Aberrant activation of microglia cells in ALS orchestrates a neurotoxic environment.

A health care professional may diagnose a subject as having a neurodegenerative disease by the assessment of one or more symptoms of a neurodegenerative disease in the subject. Non-limiting symptoms of a neurodegenerative disease in a subject include difficulty lifting the front part of the foot and toes; weakness in arms, legs, feet, or ankles; hand weakness or clumsiness; slurring of speech; difficulty swallowing; muscle cramps; twitching in arms, shoulders, and tongue; difficulty chewing; difficulty breathing; muscle paralysis; partial or complete loss of vision; double vision; tingling or pain in parts of body; electric shock sensations that occur with head movements; tremor; unsteady gait; fatigue; dizziness; loss of memory; disorientation; misinterpretation of spatial relationships; difficulty reading or writing; difficulty concentrating and thinking; difficulty making judgments and decisions; difficulty planning and performing familiar tasks; depression; anxiety; social withdrawal; mood swings; irritability; aggressiveness; changes in sleeping habits; wandering; dementia; loss of automatic movements; impaired posture and balance; rigid muscles; bradykinesia; slow or abnormal eye movements; involuntary jerking or writhing movements (chorea); involuntary, sustained contracture of muscles (dystonia); lack of flexibility; lack of impulse control; and changes in appetite. A health care professional may also base a diagnosis, in part, on the subject's family history of a neurodegenerative disease. A health care professional may diagnose a subject as having a neurodegenerative disease upon presentation of a subject to a health care facility (e.g., a clinic or a hospital). In some instances, a health care professional may diagnose a subject as having a neurodegenerative disease while the subject is admitted in an assisted care facility. Typically, a physician diagnoses a neurodegenerative disease in a subject after the presentation of one or more symptoms.

### Nanoparticles

The present invention provides methods of delivering nanoparticles comprising a capture reagent, a therapeutic agent, cytotoxic agent (e.g., cell ablation), and/or a detectable reporter comprising administering a nanoparticle comprising the one or more agents to a subject (e.g., a mammal such as a human).

In general, a “nanoparticle” refers to any particle having a diameter of less than 500 nm. In certain preferred embodiments, nanoparticles of the invention have a greatest dimension (e.g., diameter) of 200 nm or less. In other preferred embodiments, nanoparticles of the invention have a greatest dimension ranging between 25 nm and 100 nm. In other preferred embodiments, nanoparticles of the invention have a greatest dimension of 100 nm or less. In other preferred embodiments, nanoparticles of the invention have a greatest dimension ranging between about 45 nm and 50 nm. Nanoparticles encompassed in the present invention may be provided in different forms, e.g., as solid nanoparticles (e.g., non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Such nanoscale particles are used in biomedical applications as drug carriers or imaging agents and may be adapted for similar purposes in the present invention. Semi-solid and soft nanoparticles have been manufactured, and are within the scope of the present invention. Nanoparticles with surfaces that are half hydrophilic and half hydrophobic are termed Janus particles and are particularly effective for stabilizing emulsions. They can self-assemble at water/oil interfaces and act as solid surfactants. In one embodiment, nanoparticles based on self assembling bioadhesive polymers are contemplated, which may be applied to oral delivery of agents, intravenous delivery of agents and nasal delivery of agents, all to the brain. Other embodiments, such as oral absorption of hydrophobic drugs, are also contemplated. The molecular envelope technology involves an engineered polymer envelope that is protected and delivered to the site of the disease (Mazza et al. ACS Nano 7, 1016-1026 (2013); Siew et al. Mol Pharm 9, 14-28 (2012); Lalatsa et al. J Control Release 161, 523-536 (2012); Lalatsa et al. Mol Pharm 9, 1665-1680 (2012); Garrett et al. J Biophotonics 5, 458-468 (2012); Uchegbu, Expert Opin Drug Deliv 3, 629-640 (2006); Uchegbu et al. Int J Pharm 224, 185-199 (2001); Qu et al. Biomacromolecules 7, 3452-3459 (2006)).

Several types of particle delivery systems and/or formulations are known to be useful in a diverse spectrum of biomedical applications. In general, a particle is defined as a small object that behaves as a whole unit with respect to its transport and properties. Particles are further classified according to diameter. Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm.

As used herein, a particle delivery system/formulation is defined as any biological delivery system/formulation, which includes a particle in accordance with the present invention. A particle in accordance with the present invention is any entity having a greatest dimension (e.g. diameter) of less than 500 nm. In some embodiments, inventive particles have a greatest dimension of less than 200 nm. In some embodiments, inventive particles have a greatest dimension of less than 100 nm. In some embodiments, inventive particles have a greatest dimension of less than 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarisation interferometry, microscale thermophoresis (MST), and nuclear magnetic resonance (NMR). Characterization (dimension measurements) may be made as to native particles (i.e., preloading) or after loading of the cargo (therapeutic agents, detectable reporters, or any combination thereof, and may include additional carriers and/or excipients) to provide particles of an optimal size for delivery for any in vitro, ex vivo, and/or in vivo application of the present invention. In certain preferred embodiments, particle dimension (e.g., diameter) characterization is based on measurements using dynamic laser scattering (DLS).

Particle delivery systems within the scope of the present invention may be provided in any form, including but not limited to solid, semi-solid, emulsion, or colloidal particles. As such any of the delivery systems described herein may be provided as particle delivery systems within the scope of the present invention.

### Methods of Treatment

The present invention provides methods of treating disease and/or disorders or symptoms thereof which comprise administering a therapeutically effective amount of a pharmaceutical composition comprising a nanoparticle or polymer described herein to a subject (e.g., a mammal such as a human). In certain embodiments, the subject has a neurological disease, e.g., associated with neuroinflammation, and the nanoparticle or polymer is used to deliver a therapeutic agent, e.g., diapocynin, to a cell in the CNS, e.g., a microglia. In other embodiments, the subject has cancer or a neoplasia, and the nanoparticle or polymer is used to deliver a chemotherapeutic and/or cytotoxic agent, e.g., etoposide or lomustine, to a cancer cell. Thus, in various embodiments the invention is directed to methods of treating a subject suffering from or susceptible to a disease or disorder or symptom thereof. The method includes the step of administering to the mammal a therapeutic amount of a nanoparticle or polymer herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method). Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). In some embodiments, the compounds herein are also used in the treatment of any other disorders in which myelination deficiency or loss is implicated, including multiple sclerosis.

### Compounds and Synthesis

The compounds of the invention can be prepared from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds described herein.

Synthetic chemistry transformations (including protecting group methodologies) useful in synthesizing the compounds described herein are known in the art and include, for example, those such as described in R. C. Larock, Comprehensive Organic Transformations, 2d.ed., Wiley-VCH Publishers (1999); P. G. M. Wuts and T. W. Greene, Protective Groups in Organic Synthesis, 4th Ed., John Wiley and Sons (2007); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The processes described herein can be monitored according to any suitable method known in the art. For example, product formation can be monitored by spectroscopic means, such as nuclear magnetic resonance spectroscopy (e.g., ¹H or ¹³C), infrared spectroscopy (FT-IR), spectrophotometry (e.g., UV-visible), or mass spectrometry (MS), or by chromatography such as high performance liquid chromatography (HPLC) or thin layer chromatography (TLC).

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Resolution of racemic mixtures of compounds can be carried out by any of numerous methods known in the art. An example method includes preparation of the Mosher's ester or amide derivative of the corresponding alcohol or amine, respectively. The absolute configuration of the ester or amide is then determined by proton and/or ¹⁹F NMR spectroscopy. An example method includes fractional recrystallization using a “chiral resolving acid,” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid, or the various optically active camphorsulfonic acids. Resolution of racemic mixtures can also be carried out by elution on a column packed with an optically active resolving agent (e.g., dinitrobenzoylphenylglycine). Suitable elution solvent compositions can be determined by one skilled in the art.

### Antibodies

As reported herein, antibodies that specifically bind a marker (e.g., a cell surface moiety or receptor) are useful in the methods of the invention, including therapeutic and diagnostic methods. In particular embodiments, the invention provides a nanoparticle or polymer having an scFv or antibody fragment that specifically binds a cell surface marker of a microglia and contains a therapeutic and/or diagnostic agent.

Antibodies can be intact immunoglobulins derived from natural sources or from recombinant sources and can be immunoreactive portions of intact immunoglobulins. Antibodies are typically tetramers of immunoglobulin molecules. Tetramers may be naturally occurring or reconstructed from single chain antibodies or antibody fragments. As used herein, the term “antibody” means not only intact antibody molecules, but also fragments of antibody molecules that retain immunogen-binding ability. Such fragments are also well known in the art and are regularly employed both in vitro and in vivo. Examples of antibody fragments include, but are not limited to, Fab, Fab′, F(ab′) 2, and Fv fragments, linear antibodies, scFv antibodies, single-domain antibodies, such as camelid antibodies (Riechmann, 1999, Journal of Immunological Methods 231:25-38), composed of either a VL or a VH domain which exhibit sufficient affinity for the target, and multispecific antibodies formed from antibody fragments.

The antibodies in the present invention may exist in a variety of forms including, for example, polyclonal antibodies, monoclonal antibodies, Fv, Fab and F(ab′)2, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). For example, F(ab′)₂, and Fab fragments that lack the Fc fragment of an intact antibody, clear more rapidly from the circulation, and may have less non-specific tissue binding than an intact antibody (Wahl et al., J. Nucl. Med. 24:316-325 (1983). Thus, the antibodies of the invention comprise, without limitation, whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8(10): 1057-1062,1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of ˜60-100 kDa in size provide faster blood clearance and rapid tissue uptake—See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making and using unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments using single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

In various embodiments, an antibody is monoclonal. Alternatively, the antibody is a polyclonal antibody. The preparation and use of polyclonal antibodies are also known by the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Methods of preparing antibodies are well known to those of ordinary skill in the science of immunology. Antibodies can be made by any of the methods known in the art utilizing a soluble polypeptide, or immunogenic fragment thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding polypeptides or immunogenic fragments thereof, can be provided to the host in a delivery vehicle that is taken up by immune cells of the host. The cells will in turn express the polypeptide thereby generating an immunogenic response in the host. Alternatively, nucleic acid sequences encoding human polypeptides or immunogenic fragments thereof, can be expressed in cells in vitro, followed by isolation of the polypeptide and administration of the polypeptide to a suitable host in which antibodies are raised.

Alternatively, antibodies may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

### Aptamers

Aptamers are another class of binding agent or capture reagent that can be used to target the compositions of the invention to a cell. Aptamers are nucleic acid-based molecules that bind specific ligands. Aptamers that specifically bind a marker of the cell (e.g., a cell surface moiety or receptor) are useful in the methods of the invention. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

### Recombinant Polypeptide Expression

The nanoparticle and polymer compositions of the invention can be functionalized with polypeptide capture molecules and detectable reporters. To express the polypeptides of the invention, DNA molecules obtained by any of the methods described herein or those that are known in the art, can be inserted into appropriate expression vectors by techniques well-known in the art. For example, a double stranded DNA can be cloned into a suitable vector by restriction enzyme linking involving the use of synthetic DNA linkers or by blunt-ended ligation. DNA ligases are usually used to ligate DNA molecules and undesirable joining can be avoided by treatment with alkaline phosphatase.

Therefore, the invention includes vectors (e.g., recombinant plasmids) that include nucleic acid molecules (e.g., genes or recombinant nucleic acid molecules encoding genes) as described herein. The term “recombinant vector” includes a vector (e.g., plasmid, phage, phasmid, virus, cosmid, fosmid, or other purified nucleic acid vector) that has been altered, modified or engineered such that it contains greater, fewer, or different nucleic acid sequences than those included in the native or natural nucleic acid molecule from which the recombinant vector was derived. For example, a recombinant vector may include a nucleotide sequence encoding a polypeptide, or fragment thereof, operatively linked to regulatory sequences, e.g., promoter sequences, terminator sequences, and the like, as defined herein. Recombinant vectors which allow for expression of the genes or nucleic acids included in them are referred to as “expression vectors.”

In some of the molecules of the invention described herein, one or more DNA molecules having a nucleotide sequence encoding one or more polypeptides of the invention are operatively linked to one or more regulatory sequences, which are capable of integrating the desired DNA molecule into a prokaryotic host cell. Cells which have been stably transformed by the introduced DNA can be selected, for example, by introducing one or more markers which allow for selection of host cells which contain the expression vector. A selectable marker gene can either be linked directly to a nucleic acid sequence to be expressed, or be introduced into the same cell by co-transfection. Additional elements may also be needed for optimal synthesis of proteins described herein. It would be apparent to one of ordinary skill in the art which additional elements to use.

Factors of importance in selecting a particular plasmid or viral vector include, but are not limited to, the ease with which recipient cells that contain the vector are recognized and selected from those recipient cells which do not contain the vector; the number of copies of the vector which are desired in a particular host; and whether it is desirable to be able to “shuttle” the vector between host cells of different species.

Once the vector(s) is constructed to include a DNA sequence for expression, it may be introduced into an appropriate host cell by one or more of a variety of suitable methods that are known in the art, including but not limited to, for example, transformation, transfection, conjugation, protoplast fusion, electroporation, calcium phosphate-precipitation, direct microinjection, etc.

After the introduction of one or more vector(s), host cells are usually grown in a selective medium, which selects for the growth of vector-containing cells. Expression of recombinant proteins can be detected by immunoassays including Western blot analysis, immunoblot, and immunofluorescence. Purification of recombinant proteins can be carried out by any of the methods known in the art or described herein, for example, any conventional procedures involving extraction, precipitation, chromatography, and electrophoresis. A further purification procedure that may be used for purifying proteins is affinity chromatography using monoclonal antibodies which bind a target protein. Generally, crude preparations containing a recombinant protein are passed through a column on which a suitable monoclonal antibody is immobilized. The protein usually binds to the column via the specific antibody while impurities pass through. After washing the column, the protein is eluted from the gel by changing pH or ionic strength, for example.

The nanoparticle and polymer compositions of the invention can also be used to achieve gene transfer. cDNA expression for use in polynucleotide therapy methods can be directed from any suitable promoter (e.g., the human cytomegalovirus (CMV) promoter), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

### Methods for Evaluating Therapeutic Efficacy

In one approach, the efficacy of the treatment is evaluated by measuring, for example, the biological function of the treated organ (e.g., neuronal function). Such methods are standard in the art and are described, for example, in the Textbook of Medical Physiology, Tenth edition, (Guyton et al., W. B. Saunders Co., 2000). In particular, a method of the present invention, increases the biological function of a tissue or organ by at least 5%, 10%, 20%, 40%, 50%, 60%, 70%, 80%, 90%, 100%, 150%, 200%, or even by as much as 300%, 400%, or 500%. In one embodiment, the tissue is neuronal tissue.

In another approach, the therapeutic efficacy of the methods of the invention is assayed by measuring an increase or decrease in cell number in the treated tissue or organ as compared to a corresponding control tissue or organ (e.g., a tissue or organ that did not receive treatment). Preferably, cell number in a tissue or organ is increased or decreased, depending on the disease, by at least 5%, 10%, 20%, 40%, 60%, 80%, 100%, 150%, or 200% relative to a corresponding tissue or organ. Methods for assaying cell proliferation are known to the skilled artisan and are described, for example, in Bonifacino et al., (Current Protocols in Cell Biology Loose-leaf, John Wiley and Sons, Inc., San Francisco, Calif.). For example, assays for cell proliferation may involve the measurement of DNA synthesis during cell replication. In one embodiment, DNA synthesis is detected using labeled DNA precursors, such as [^{3H}]-thymidine or 5-bromo-2*-deoxyuridine [BrdU], which are added to cells (or animals) and then the incorporation of these precursors into genomic DNA during the S phase of the cell cycle (replication) is detected (Ruefli-Brasse et al., Science 302(5650):1581-4, 2003; Gu et al., Science 302 (5644):445-9, 2003).

In one approach, therapeutic efficacy is assessed by measuring an increase or reduction in cell death, including apoptosis, depending on the disease. Apoptotic cells are characterized by morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, CA), the ApoBrdU DNA Fragmentation Assay (BIOVISION, Mountain View, CA), and the Quick Apoptotic DNA Ladder Detection Kit (BIOVISION, Mountain View, CA).

### Kits

The invention provides kits for the treatment or prevention of a neurological disease. In one embodiment, the kit includes a composition comprising a nanoparticle of the invention, e.g., a nanoparticle having a capture molecule (e.g., a ligand) that specifically binds a cell surface marker of a microglia, and a therapeutic and/or diagnostic agent. In some embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired an agent of the invention is provided together with instructions for administering the agent to a subject having or at risk of developing a disease. In certain embodiments, the disease is a neurological disease or disorder of the central nervous system, including a disease or disorder associated with neuroinflammation. In other embodiments, the disease or disorder is cancer or neoplasia. The instructions will generally include information about the use of the composition for the treatment or prevention of the disease or disorder. In other embodiments, the instructions include at least one of the following: description of the therapeutic agent; dosage schedule and administration for treatment or prevention of a neurological disease or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, therapeutic and/or diagnostic methods of the invention, and are not intended to limit the scope of what the inventors regard as their invention.

### Examples

### Example 1. Synthesis and Characterization of a New Class of Nanoparticles (NPs), Suitable for Drug and Gene Delivery and with Features Optimized for Surface Functionalization and In Vivo Administration

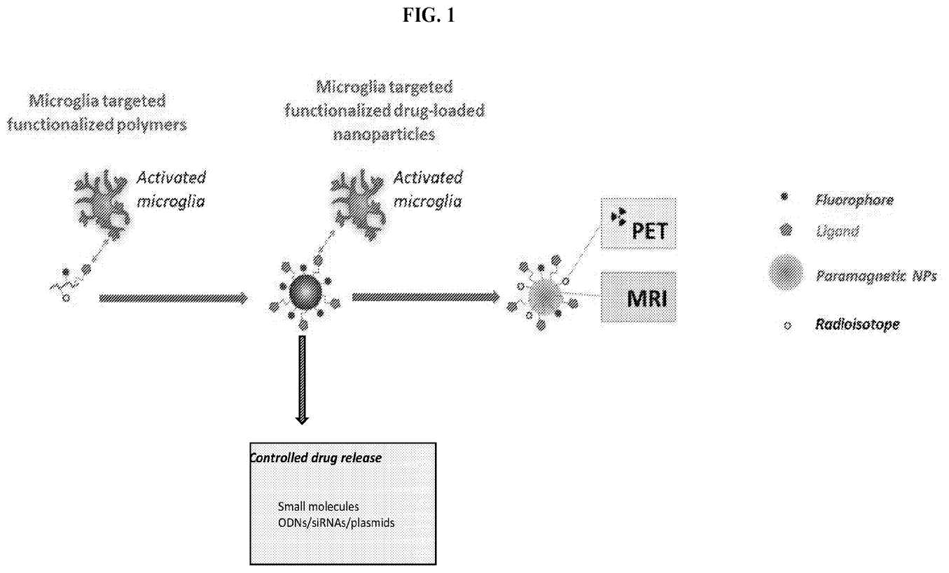

A novel class of nanoparticles (NPs) were obtained, optimized for better biodistribution in vivo and for functionalization with: i) TSPO-selective ligands; ii) moieties allowing loading and controlled release of small molecules and/or oligodeoxynucleotides in vivo; and iii) functional groups allowing MRI/PET traceability (FIG. 1).

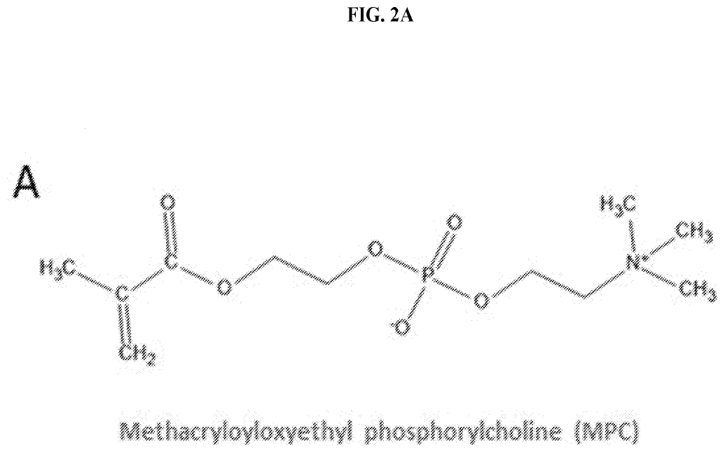

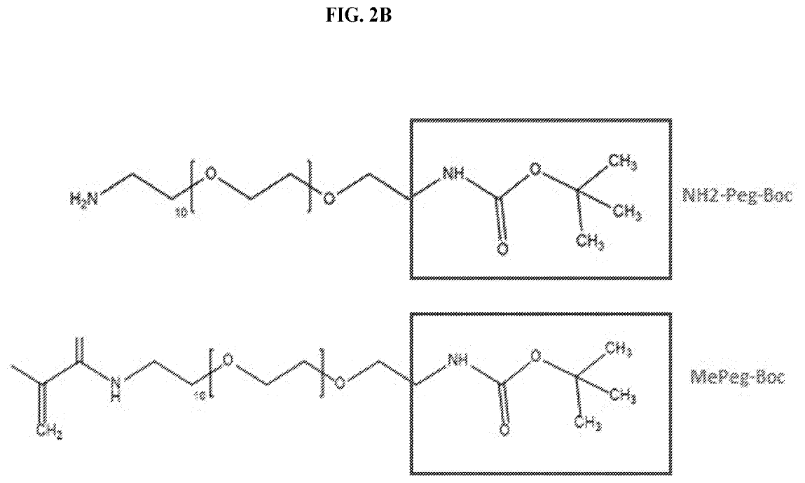

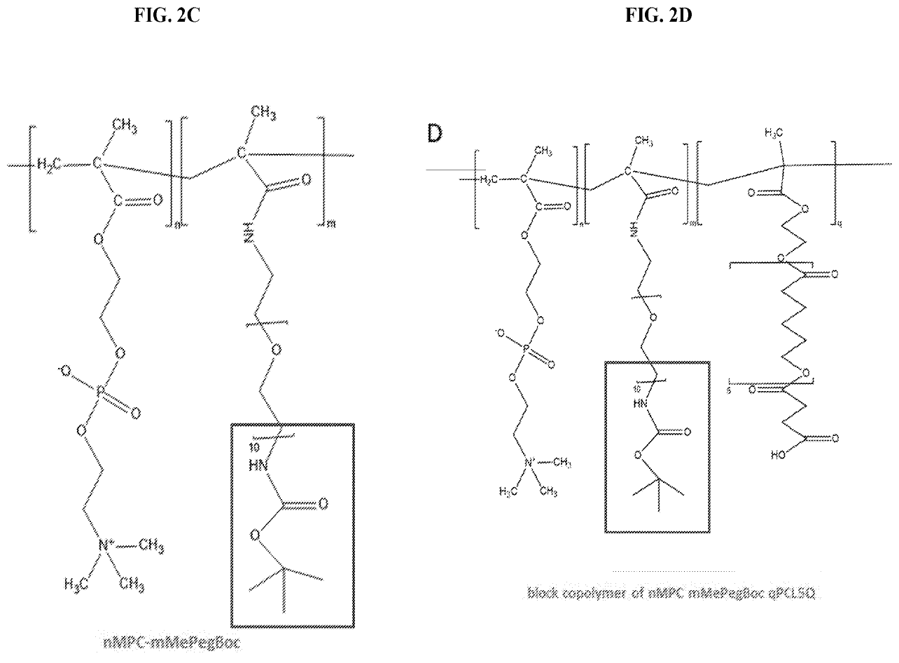

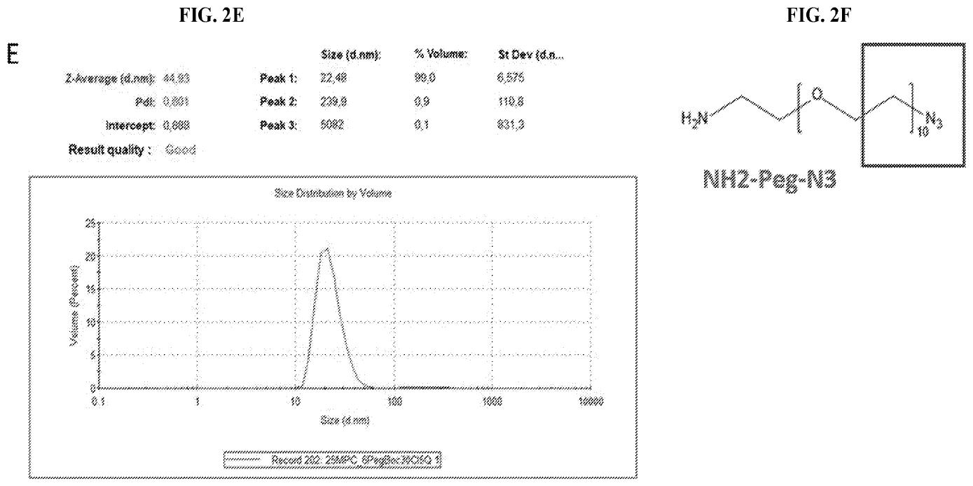

2-Methacryloyloxyethyl phosphorylcholine (MPC) was utilized as a starting monomer (FIG. 2A). MPC contains a phosphorylcholine group mimicking the phospholipid polar groups contained within cell membranes, resulting in high biocompatibility. In addition, a PEGylated monomer was specifically synthesized to covalently bind the precursors of translocator protein (TSPO) ligands already validated in the clinical setting, i.e. PK11195 and PBR28. In particular, the NH2-PEG-Boc (FIG. 2B) or, alternatively the NH2-PEG-N3 (FIG. 2F), precursor was first provided with a methacrylamide group (Me-PEG-Boc, FIG. 2B, or Me-PEG-N3, respectively) and subsequently polymerized together with MPC via RAFT (Reversible addition-fragmentation chain-transfer) polymerization in a ratio of 25:3 and 25:6. The conversion was evaluated by NMR, and the polymer was purified by dialysis and then precipitated in acetone (nMPC-mMePegBoc, FIG. 2C). This polymer was then used like a macro-RAFT agent to produce a block copolymer for the polymerization of a biodegradable hydrophobic monomer (HEMA-polycaprolactone5) modified with a COOH ending group. 25MPC-3MePeg-Boc and 25MPC-6MePegBoc were polymerized through RAFT polymerization with HemaCl5Q and Hema-Rhodamine (FIG. 2D). The resulting block copolymer was nanoprecipitated to produce rhodaminated nanoparticles. These NPs (hereafter “NP-MPC”) are about 45 nm in diameter and very homogeneous, with narrow polydispersion (FIG. 2E).

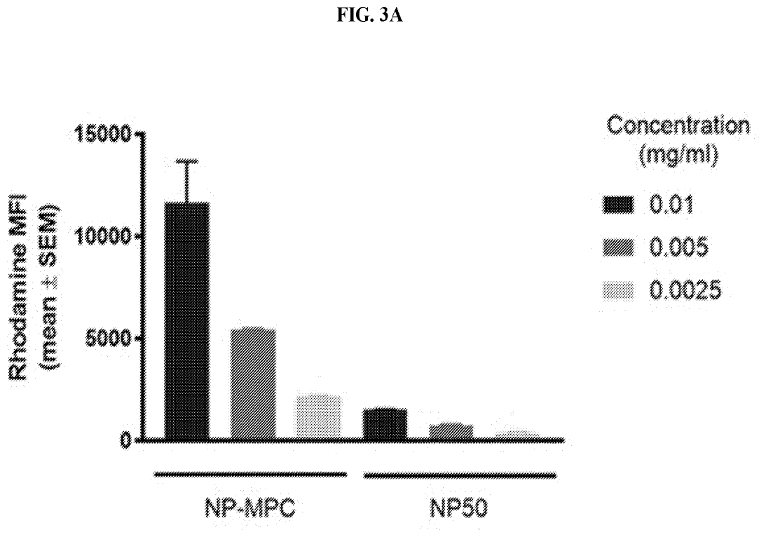

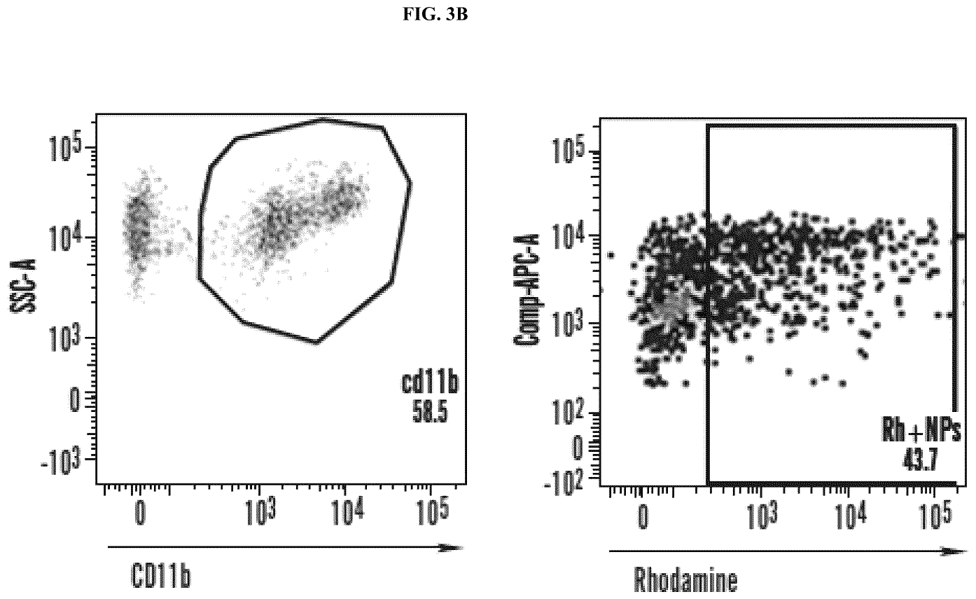

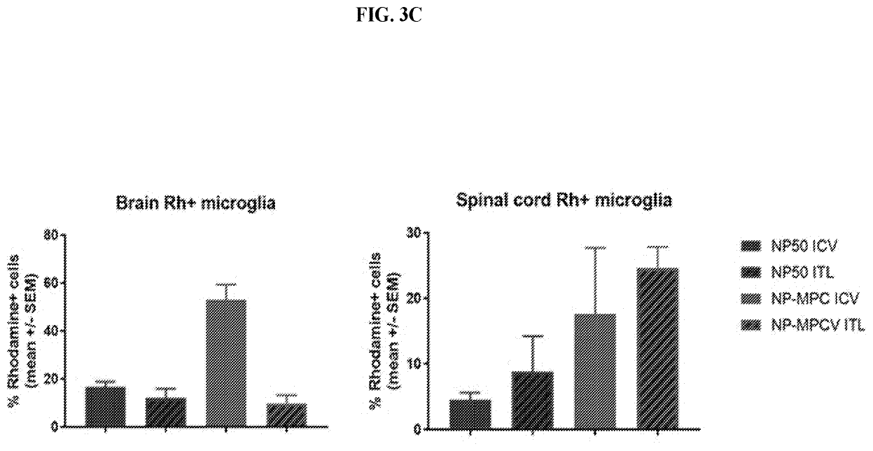

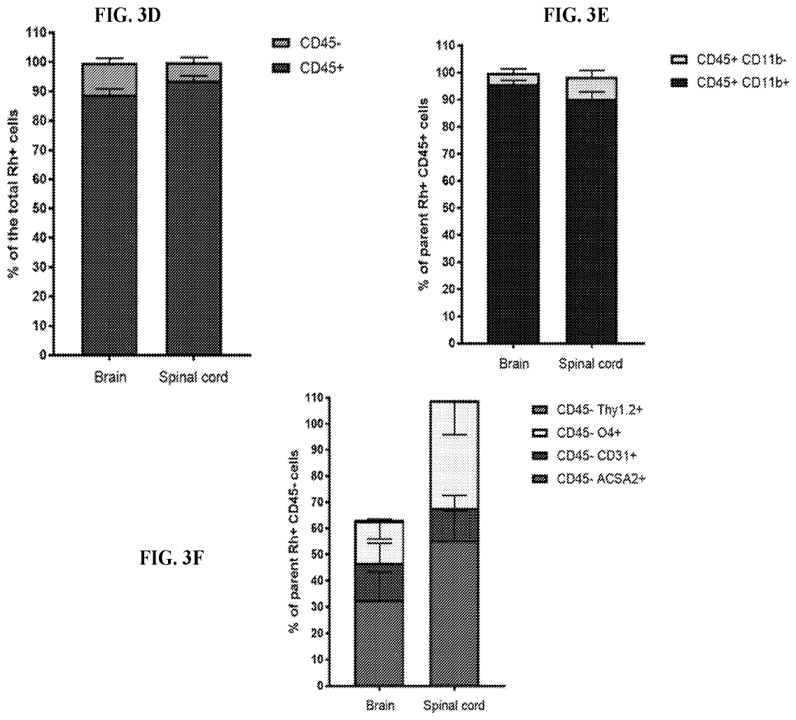

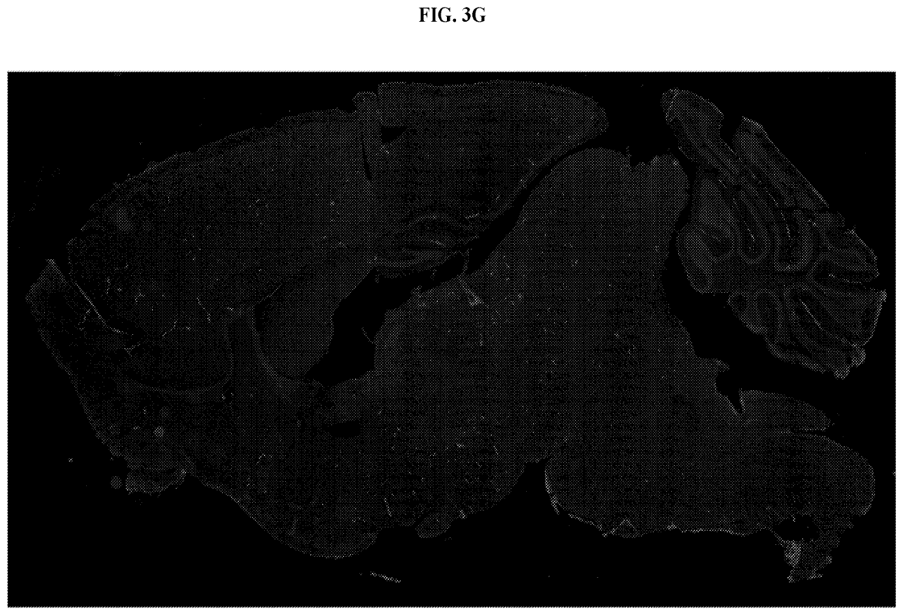

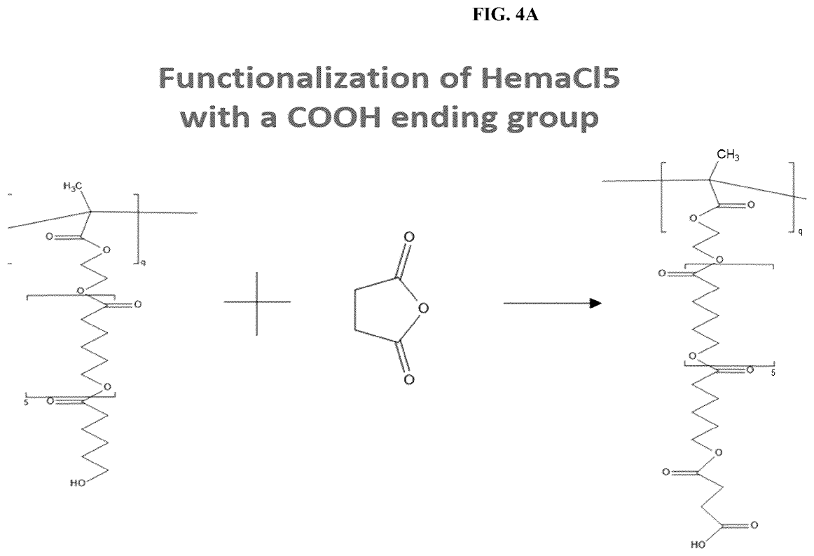

The uptake of NP-MPC was assessed in vitro in BV2 cell lines. NPs were added to cell culture medium at different concentrations for 24 hours; then cells were extensively washed and collected by trypsinization to analyze by flow cytometry the percentage of rhodamine⁺ cells. A batch of rhodaminated NPs (hereafter called NP50 because their size is 50 nm) was also tested as reference. NP50 is non-functionalized and validated for microglia uptake. As shown in FIG. 3A, the uptake of NP-MPC was more efficient than NP50 at all tested concentrations. Interestingly, the in vitro results were recapitulated in the in vivo setting (FIGS. 3B-3C). Briefly, 5 μl of NPs (either NP-MPC or NP50 at 0.2% w/v in PBS), were administered to 12-15 week-old healthy mice in the cerebrospinal fluid (either intracerebroventricularly (ICV), i.e., in the cerebral lateral ventricles, or intrathecally into the lumbar region (ITL), i.e., in the subarachnoid space at lumbar level). All mice received an intraperitoneal injection of Mannitol 23% 1 hours after administration of NPs to obtain transient blood-brain barrier (BBB) disruption. Mice were then sacrificed 3 days after administration of NPs to analyze biodistribution of NPs by flow cytometry.

A striking increase in the percentage of rhodamine⁺ microglia cells (identified by the CD45⁺/CD11b⁺ gating, FIG. 3B) was observed for NP-MPC in the brain (up to about 50% upon ICV injection) and in the spinal cord (up to almost 25% through both routes of administration) (FIG. 3C). The majority of administered NPs is internalized in CD45⁺ cells (FIG. 3D), of which CD45⁺/CD11b⁺ microglia represents more than 90% (FIG. 3E). A few NPs (less than 10% of total) can be internalized in CD45⁻ cells, mainly astrocytes and oligodendrocytes (FIG. 3F). As shown in FIG. 3G, ITL administration of NPs allows to obtain widespread distribution of NPs throughout the brain parenchyma.

Overall these data support NP-MPC as a suitable NP platform, with dimensions and surface features allowing widespread distribution in the brain and spinal cord, and uptake by microglia cells.

### Example 2. Traceability of Nanoparticles (NPs) by Magnetic Resonance Imaging (MRI)

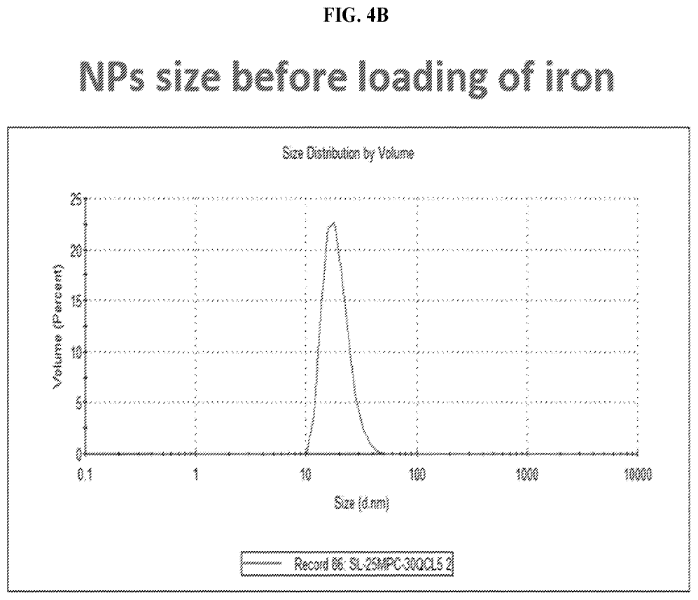

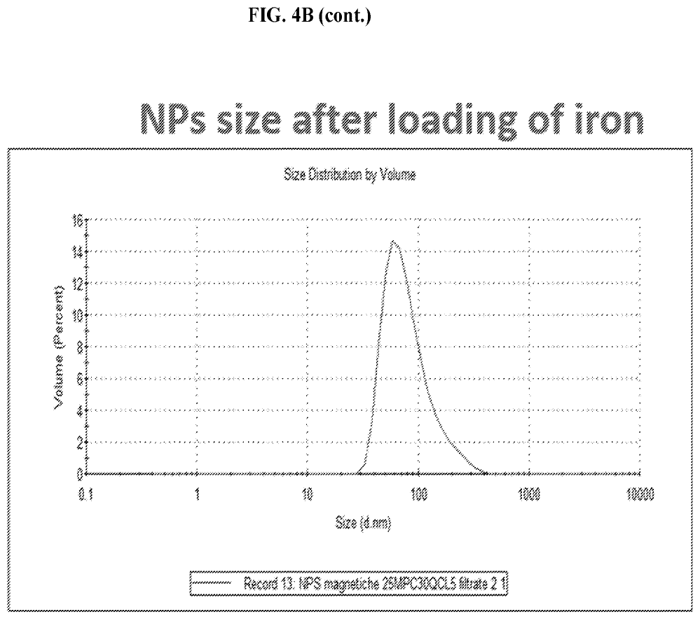

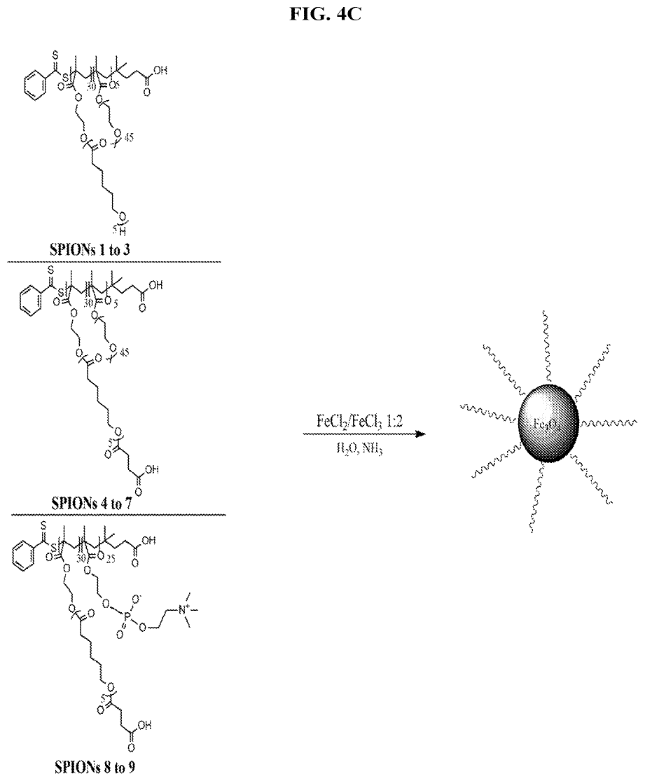

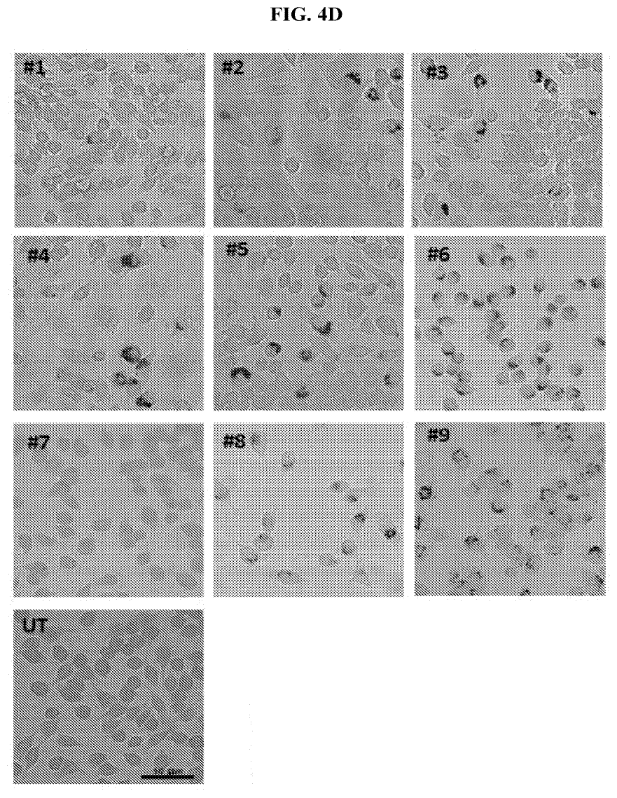

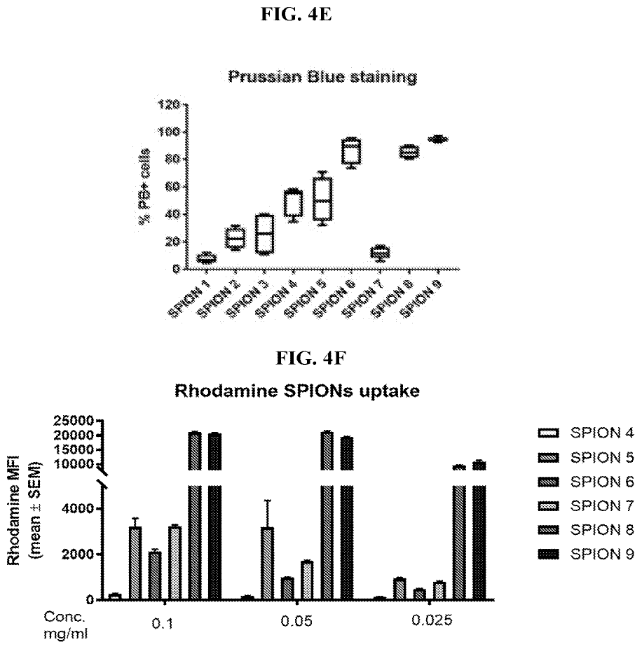

MPC was polymerized through RAFT polymerization. This polymer was then used like a macro-RAFT agent and chain extended with a biodegradable hydrophobic monomer (HEMA-polycaprolactone5) modified with a COOH ending group (FIG. 4A) to obtain a block copolymer suitable for stabilizing superparamagnetic nanoparticles in aqueous environment. 25MPC block-copolymer was polymerized through RAFT polymerization with HemaCl5Q and HEMA-Rhodamine, purified, and precipitated in diethyl ether; then it was solubilized in 3 ml of DMSO and nanoprecipitated in 20 ml of water under strong stirring. FeCl₂ (43 mg) and FeCl₃ (117 mg) were solubilized in 5 mL of distilled water, stirred for 30 minutes, and then combined with the NPs in a round bottom flask under strong stirring after degassing for 30 minutes. NH₃ 28% (1.2 ml) was then added dropwise and left under strong stirring for 30 minutes. The resulting magnetic nanoparticles were then neutralized with HCl 0.1 M, dialyzed, filtered (0.4 μm) and analyzed through dynamic light scattering (FIG. 4B).

Different batches were produced (Table 1) by using different molar ratios of iron/polymer or by adopting different formulation chemistries. SPION 1 through 7 are made by employing a PCL-based polymer platform obtained from the RAFT polymerization of PEGMA2000 for the hydrophilic block and HEMA-CL5 for the hydrophobic one. Two different strategies were adopted to coat the SPIONs with this polymer.

### 1. SPIONs 1, 2 and 3

SPIONs have been synthesized following the co-precipitation method developed by Massart. More specifically, FeCl₃ and FeCl₂ have been used as the precursors and dissolved in water in a 2:1 mole ratio. Oleic acid was used as the stabilizer in an amount as to obtain a 50% magnetite content over the total nanoparticle weight, dissolved in acetone, and added to the precursor solution. Magnetite precipitation was allowed by the addition of a 28-30% w/w ammonium hydroxide aqueous solution at 80° C. The SPION suspension was stirred at 80° C. for 1 hour in order to let the precipitation and the binding of the oleic acid to occur. Then a 10-fold excess of acetone (with respect to water) was added to precipitate the magnetite, which was collected with a magnet. The SPIONs were washed three times with acetone and then dried at room temperature overnight. The nanoparticles were resuspended in tetrahydrofuran under magnetic stirring. Different percentages of the polymer were added to this organic solution and further precipitated in water under probe sonication for 30 minutes. The tetrahydrofuran was finally removed via dialysis.

### 2. SPIONs 4, 5, 6 and 7

For SPIONs 4 to 7, polymer was functionalized with carboxyl end groups in order to let its directly use as the stabilizer during the SPION synthesis. The necessary amount of polymer to obtain a 20% magnetite content over the total weight of the nanoparticles was dissolved in DMSO with a concentration of 10% w/w. The organic phase was added dropwise to the aqueous solution of FeCl₃ and FeCl₂ (with the same 2:1 mole ratio as described previously). After stirring for 30 minutes, the system was heated to 80° C. and ammonium hydroxide was added under vigorous agitation to obtain the magnetite precipitation. The SPIONs were collected with a magnet after the addition of a 10-fold excess of acetone and dialyzed against water for three days.

### 3. SPIONs 8 and 9

SPIONs 8 and 9 are based on the NP-MPC platform. MPC was polymerized through RAFT polymerization. This polymer was then used like a macro-raft agent to produce a block copolymer for the polymerization of a biodegradable hydrophobic monomer (HEMA-polycaprolactone5) modified with a COOH ending group (FIG. 4A). 25MPC block-copolymer was polymerized through RAFT polymerization with the HemaCl5Q and HEMA-Rhodamine, purified, and then precipitated in diethyl ether. It was then solubilized in DMSO and nanoprecipitated in water under strong stirring. FeCl₂ and FeCl₃ were solubilized in distilled water and left under stirring for 30 minutes and then combined with the NPs in a round bottom flask under strong stirring after degassing for 30 minutes. 1.2 ml of NH₃ 28% was added dropwise and underwent strong stirring for 30 minutes. The resulting magnetic nanoparticles were then neutralized with HCl 0.1 M, dialized, filtered (0.4 μm) and analyzed through dynamic light scattering (FIG. 4A). For SPION 9, a new method of production was adopted, consisting of a one-pot procedure. Briefly, the poly(MPC) was synthesized as described previously. After 24 hours of reaction at 65° C., the monomer conversion was evaluated at higher than 99%, so the chain extension with the HEMA-CL5Q could be carried out without intermediate purification steps. In particular, 0.94 g of HEMA-CL5Q (target DP=30), 2 mg of HEMA-Rhodamine and 4 mg of ACVA (macro CTA/ACVA=3) were dissolved in 1 g of ethanol and added to the solution containing the macro CTA. The reaction was left to occur for an additional 24 hours at 65° C. during which the simultaneous chain-extension of the macro CTA with the hydrophobic HEMA-CL5Q and the block copolymer self-assembly occur. Therefore, the traditional post-polymerization processes to obtain NPs from amphiphilic block copolymers, such as nanoprecipitation, emulsion-evaporation (which generally require dilute conditions) were avoided in this case.

FIG. 4C outlines the main features of the polymers used to prepare SPIONs 1-3, SPIONs 4-7 and SPIONs 8-9.

TABLE 1
Features of the different paramagnetic nanoparticle batches
tested (“PDI” denotes polydispersity index).

| | Diameter | | Loaded | Conc. Iron | |
|---|---|---|---|---|---|
| Batch ID | (nm) | PDI | compound | (% w/wNPs) | Fluorescence |
| SPION 1 | 145 | 0.28 | Fe3O4 | 2.5 | none |
| SPION 2 | 99 | 0.26 | Fe3O4 | 5 | none |
| SPION 3 | 92 | 0.32 | Fe3O4 | 10 | none |
| SPION 4 | 111 | 0.3 | Fe3O4 | 20 | Rhodamine |
| SPION 5 | 191 | 0.21 | Fe3O4 | 20 | Rhodamine |
| SPION 6 | 105 | 0.28 | Fe3O4 | 15 | Rhodamine |
| SPION 7 | 79 | 0.31 | Fe3O4 | 15 | Rhodamine |
| SPION 8 | 101 | 0.24 | Fe3O4 | 20 | Rhodamine |
| SPION 9 | 121 | 0.31 | Fe3O4 | 20 | Rhodamine |

The efficiency of uptake of the different NPs batches was assessed on BV2 cell lines. Cells were plated on glass coverslips (30,000 cells/well in 24-well plates) and then incubated with different concentration of SPIONs (in the range 0.2-0.05 mg/ml polymer) for 24 hours. The different batches were matched for the amount of total polymer added in culture, irrespectively of the total amount of iron content. After the incubation, the cells were fixed with 4% buffered paraformaldehyde for 20 min at room temperature (RT). Then cells were permeabilized for 15 min at RT with 0.1% Triton in PBS. Total iron content was visualized by Prussian Blue staining (consisting in the incubation for 2 hours at RT with a solution made of 2.5% hydrochloric acid, 2.5% potassium ferrocyanide). Cells were finally counterstained with Nuclear Fast Red and mounted on microscopy slides.

For SPION 4 through 9 (rhodaminated NPs), a fraction of the cells was also collected after trypsinization and then analyzed by flow cytometry to evaluate the total percentage of rhodamine positive cells, as measurement of the overall NPs uptake. As shown in FIG. 4D, iron was detected in all the tested conditions. However, the best results in terms of number of cells showing detectable amounts of iron were obtained with SPION 6, 8, and 9 (see FIG. 4E). For SPION 4 through 9, the efficiency of uptake was verified by flow cytometry, reporting almost 100% of cells that were rhodamine⁺ at all tested SPION concentrations (0.1, 0.05, and 0.025 mg/ml). Measurement of the mean fluorescence intensity of rhodamine signal highlighted a higher efficiency of uptake for SPION 8 and 9 (FIG. 4F), which was in line with the striking efficiency of uptake previously reported for NP-MPC.

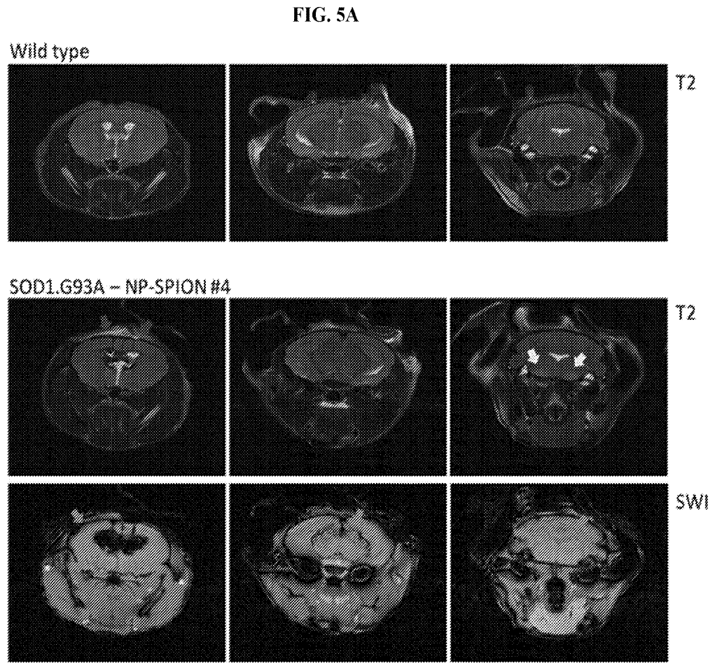

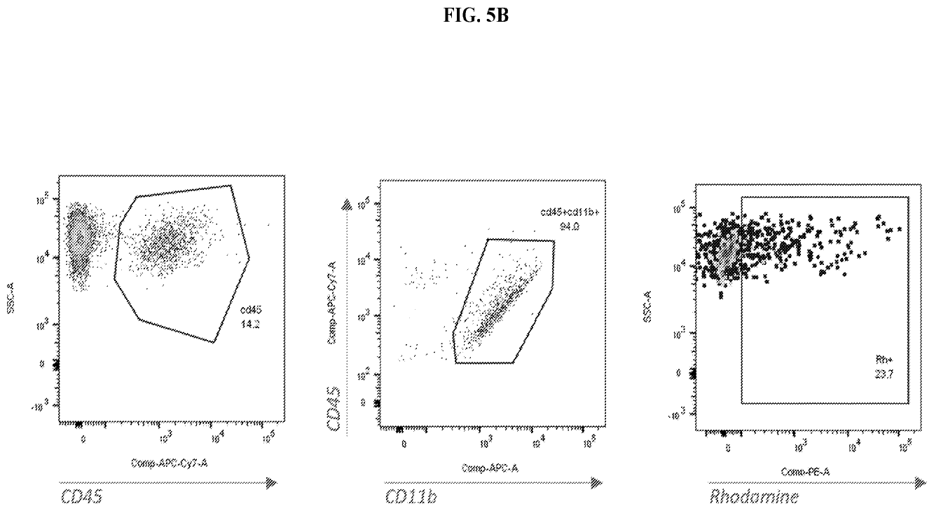

As proof of concept for detecting NPs biodistribution in vivo by MRI, SPION 4 nanoparticles were administered (5 μl total volume) ICV to symptomatic SOD1.G93A or to wild type mice. Three (3) days after administration, mice underwent MRI (T2 anatomical axial and sagittal view, T2 and T2* maps and susceptibility weight imaging, SWI maps). As shown in FIG. 5A (arrows in SWI panels), a massive NP signal was detected in the lateral ventricles and strikingly, positively widespread throughout the cerebral cortex, rostral to caudal. As expected for symptomatic SOD1.G93A mice, T2 images highlighted hyper-intensive signal in the trigeminal (not shown), hypoglossal (not shown) and facial nucleus (yellow arrows in FIG. 5A, arrows in right T2 panel). Cytofluorimetric analysis of rhodaminated NP-SPION biodistribution confirmed uptake by CNS CD45⁺/CD11b⁺ microglia cells (FIG. 5B).

### Example 3. Traceability of Nanoparticles (NPs) by Positron Emission Tomography (PET)

As described above, NPs-MPC have been designed as an innovative platform allowing multiple functionalization. The goal is to exploit these NPs not only for MRI but also for PET imaging. The advantage of PET over MRI is that this technique is highly sensitive and allows precise quantification of the radionuclide in vivo. Thus, PET traceable NPs will be instrumental for monitoring and quantifying neuroinflammation in vivo. Cytofluorimetric analyses and in vivo MRI data showed many NPs still detectable in the CNS at 3 days after injection. The levels of NPs are stable up to about 7 days post-injection. Afterwards they are gradually degraded and washed out of the tissue within about 30 days. Thus, for PET, a radioisotope was chosen with a half-life fitting the dynamics of NP biodistribution in vivo. Zirconium⁸⁹ (⁸⁹Zr), with a half-life of 78.4 hours (about 3 days) was identified as the best isotope suited for these studies. The conjugation of Deferoxamine (a chelator) to NP-MPCs was identified as an important step necessary to allow efficient loading of Zirconium⁸⁹ on the NPs. The reaction can be performed in aqueous solution; thus, the loading of the radionuclide on the surface of the preformed NPs is envisaged.

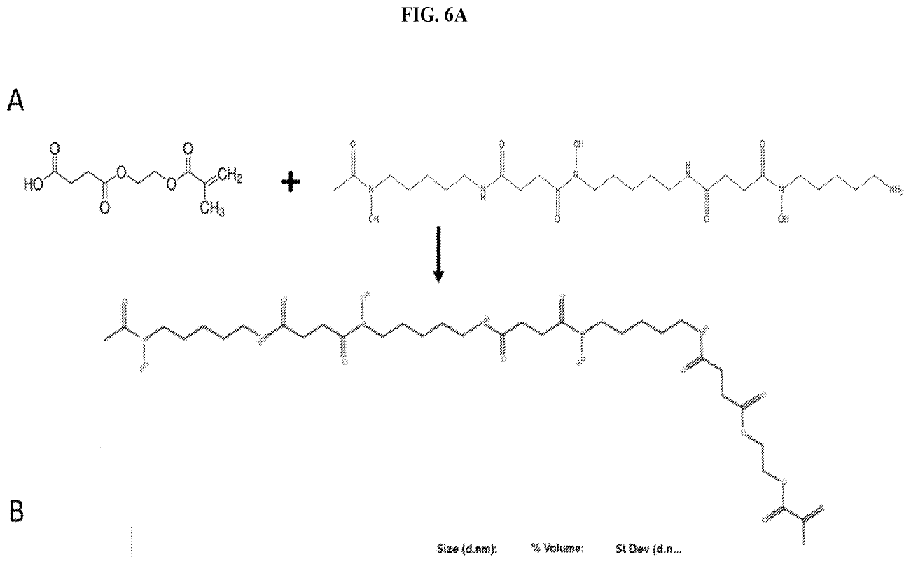

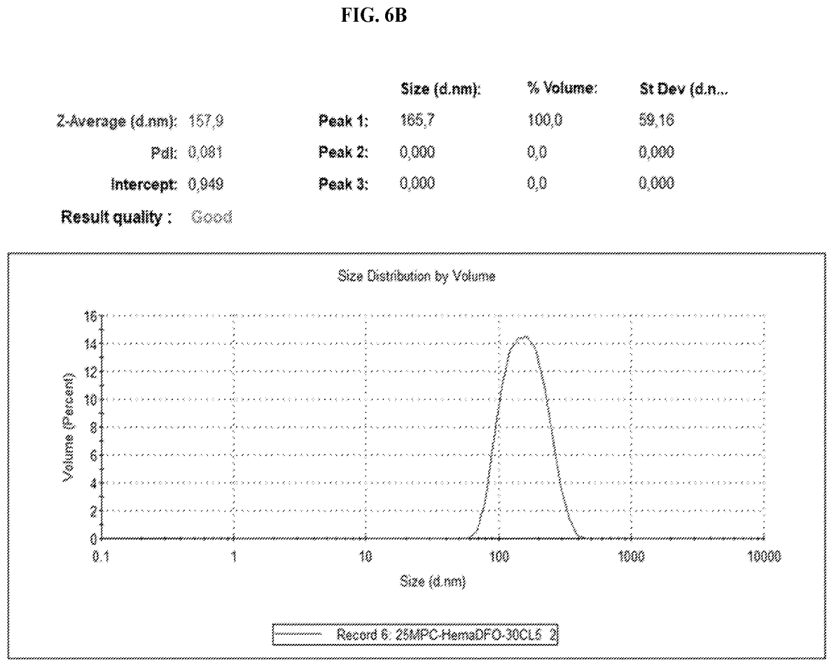

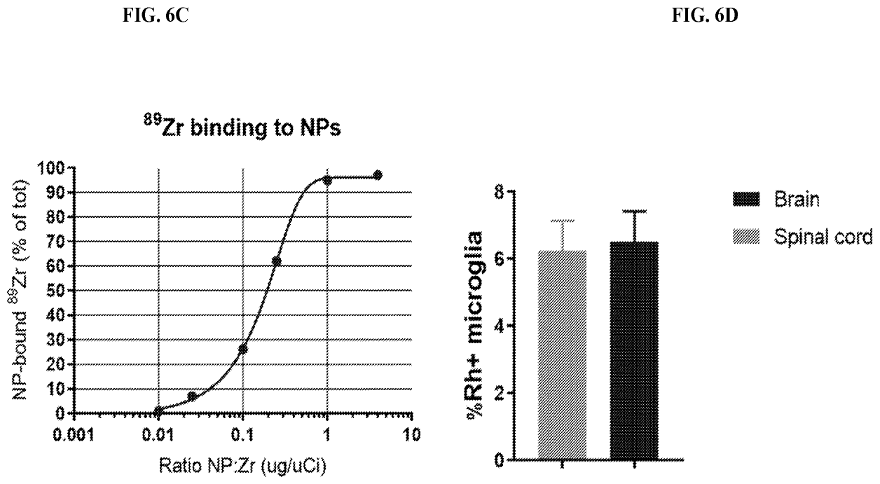

A strategy to conjugate covalently deferoxamine on the surface of NP-MPCs is depicted (shown in FIG. 6A). MPC was polymerized through RAFT polymerization. Mono-2-(Methacryloyloxy)ethyl succinate was functionalized with deferoxamine (DFO) mesylate though steglich esterification. The so called Hema-DFO was then polymerized with 25MPC to create a hydrophilic macro-agent functionalized with DFO. 25MPC-HemaDFO was polymerized through RAFT polymerization with HemaCl5 and HemaRhodamine. The block copolymer so created was nanoprecipitated to produce nanoparticles with diameters of about 160 nm (FIG. 6B).

To determine the extent of nanoparticle (NP) DFO functionalization, the following ⁸⁹Zr binding assay was performed: Six concurrent radiolabeling reactions were run using ˜100 μCi of ⁸⁹Zr oxalate each, and varying quantities of DFO-functionalized nanoparticles (FIG. 6C). After 60 minutes, each crude reaction mixture was analyzed by radio-TLC, where unbound ⁸⁹Zr was found to migrate with the 20 mM sodium citrate solvent front while NPs remained at the baseline. Using the previously determined ⁸⁹Zr decay-corrected specific activity (Ci/μmol) and the radio-TLC data (% bound vs. unbound), calculation of the DFO:NP functionalization ratio was estimated to be ˜4.7:1. Without being bound by theory, the current DFO functionalization ratio allows for an injectable product activity to mass ratio of approximately 100 μCi per 100 μg of NPs.

As shown in FIG. 6D, NPs functionalized with DFO can also be conjugated covalently to a rhodamine dye, allowing cytofluorimetric analyses or fluorescence microscopy to detect NP internalization and biodistribution to a greater extent than PET. This further supports the suitability of the NP platform presented herein for the generation of multi-traceable compounds.

### Example 4. Non-Viral Nanoparticle-Mediated Gene Delivery: Preliminary Tests of the Loading Efficiency and Release Profile of Polymers Designed as Carriers for Gene Delivery

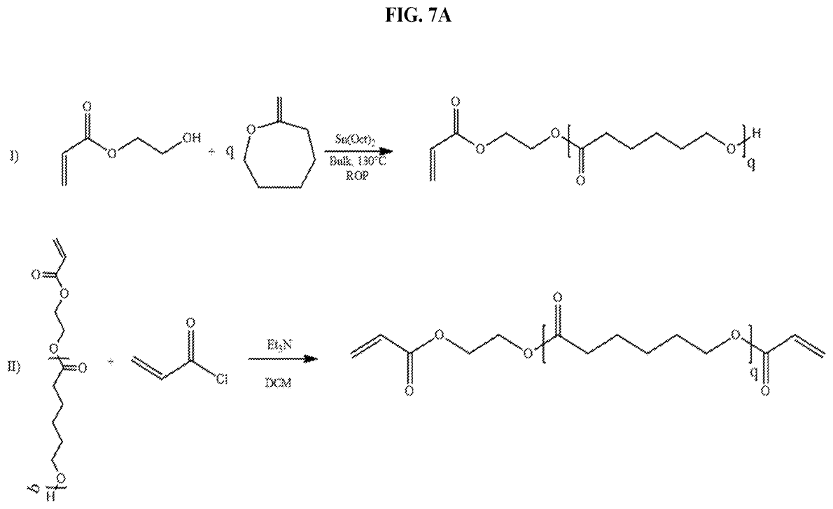

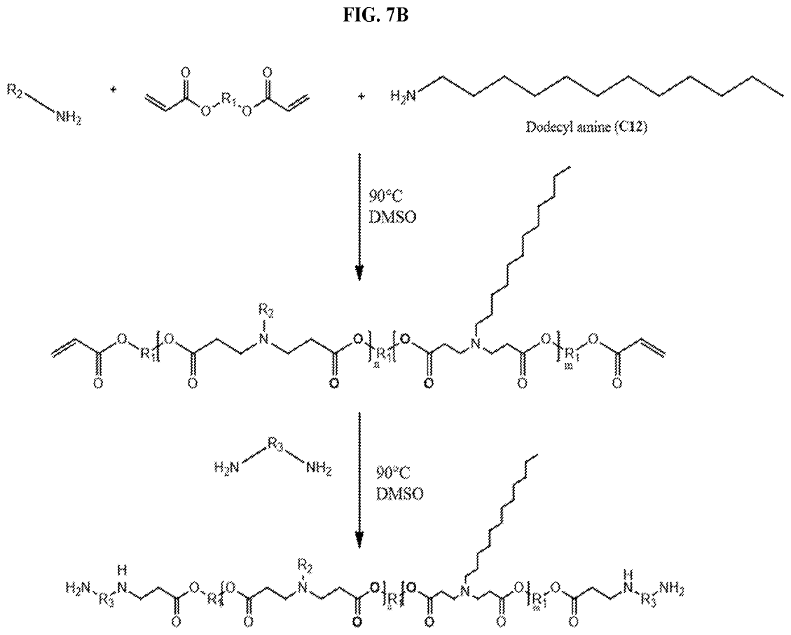

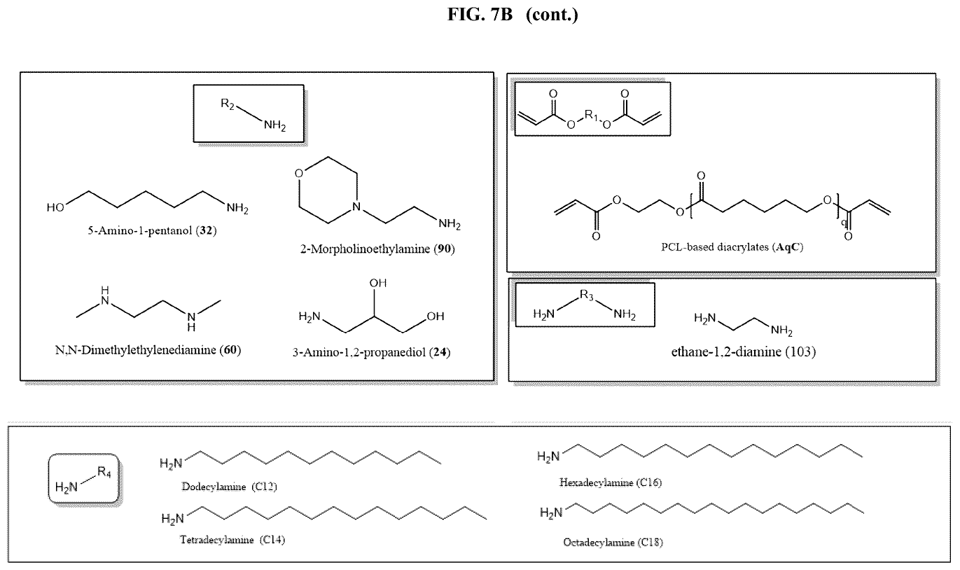

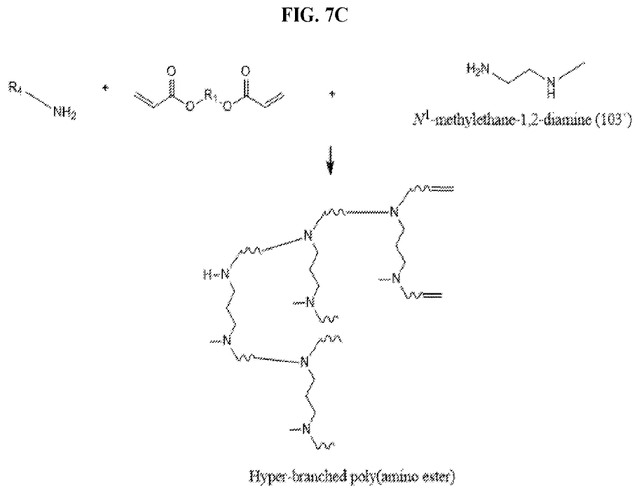

Poly (β-amino esters) (PBAEs) are a class of polymers particularly promising for gene delivery due to their facile synthesis, transfection efficiency, and degradability. PBAEs are usually produced in two steps that consist of (i) a step growth polymerization (Michael addition) between amines and a diacrylate and (ii) an end-capping of the final polymer with a diamine. The multiple ester bonds present in the polymer backbone can be degraded in the body via hydrolysis. Here, diacrylates in the synthesis of PBAE terpolymers were substituted with PCL-based ones that are expected to be more biocompatible. The PCL-based diacrylates were synthesized via a two-step procedure that consists of (i) a ring opening polymerization of caprolactone (CL) with hydroxyethylacrylate (HEA) as initiator and tin(II)-ethylhexanoate as catalyst and (ii) acylation with acryloyl chloride (FIG. 7A).

Linear PBAEs incorporating the custom diacrylate with q=3, 5, and 7 were synthesized in a two-step reaction. In the first step, as shown in FIG. 7B, an alkyl amine (dodecyl amine, C12) and different hydrophilic amines (32, 90, 60, 24) were reacted with the custom diacylate AqC at a ratio equal to 1.2:0.5:0.5 (diacrylate: hydrophilic amine:dodecyl amine) to produce a library of acrylate-terminated PBAE terpolymers. The second step is carried out without any intermediate purification and consists in another Michael addition with an excess of the diamine (103) in order to completely consume the remaining double bonds in the mixture. The linear PBAEs are named according to the following nomenclature: diacrylate/hydrophilic amine/lipophilic amine/end-capping amine. As an example, “A3C-60-C12-103” refers to the linear PBAE with a diacrylate with 3 caprolactone units (A3C), the hydrophilic amine 60, the lipophilic amine C12, and the end-capping diamine 103.

Hyperbranched PBAEs (FIG. 7C) incorporating the custom diacrylate with q=3 and 5 were synthesized in a three-step reaction. In the first step, different alkyl amines (C12-C18) were reacted with the custom diacrylates at a ratio equal to 1:2 (diacrylate:alkyl amine) for one day. Then N-Methyl-1,3-diaminopropane (103′) was poured into the vial and the reaction was left to run for 48 hours in the same conditions. As a last step, 3-diaminopropane (103) was added in order to saturate the residual vinyl bonds available. The hyperbranched PBAEs are named according to the following nomenclature: diacrylate/trifunctional amine/lipophilic amine/end-capping amine. As an example, bA3C-103′-C12-103 is the linear PBAE with the diacrylate with 3 caprolactone units (A3C), the trifunctional amine 103′, the lipophilic amine C12, and the end-capping 103.

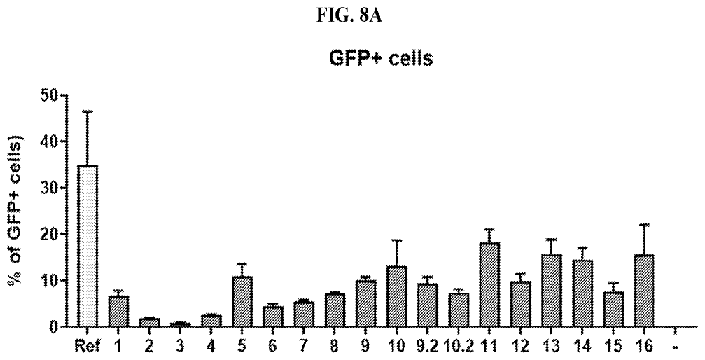

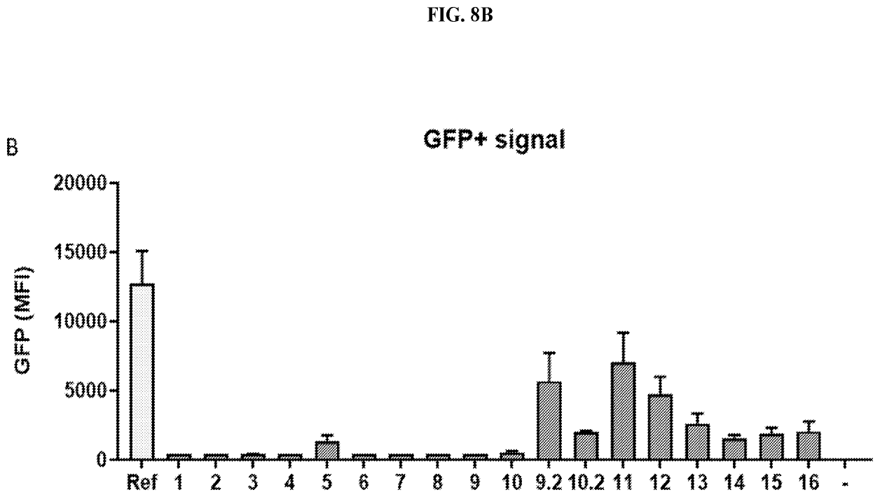

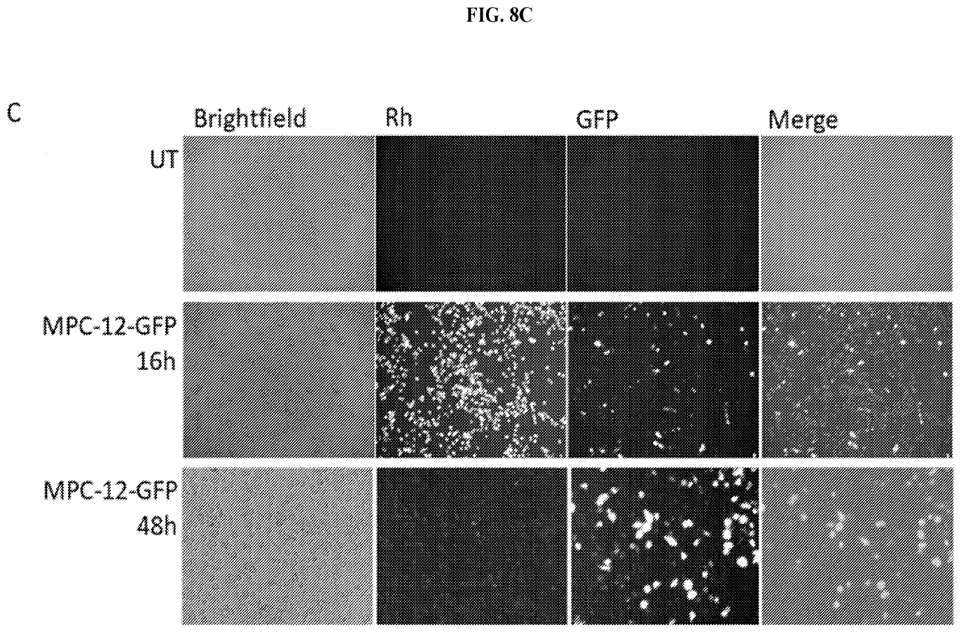

The polymer solution (PBAE in DMSO at 100 mg/ml) was directly dissolved in 25 mM NaOAc with the block-copolymer MPC (previously described) and mixed with GFP-expressing plasmid DNA to produce NPs. 18 different combinations and molar ratios of PBAE polymers were produced (Table 2). The NPs were then used to transfect BV2 cells, and the fluorescence was assessed by flow cytometry after 48 hours. Transfection of the same amount of plasmid DNA with a commercial liposomic mixture (Fugene, Promega) was used as reference (“Ref” in FIGS. 8A and 8B). As shown in FIG. 8A, GFP⁺ cells were detected with many of the 18 different formulations, with polymer #5, #10 and #11 through #16 displaying the best results in terms of transfection efficiency (measured as % of cells found positive for GFP), and with the polymers #11 through #16 displaying also the best performance when the extent of expression of the reporter gene was evaluated (FIG. 8B). These results indicate that these novel PBAEs can complex oligonucleotides (such as DNA) and that the complex can be loaded on the new polymeric MPC platform and used to form NPs able to transfer DNA into cells, as shown in FIG. 8C.

TABLE 2
PBAE terpolymers

| Sample | Name | Type |
|---|---|---|
| 1 | A3C-60-C12-103 | Linear |
| 2 | A3C-24-C12-103 | Linear |
| 3 | A3C-90-C12-103 | Linear |
| 4 | A3C-32-C12-103 | Linear |
| 5 | A5C-60-C12-103 | Linear |
| 6 | A5C-24-C12-103 | Linear |
| 7 | A5C-90-C12-103 | Linear |
| 8 | A5C-32-C12-103 | Linear |
| 9 | bA3C-103′-C12-103 | Branched |
| 10 | bA5C-103′-C12-103 | Branched |
| 9.2 | bA3C-103′-C12-103 | Branched |
| 10.2 | bA5C-103′-C12-103 | Branched |
| 11 | bA3C-103′-C14-103 | Branched |
| 12 | bA3C-103′-C16-103 | Branched |
| 13 | bA3C-103′-C18-103 | Branched |
| 14 | bA5C-103′-C14-103 | Branched |
| 15 | bA5C-103′-C16-103 | Branched |
| 16 | bA5C-103′-C18-103 | Branched |

### Example 5. Loading Efficiency and Release Profile of Small Molecules Targeted to Modulation of Microglia Reactivity

As described herein, loading and release of diapocynin, a small molecule targeted to inhibition of NADPH-oxidase 2 (NOX2) was demonstrated. NOX2 is a multi-subunit enzyme complex involved in redox stress and induction of pro-inflammatory cytokines, critically involved in the pathogenesis of several neurodegenerative diseases, including Parkinson's disease and ALS. Diapocynin, a covalent dimer of the NOX2-inhibitor apocynin, is the activated metabolite of apocynin produced by myeloperoxidase-mediated oxidation, also capable of inhibiting the expression of NOX2 mRNA and cytokines release. Without being bound by theory, administration of diapocynin removes the necessity for peroxidase involvement, which might limit diapocynin formation in vivo.

Diapocynin has a pKa of 7.4, so at physiological pH only half of the hydroxyl groups are protonated, thus causing an overall negative charge of the compound that enhances its solubility in water. For this reason, MPC-BCL3 NPs were used, composed of block copolymer complexed with a lipophilic cationic polymer (A3C32-C12-103, also called BCL3). Thus, the cationic polymer is expected to complex with diapocynin at physiological conditions to increase its loading efficiency.

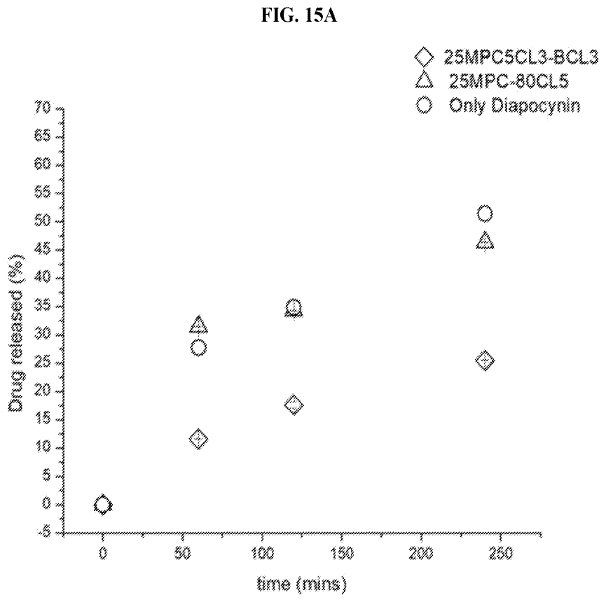

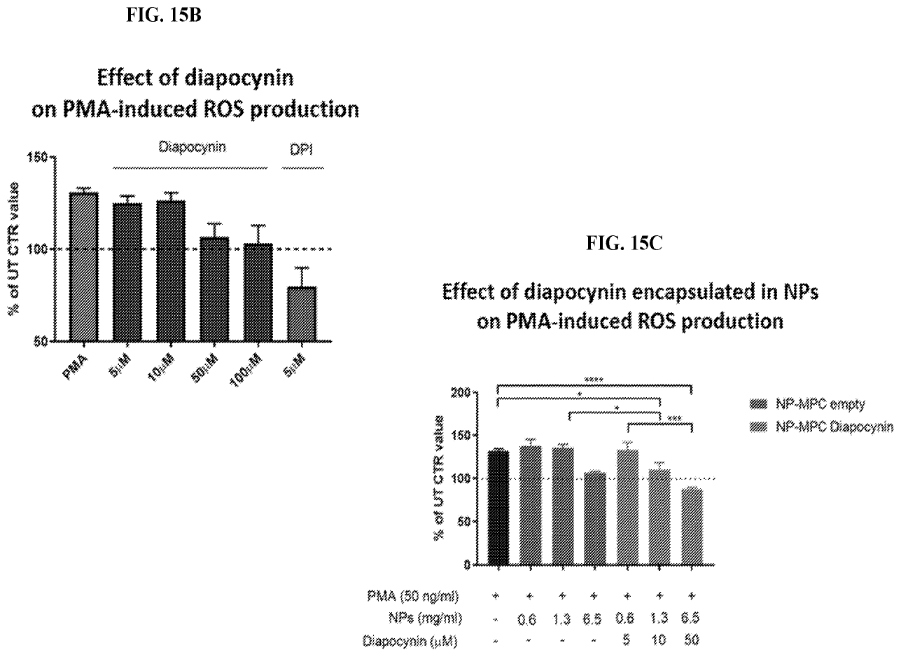

As shown in FIG. 15A, the loading efficiency, measured at t=0 and expressed as the amount of drug entrapped in the NP core compared to that loaded in the process, was 86.8%. Without being bound by theory, this indicates that the positively charged polymer allows a higher loading efficiency. Furthermore, the presence of the cationic polymer also slowed the kinetics of release of the drug compared with other NPs. In fact, after 4 hours only 25% of the drug had been released by 25MPC5CL3-BCL3 NPs compared with 50% release by the other NPs.

The WST-1 assay allows to measure the ROS produced by cell cultures. As shown in FIG. 15B, upon stimulation of BV2 microglia cell line with phorbol ester myristate acetate (PMA) 50 ng/ml for 6 hours, the WST-1 is capable of highlighting a 30-40% increase of ROS production as compared to untreated (UT) control. This effect is likely mediated by PMA-induced activation of NADPH-oxidase (NOX2), as co-incubation of cells with diphenylene iodonium (DPI), a well-known irreversible inhibitor of NOX2, is able to counteract the induction of ROS in the cultures. Diapocynin (added to the cell culture medium) is able to inhibit the PMA-induced increase of ROS at concentrations >50 M. As shown in FIG. 15C, encapsulation of diapocynin in MPC-NPs does not interfere, but rather increases, the efficacy of the drug to inhibit PMA-induced ROS release, resulting efficacious already at a nominal concentration of 10 μM.

### Example 6. Synthesis of Polymer Conjugates for Targeting the TSPO Receptor

Instrumental for proper surface functionalization of nanoparticles (NPs) is the identification of a precursor of the selected TSPO ligands that allows covalent attachment to the polymers without disrupting the binding properties of the original compounds.

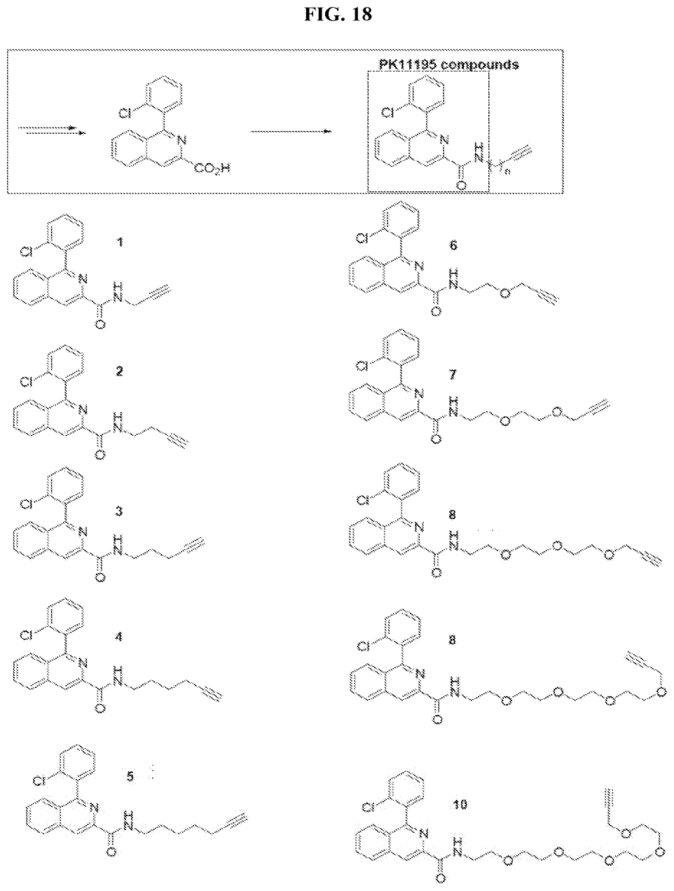

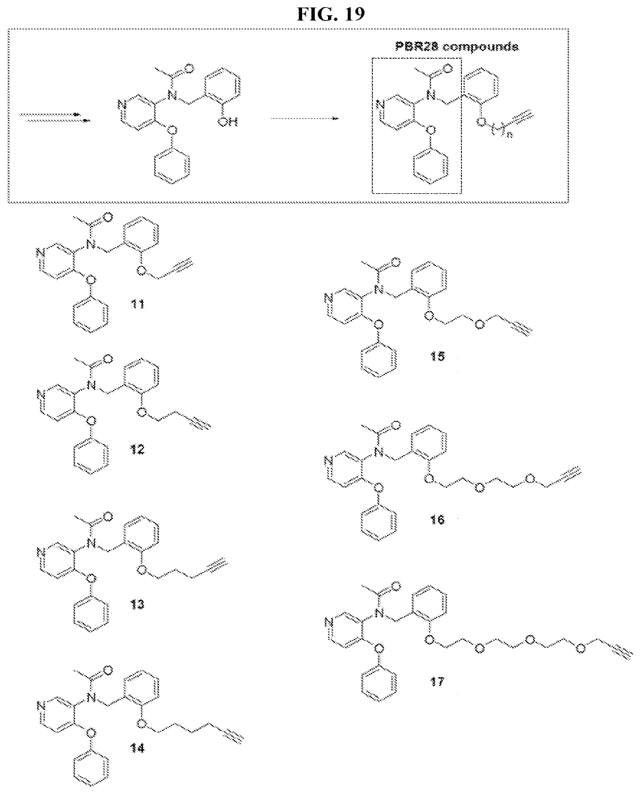

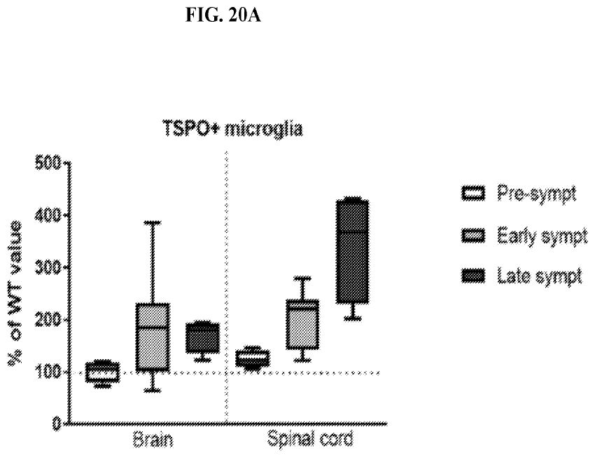

FIGS. 18 and 19 identify additional candidate TSPO-ligand precursors carrying an alkyne reactive group and suitable for NP functionalization through click-chemistry. Without being bound by theory, these compounds were designed based on the concept that the ligand should be a certain distance between the NP surface (where the conjugation of the NP with the ligand takes place) and the site of ligand-receptor interaction, to prevent possible steric hindrance affecting the binding to the receptor.

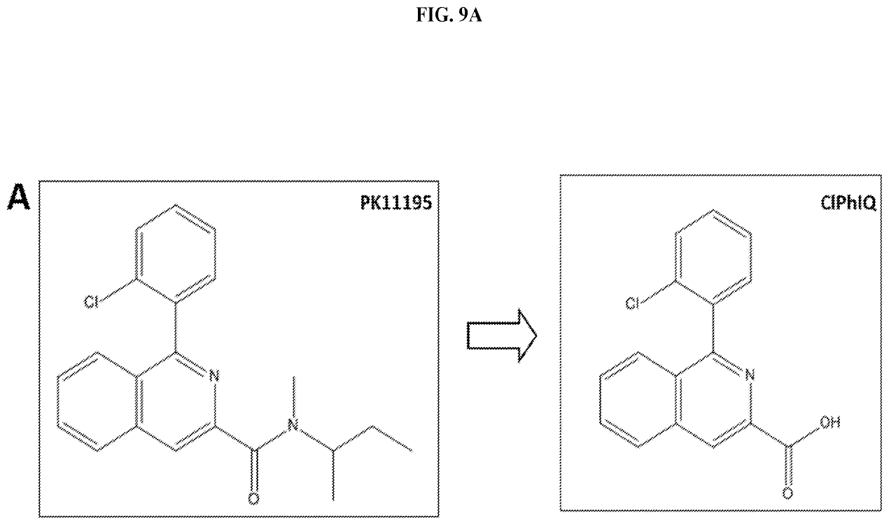

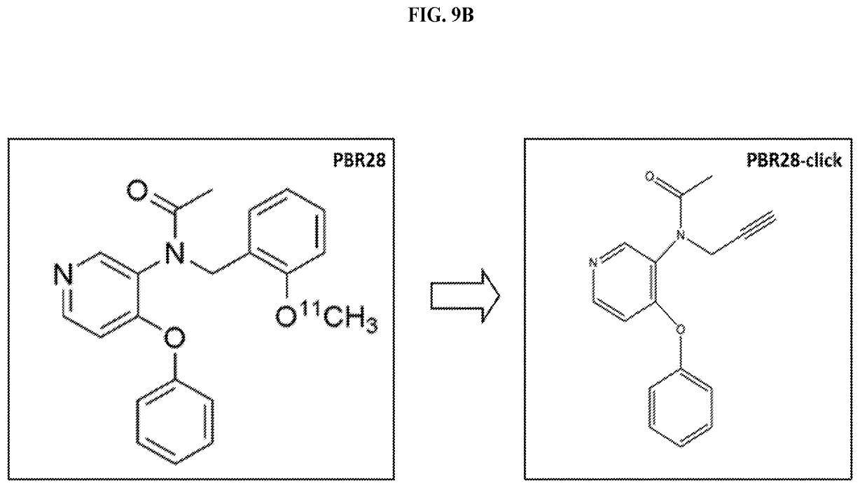

As described hereinbelow, results were obtained with two TSPO-ligand precursors as proof of concept: i) 1-(2-chlorophenyl)isoquinoline-3-carboxylic acid (ClPhIQ), a commercially available PK11195 precursor; and ii) the PBR28 precursor PBR-click (synthesized ex novo) (FIGS. 9A, 9B).

### Example 7. Synthesis and Characterization of Polymer-PK11195 Conjugates

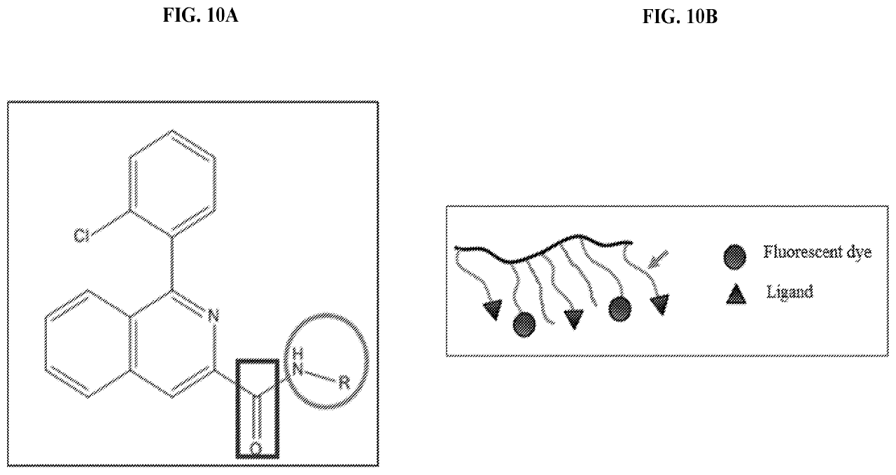

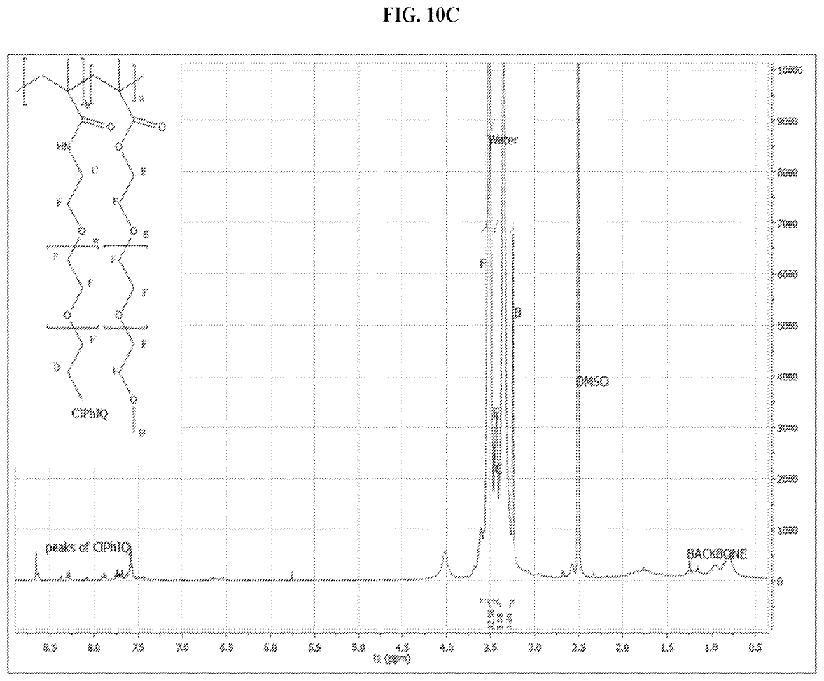

Polyethylene glycol (PEG) was utilized as the starting monomer for the synthesis of desired final branched polymers. PEG's hydrophilicity was exploited to enhance the water solubility of ligands. PEGMA-NH₂ terminated, a monomer based on PEG and having a free and accessible reactive group (—NH₂) at one end of the PEGMA, was produced via RAFT (Reversible addition-fragmentation chain-transfer) polymerization in order to obtain a high molecular weight branched polymer with terminal reactive amino-groups. Starting from the PK11195 precursor ClPhIQ, a 1-(2-chlorophenyl)isoquinoline-3-carboxylic acid derivative was generated that is bound to one of the primary amines of the synthesized polymers (FIG. 10A). This bond produces a carbonyl group (FIG. 10A) super-imposable to the carbonyl group present in the original PK11195 and known to be involved in long-range electrostatic interactions with translocator protein (TSPO) receptor.

One peculiar feature of the synthetized functionalized polymers is that they have a comb-like structure, suitable for binding at the same time the targeting moiety (1-(2-chlorophenyl)isoquinoline-3-carboxylic acid, working as a TSPO-ligand) and other tracer compounds, such as fluorescent-labeled chains (FITC or rhodamine), or radioligands for PET imaging. This allows the tracking of the compounds by fluorescent microscopy/flow cytometry or spectrofluorimetry. By controlling the stoichiometry of the reactions performed to attach the ligands or fluorescent dyes to the comb-like polymer, it is possible to obtain different nanoparticles carrying variable molar ratios of ligand/dye. FIG. 10B shows a schematic representation of the final comb-like polymer; the H-NMR characterization of the functionalized polymer is shown at FIG. 10C.

As shown in FIG. 10C, the ring hydrogens of ClPhIQ give resonance signals in the range of 7.5-8.67 ppm. These signals are also visible after dialysis purification. These results confirm that the targeting agent is properly bound with the polymer through an amide covalent bond.

The molecular weight of the polymer can be varied by changing the molar ratio between the monomers adopted and the RAFT agent. The length of the PEG side chains can be modified as well as the molar ratio of ligand and the polymer. Without being bound by theory, all these parameters can potentially influence the binding of the polymer-TSPO ligand conjugate. For this reason, materials with different features were produced (see Table 3). Ligand-dependent uptake was tested by different assays on BV2 microglia cell line, to identify the formulation allowing best selectivity of uptake.

TABLE 3
Chemical characteristics of produced polymers and results of in
vitro assays performed to validate the selectivity of uptake.

| Sample | Functionalization | Fluorescent | Mw polymer | Mw polymer | PDI | Mw PEG |
|---|---|---|---|---|---|---|
| ID | with TSPO ligand | tag | (theoretical) | (GPC) | (GPC) | side chain |
| P1 NF | NO | RhB | 13 kDa | 15.3 kDa | 1.56 | 600 Da |
| | | (0.15% w/w) | | | | |
| P3 NF | NO | FITC | 25 kDa | 19.7 kDa | 1.53 | 600 Da |
| | | (7.6% w/w) | | | | |
| P5 NF | NO | FITC | 13 kDa | 14.2 kDa | 1.62 | 600 Da |
| | | (6.1% w/w) | | | | |
| P7 NF | NO | FITC | 13 kDa | 16.1 kDa | 1.66 | 300 Da |
| | | (6.1% w/w) | | | | |
| P1 F | ClPhlQ | RhB | 13 kDa | 16.2 kDa | 1.71 | 600 Da |
| | (10.8% w/w) | (0.15% w/w) | | | | |
| P3 F | ClPhlQ | FITC | 25 kDa | 21.1 kDa | 1.65 | 600 Da |
| | (4.5% w/w) | (7.6% w/w) | | | | |
| P5 F | ClPhlQ | FITC | 13 kDa | 16.2 kDa | 1.69 | 600 Da |
| | (8.6% w/w) | (14.1% w/w) | | | | |
| P7 F | ClPhlQ | FITC | 13 kDa | 18.9 kDa | 1.73 | 300 Da |
| | (10.4% w/w) | (6.1% w/w) | | | | |

| | Assays on BV2 microglia cell line | | |
|---|---|---|---|
| | | Competition with the | |
| Sample | | uptake of free ligand | Uptake is responsive to |
| ID | Uptake | (PK11195) | LPS pre-stimulation |
| P1 NF | NO (up to the | Not tested | NO |
| P3 NF | concentration of | | |
| P5 NF | 10 μg/ml, which is the | | |
| P7 NF | maximal concentration | | |
| | tested for the corresponding | | |
| | functionalized polymers) | | |
| P1 F | Yes | Yes | NO |
| P3 F | Yes | Yes | Yes |
| P5 F | Yes | Yes | Yes |
| P7 F | Yes | Yes | NO |

(“Mw” denotes molecular weight; “GPC” denotes gel permeation chromatography, and “PDI” denotes polydispersity index.

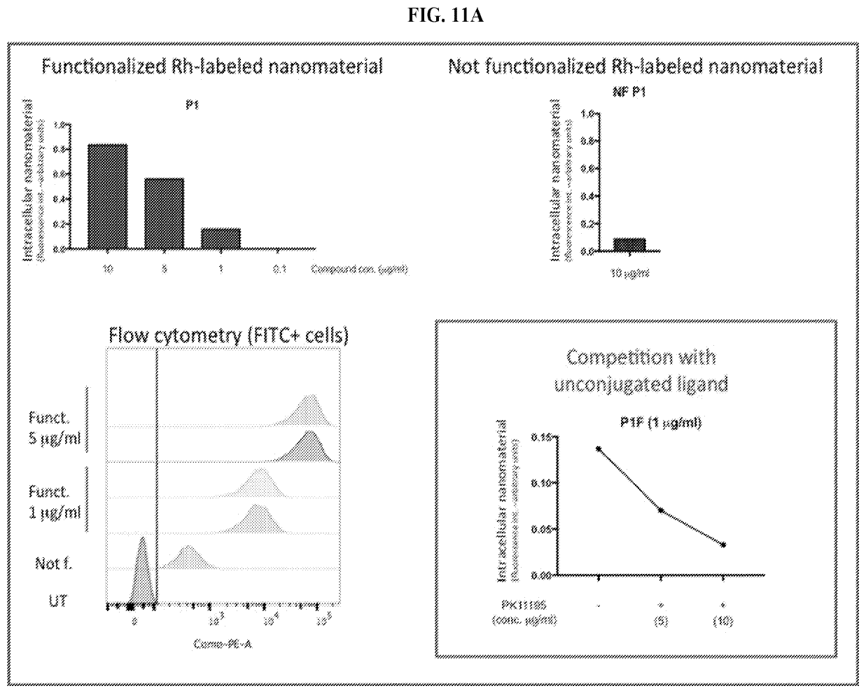

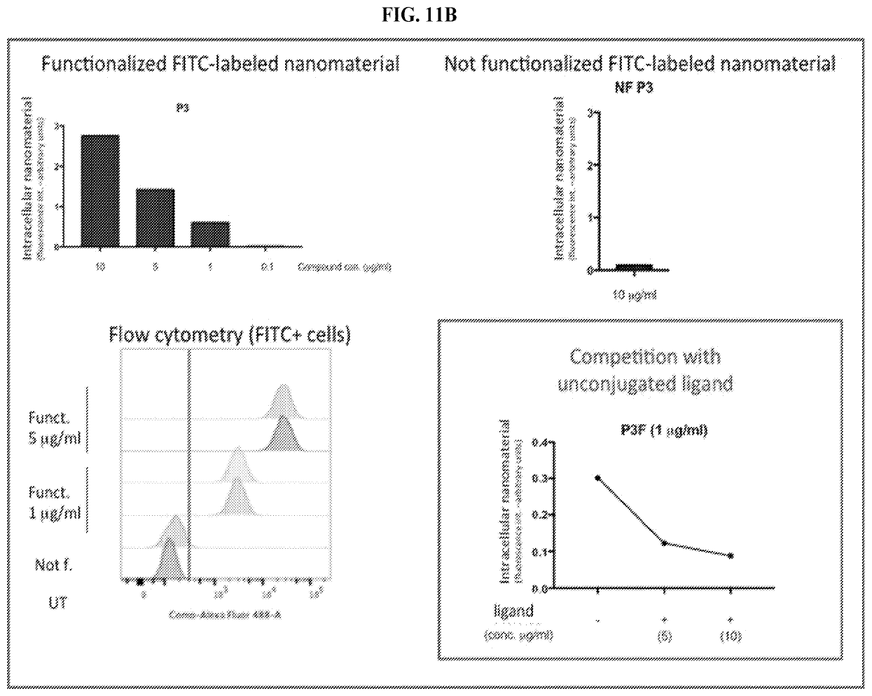

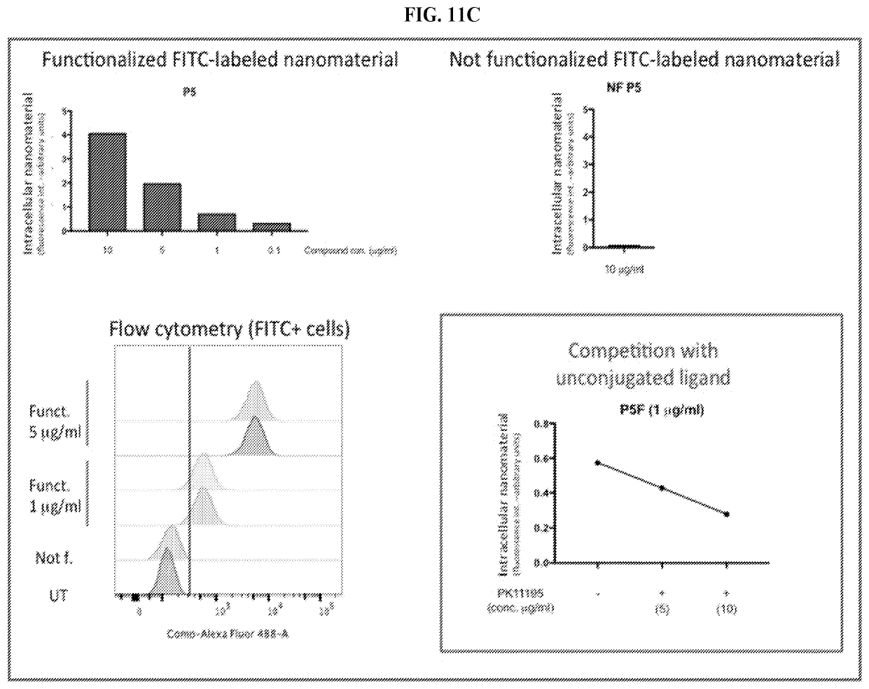

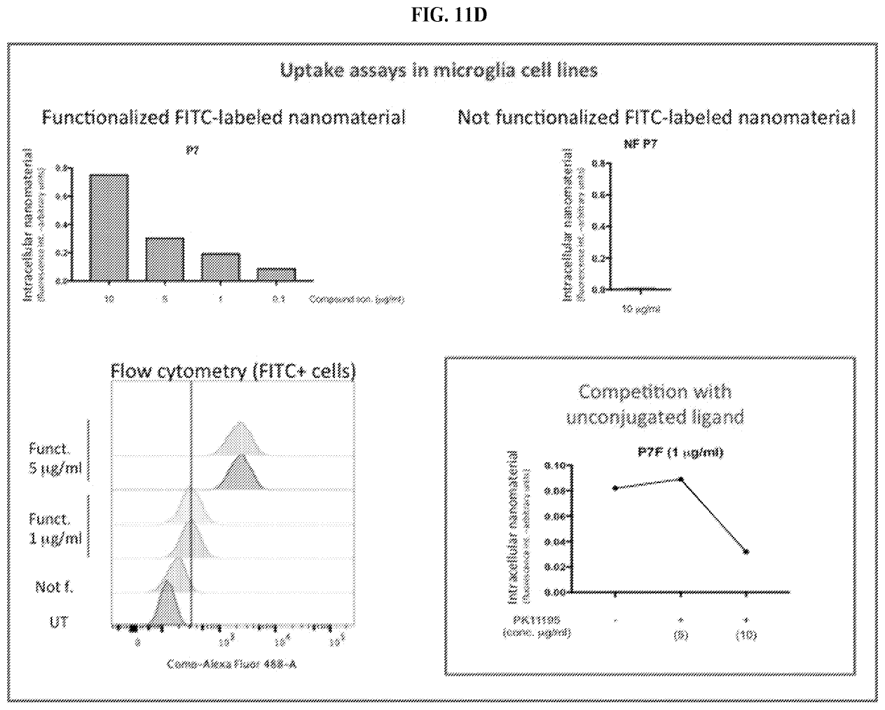

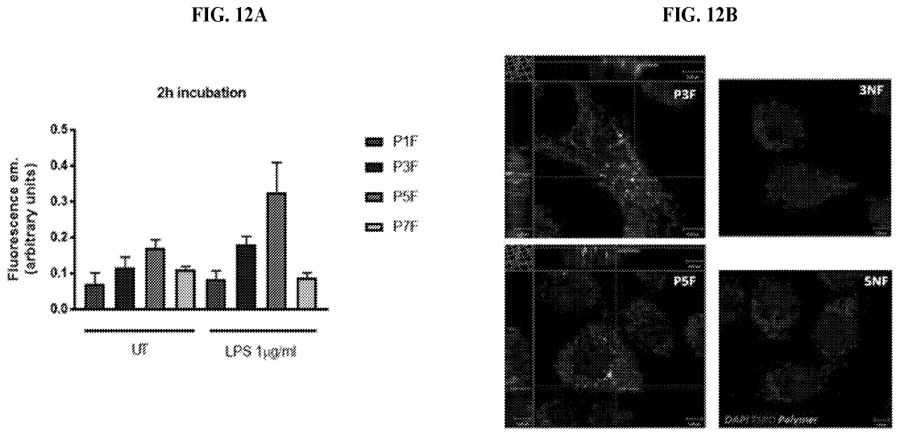

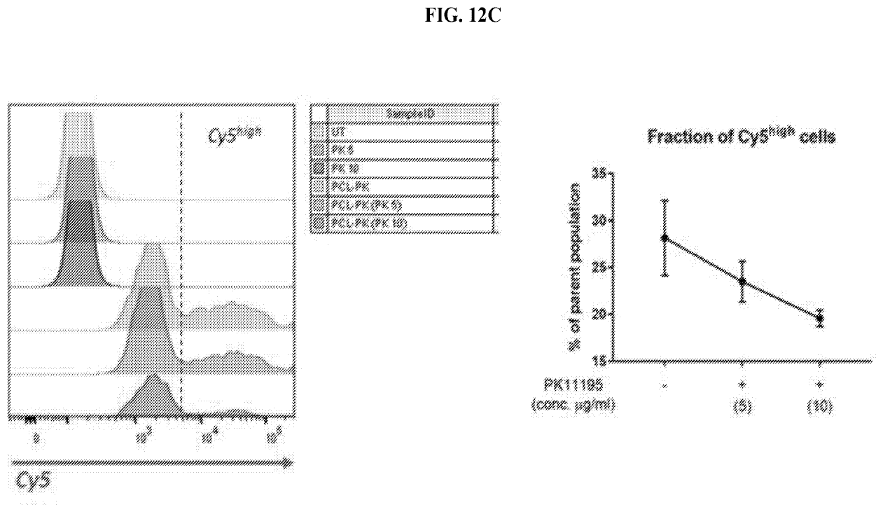

Compounds P1F, P3F, P5F, and P7F were added at different concentrations (in the range of 0.1-10 μg/ml) to the cell culture medium for 24 hours. Cells were collected and analyzed by spectrofluorimetry and flow cytometry to verify the uptake of the fluorescent labeled nanoparticles. Specificity of uptake was verified by: i) comparison with cells incubated with the respective fluorescent labeled non-functionalized polymers (namely P1-NF, P2-NF, P3-NF, and P4-NF, tested at 10 μg/ml); and ii) establishing a competitive pharmacological assay by co-incubating the functionalized polymers (tested at 1 μg/ml) with high concentrations (5 or 10 μg/ml) of the free unconjugated TSPO ligand PK11195 for 24 hours. As shown in FIGS. 11A-11D and summarized in Table 3, all of the four functionalized polymers displayed a good pharmacological profile. In terms of efficiency of the uptake, the level of uptake increased proportionally with the polymer concentration. Moreover, the uptake of the functionalized polymers was selective, as co-incubation of the conjugated polymers with increasing concentrations of the free ligand was able to disrupt the uptake, with P1F, P5F and P7F displaying the best profiles. Further testing in BV2 cells stimulated by lipopolysaccharide (LPS), to upregulate TSPO expression, confirmed that polymers P3F and P5F displayed the most promising uptake profile (FIG. 12A and Table 3). Without being bound by theory, their uptake appeared to be modulated by the level of engagement of the TSPO receptor. As shown in FIG. 12B, laser scanning confocal microscopy highlighted the presence of the FITC-labeled functionalized polymers (i.e., P3F or P5F) within the cytoplasm of BV2 cells and not the non-functionalized ones (P3-NF and P5-NF). The FITC⁺ signal was punctate and only partially co-localized with TSPO staining. Without being bound by theory, this indicates the accumulation of polymers in vesicle-like structures in close proximity to TSPO⁺ compartment. As shown in FIG. 12C, cells treated with different concentrations of PCL-PK11195 were distinct from cells treated with drug alone or untreated, and represented decreasing percentage of a parent population of cells with increasing PCL-PK11195 dosage.

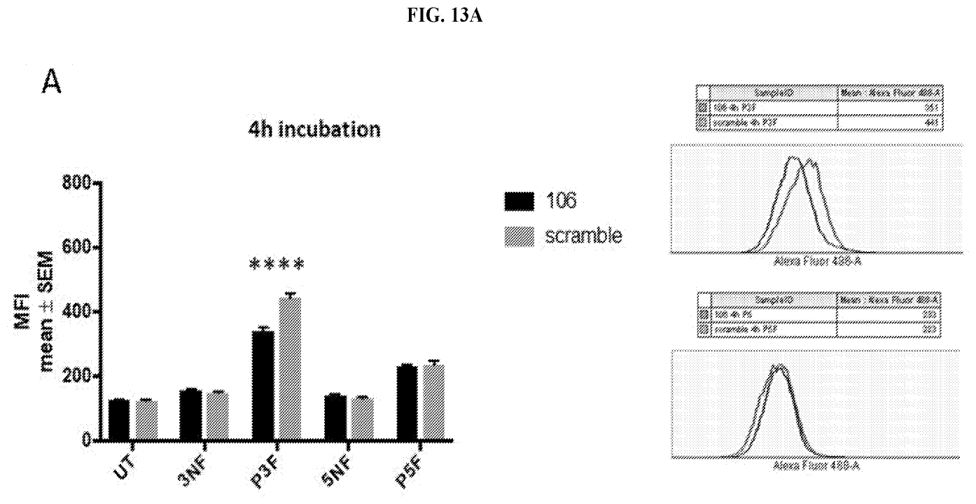

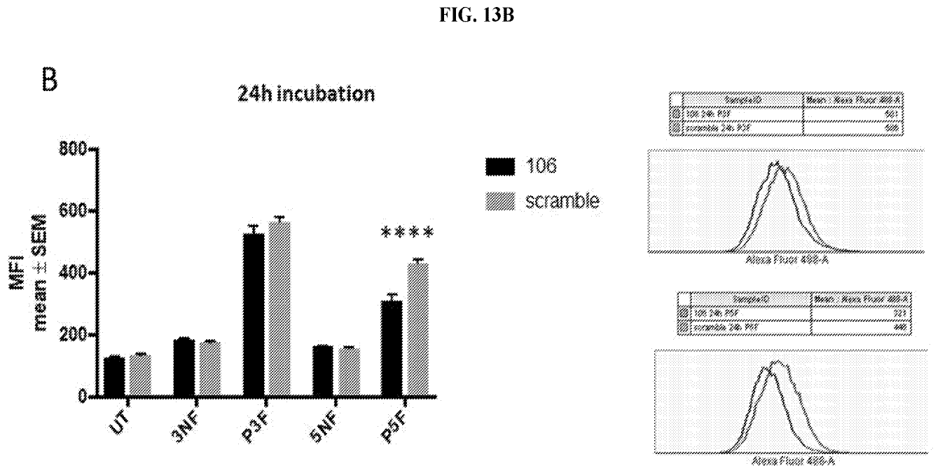

To further validate P3F and P5F in terms of selectivity for TSPO, their uptake was tested in the BV2 #106 cell line, which expresses only 20% of normal levels of TSPO due to infection with a lentiviral vector expressing a shRNA targeted to murine TSPO. BV2 #scramble (derived from infection with a lentiviral vector expressing a scrambled shRNA) was used as control. Briefly, 30,000 cells/well were plated in 48 wells plates one day before the experiment. Then, conjugated polymers P3F and P5F (1 μg/ml) were added to the cell culture medium. At 4 hours or 24 hours after treatment, the medium was aspirated, and cells were washed extensively with phosphate buffered saline (PBS) and collected by trypsinization for flow cytometric analysis of polymer uptake (detected by gating for FITC⁺ cells), as shown in FIGS. 13A-13B. A significant reduction in the uptake of P3F (about 20%) and P5F (about 30%) was observed in the BV2 #106 cell line. No uptake was confirmed for the corresponding non-functionalized polymers (i.e., 3NF and 5NF) in both cell lines. Without being bound by theory, the incomplete abrogation of P3F and P5F uptake in the #106 cell line was expected, as the residual levels of TSPO expressed in #106 cells can still account for uptake of the polymers. Interestingly, for P3F the uptake was inhibited mainly at 4 hours of incubation; in contrast, the uptake of P5 F was significantly inhibited only after 24 hours of incubation.

Without being bound by theory, differences in the affinity of P3F and P5F for TSPO receptor and/or in the dynamics of intracellular uptake may explain the discrepancies observed between the two polymers at different incubation time on the cells. Thus, a strategy was designed to assess the affinity of the polymers for TSPO.

### Example 8. Assessment of Binding of PK11195-Conjugated Polymers to TSPO Receptor by Microscale Thermophoresis (MST)

Microscale thermophoresis (MST) is a technique that detects changes in the hydration shell of molecules in solutions, by measuring the movement of biomolecules along microscale temperature gradients created by very low-power infrared-lasers within thin glass capillaries filled with analyte. The solvation entropy and the hydration shell of the molecules is the driving force of this phenomenon. Any changes of the hydration shell, due to modification of the structure of the biomolecules and/or interaction with a binding partner, affects the thermophoretic movement and is used to determine binding affinities. The advantage of this technique is that the measurement can be performed in close-to-native, immobilization-free conditions. Without being bound by theory, this is very important when dealing with transmembrane receptors (such as TSPO) or bulky molecules (such as polymeric nanoparticles) whose functionality may be affected by immobilization on a flat surface (a step required by other techniques such as surface plasmon resonance).

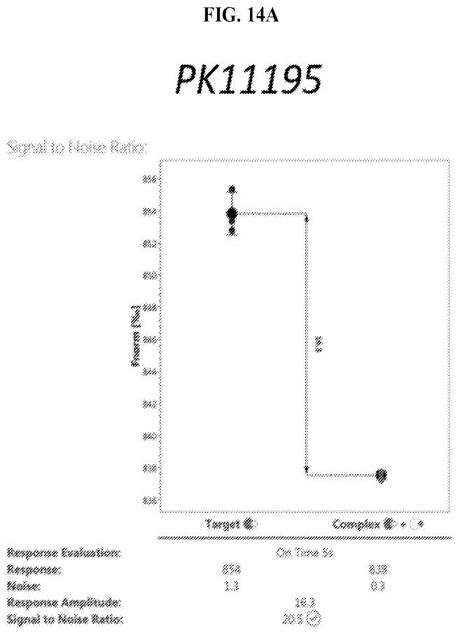

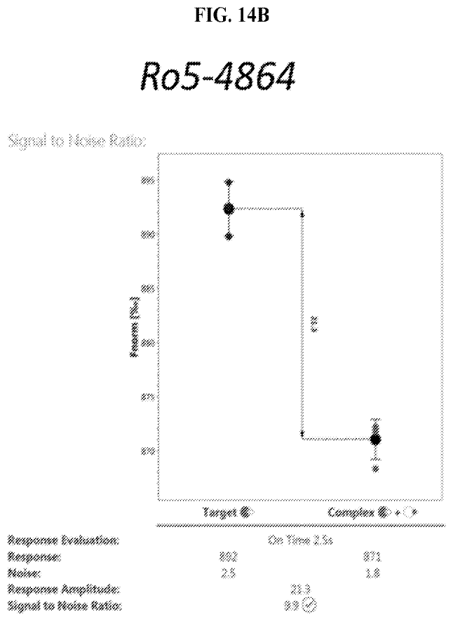

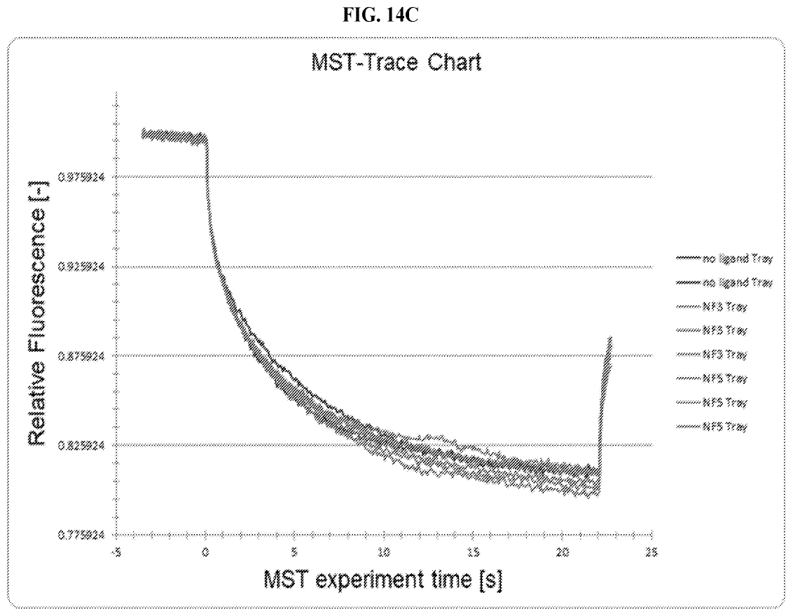

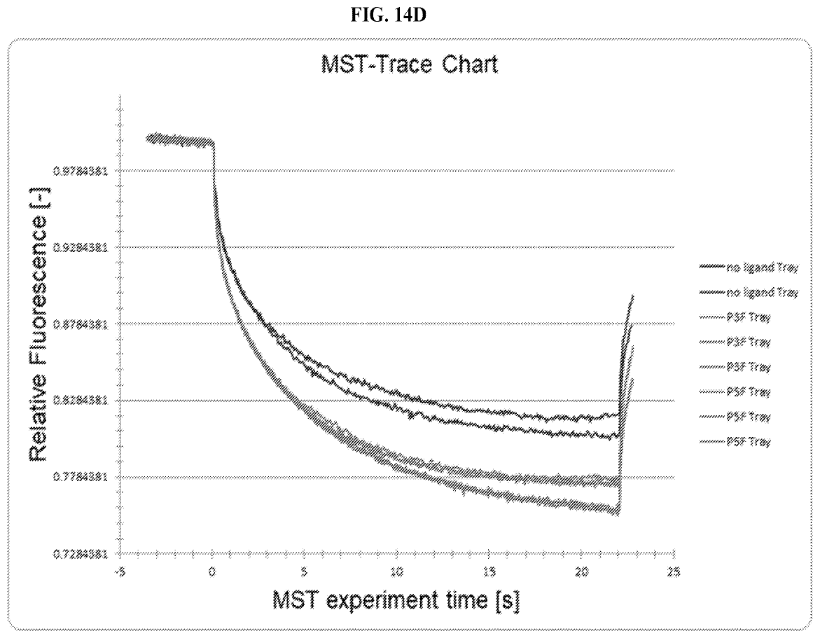

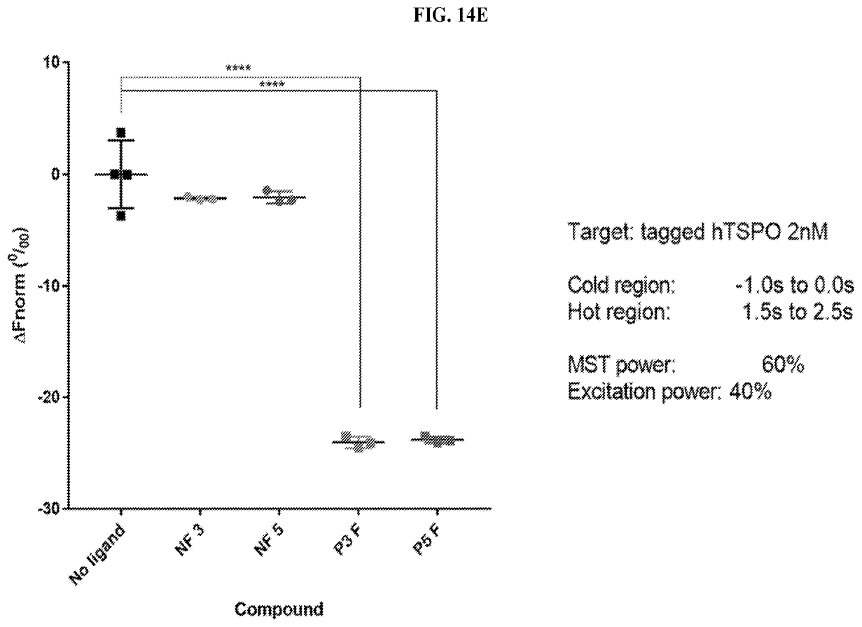

The tests were performed by using isolated human recombinant TSPO (purchased from LSBio). The lyophilized protein was resuspended in Tris buffer and stored at −80° C. as per manufacturer instructions. Labelling with a deep-red fluorophore was performed by using the Monolith labeling kit-RED from Nanothemper. Briefly, 200 nM of hTSPO was resuspended in Tris/Sarkosyl 0.1% buffer and then mixed with a solution containing anti-His red-fluorophore at 100 nM concentration. After incubation for 30 minutes at RT, the mixture was centrifuged at 15000 g for 10 minutes at 4° C., to pellet any aggregate or precipitate. For the assay, 5 nM red-tagged hTSPO was resuspended in TBS/DPC (12-dodecyl-phosphocoline) buffer and incubated with the free ligand PK11195 (tested at 250 μM) or PK11195-conjugated polymers P3 F or P5 F (tested at 500 μM). MST was then performed to assess binding. Non-functionalized polymers NFP3 and NFPS were also tested to validate the specificity of interaction with TSPO. Binding of Ro5-4864 (500 μM), another TSPO selective ligand, was used as a quality check to demonstrate the capability of the MST assay to assess binding for TSPO. As shown in FIGS. 14A-14B, a significant change in the MST profile of tagged hTSPO was detected in the presence of PK11195 or Ro5-4864. Without being bound by theory, this confirms the ability to exploit this technique to monitor binding of specific ligands to the isolated receptor. Interestingly, the MST assay performed on hTSPO in the presence of the PK11195 functionalized polymers P3F and P5F (FIG. 14D) or the non-functionalized counterparts (FIG. 14C) highlighted binding of the functionalized polymers to the receptor, as evidenced by the significant change in the MST profile (FIG. 14E). This confirms that the covalent conjugation of polymers with a TSPO-selective ligand did not affect binding with the receptor.

### Example 9. Synthesis and Characterization of MPC-PBR28 Conjugates

25MPC-4MePEG-N3 and 25MPC-8MePegN3 (FIG. 2) were polymerized through RAFT polymerization with the HemaCl5 and HemaRhodamine. The compounds were dissolved in Methanol and heated to 650 for 24 hours under stirring after degassing. The block copolymer was then precipitated in diethyl ether. The Copper(I)-catalyzed Azide-Alkyne Cycloaddition (CuAAC) reaction was used to couple the copolymer with a modified PBR-28 alkyne precursor (FIG. 9B) to realize functionalized copolymers. Fourier-transform infrared spectroscopy (FTIR) was used to monitor the disappearance of the peak referred to N3 group from the sample as confirmation of the conjugation of the PBR-precursor to the polymer. The block copolymer so created was then nanoprecipitated to produce nanoparticles. Different batches were produced, with different PBR-ligand to NP molar ratios, as outlined in Table 4. In detail: i) NP-MPC batches with 6 units of PegN3 per MPC polymer (batches number #2_x, where x is a number between 1 and 2) will display more sites available for functionalization with PBR-click, as compared to NP-MPC batches carrying only 4 units of PegN3 per MPC polymer (batches number #1_x, where x is a number comprised between 1 and 2); ii) NP-MPC batches with 30 units of HemaCLS per MPC polymer (batches number #x_1, where x is a number comprised between 1 and 2) will have a core that is less lipophilic than the NP-MPC batches with 60 units of HemaCL5 per MPC polymer (batches number #x_2, where x is a number comprised between 1 and 2).

TABLE 4
Chemical characteristics of produced MPC-PBR batches.

| | Diam- | MPC | PegN3 | HemaCL5 | Func- |
|---|---|---|---|---|---|
| | eter | (units/ | (units/ | (units/ | tional- |
| Batch ID | (nm) | polymer) | polymer) | polymer) | ization |
| MPC-PBR 2_1 | 100 | 25 | 6 | 30 | PBR- |
| | | | | | click |
| MPC-PBR 2_2 | 100 | 25 | 6 | 60 | PBR- |
| | | | | | click |
| MPC-PBR 1_1 | 100 | 25 | 4 | 30 | PBR- |
| | | | | | click |
| MPC-PBR 1_2 | 100 | 25 | 4 | 60 | PBR- |
| | | | | | click |
| MPC-NF 2_1 | 100 | 25 | 6 | 30 | none |
| MPC-NF 2_2 | 100 | 25 | 6 | 60 | none |
| MPC-NF 1_1 | 100 | 25 | 4 | 30 | none |
| MPC-NF 1_2 | 100 | 25 | 4 | 60 | none |

Ligand-dependent uptake was tested on BV2 microglia cell line to identify the formulation allowing best selectivity of uptake. NP-MPC, either functionalized with PBR-28 click precursor (MPC-PBR) or non-functionalized (MPC-NF), were added at different concentrations (in the range of 0.1-5.0 μg/ml) to the cell culture medium for 4 hours or 24 hours.

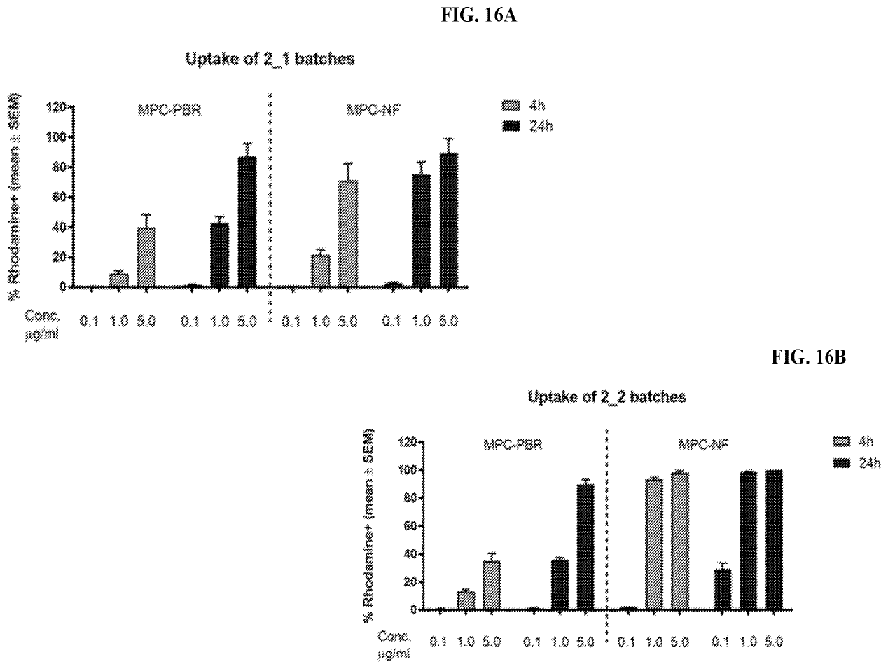

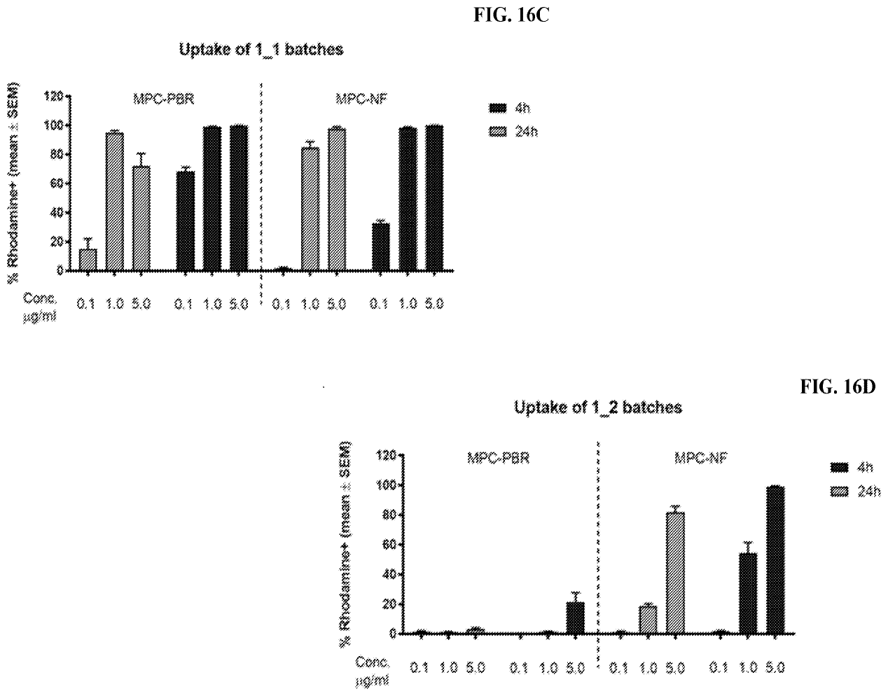

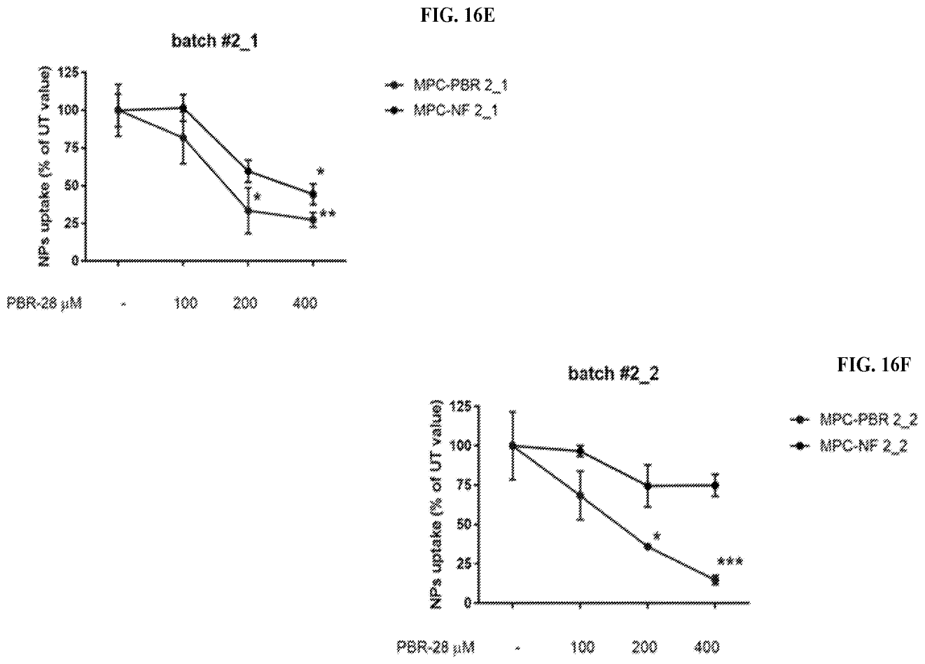

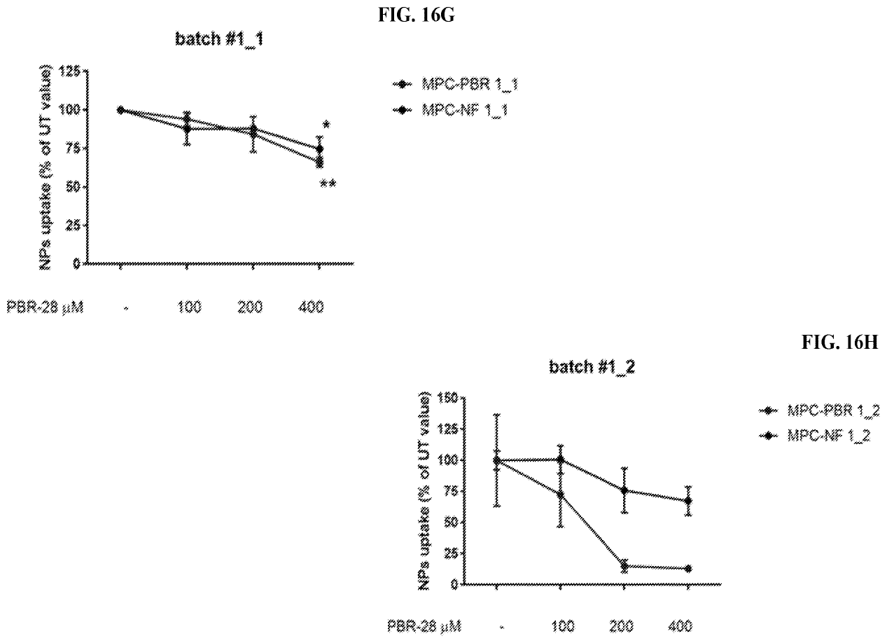

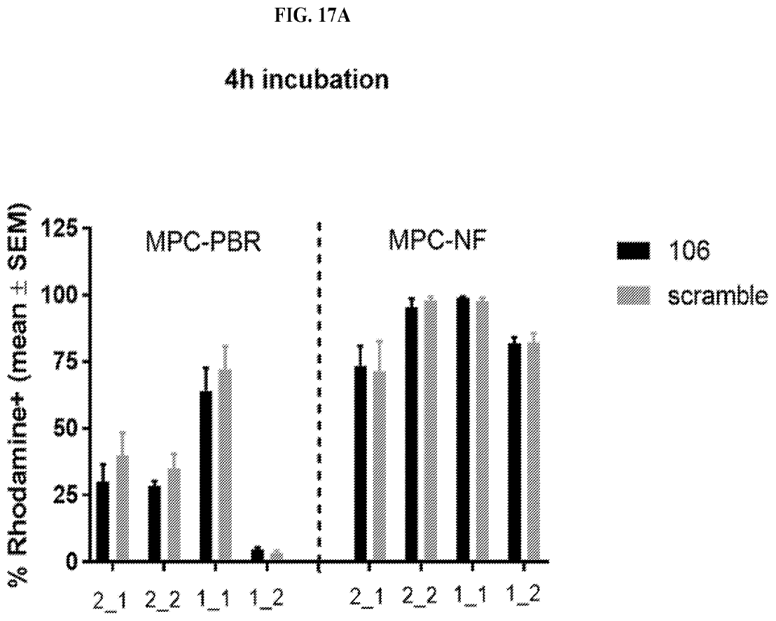

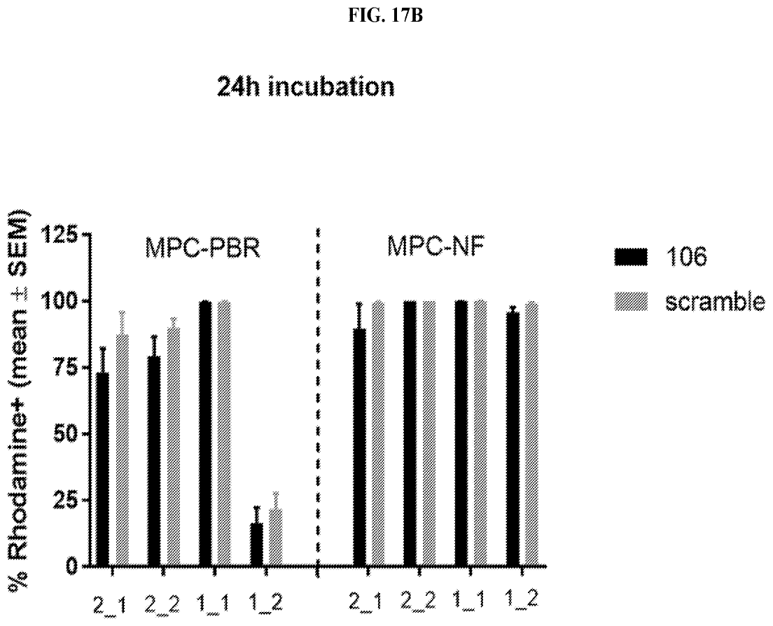

Cells were collected and analyzed by flow cytometry to verify the uptake of the fluorescent labeled NPs. In line with what was observed for other non-functionalized NP-MPC (see FIG. 3A), a dose- and time-dependent increase of the uptake of both functionalized and non-functionalized NPs was observed (FIGS. 16A-16D). To verify whether the uptake of PBR-functionalized NPs is mediated by TSPO-receptor, the uptake of functionalized (MPC-PBR) versus non-functionalized (MPC-NF) NPs was compared in the context of a competitive pharmacological assay by co-incubating the functionalized NPs (tested at 5 μg/ml) with increasing concentrations (100, 200, and 400 M) of free unconjugated TSPO ligand PBR28 for 4 hours. As shown in FIGS. 16E-16H, the uptake of all four MPC-PBR formulations was disrupted by co-incubation with increasing concentrations of the free ligand, with #2_2 displaying the best profile in terms of dose-response and selectivity, in comparison to the corresponding non-functionalized NP formulations. To further validate the selectivity for TSPO, uptake was tested in the BV2 #106 cell line (expressing only 20% of normal levels of TSPO), as previously described. Functionalized and non-functionalized NPs (5 μg/ml) were added to the cell culture medium and the uptake was analyzed by flow cytometry at 4 hours or 24 hours after treatment. As shown in FIGS. 17A-17B, a tendency to a reduction of the uptake was confirmed for NP-MPC functionalized batches #2_1 and #2_2 both at 4 hours and 24 hours of incubation in the BV2 #106 cell line. In contrast, no change in the uptake of any of the non-functionalized batches was detected. This supports an important role for the surface functionalization with PBR-click to allow the TSPO-dependent uptake of NPs batches #2_1 and #2_2.

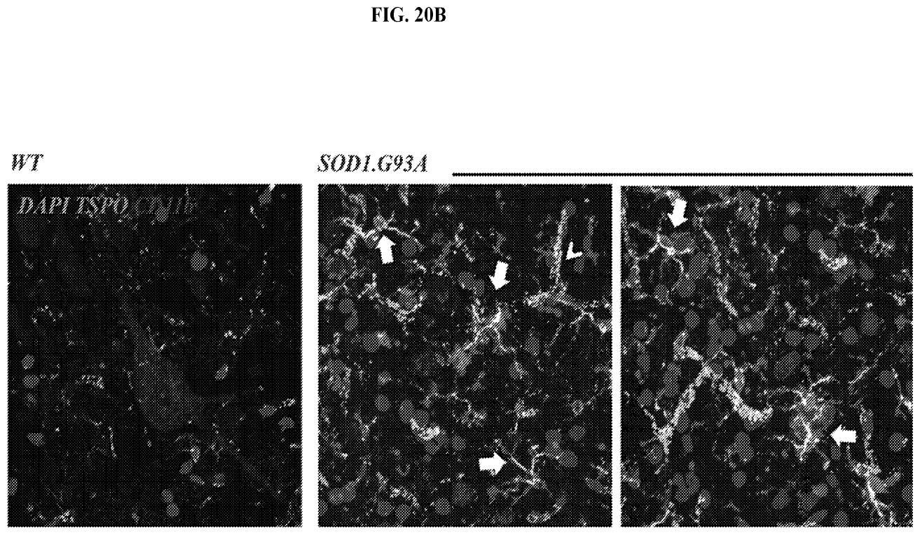

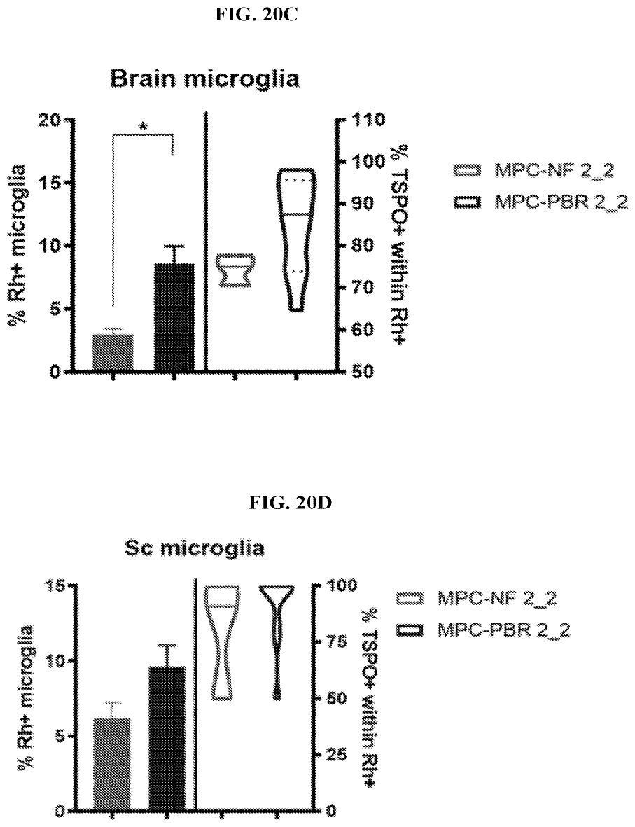

The functionalized MPC-PBR 2_2 and the corresponding not-functionalized MPC-NF 2_2 NPs were administered to symptomatic transgenic SOD1.G93A mice (a widely used mouse model of ALS) at the symptomatic stage of the disease (when TSPO is significantly upregulated in microglia cells throughout the brain and spinal cord, FIGS. 20A, 20B). As shown in FIGS. 20C and 20D, a higher uptake of the functionalized MPC-PBR 2_2 NPs is reported in brain and spinal cord. The majority of cells internalizing the NPs are TSPO⁺, consistent with the increased targeting of TSPO⁺ cells by functionalized MPC-NPs.

Overall, these experiments support the NP platform hereby described, which was designed to allow functionalization with receptor-targeted ligands, as a tool to achieve selective cellular uptake and multiple drug release.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents, publications, and accession numbers mentioned in this specification are herein incorporated by reference to the same extent as if each independent patent, publication, and accession number was specifically and individually indicated to be incorporated by reference.

1. (canceled)

2. A method of making a functionalized polymer comprising contacting a first monomer: 2-Methacryloyloxyethyl phosphorylcholine (MPC), and a second monomer: methacrylate/amide PEG comprising a functionalized amine or azide, in the presence of a radical and a thiocarbonylthio compound.

3. The method of claim 2, wherein the functionalized polymer comprises Formula I

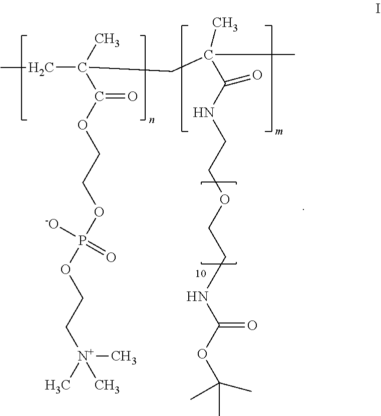

4-6. (canceled)

7. The method of claim 2, wherein the functionalized polymer comprises Formula II:

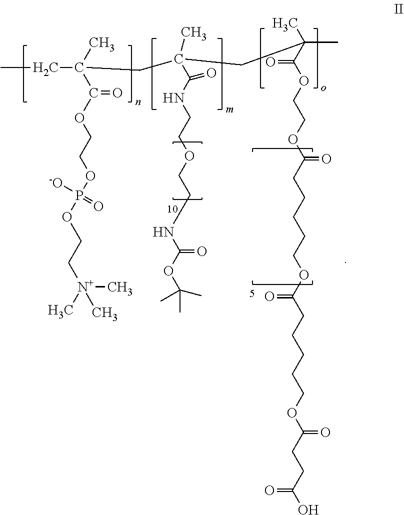

8-12. (canceled)

13. A functionalized polymer comprising a TSPO ligand or precursor or analog thereof covalently linked to a branched PEG polymer.

14-20. (canceled)
